SEQUENCE LISTING (39 sequences)

SEQ ID NO: 1 - DNA, 1265 bp, Sphaerodes mycoparasitica
atagggagaagaagcactgcgattgccctagtaacggcgagtgaagcggcagcagcccagatttggaatctggtccttttggggcccgagttgtaatctgcagaggaagcgtctggtgcggtgccggcctagttccctggaacgggacgccgtagagggtgacagccccgtacggtcggccaccaaacctgtgtgtcgctccttcgaagagtcgcgtagtttgggaatgctgcgtaaagtgggaggtatgctcctcctaaggctaaataccggccagagaccgatagcgcacaagtagagtgatcgaaagatgaaaagcaccttgaaaatggggttaaaaagtacgtgaaattgccaaaggggaagcgctcgtggccagactcgtgccttatggatcatccggctatttcgccggtgcactccattaggctcgggccagcgtcggtcggcgccggtactaaaagacagcgcgaacgtggctctcttcggggagtgttatagcgcgctgtgtaatgtgctggcgccgtccgaggaccgcgcatttatgcaaggacgctggcgtaatggccactagcgacccgtcttgaaacacggaccaaggagtcgcccagagacgcgagtgtgcgggtgacaaacccctgcgcgaagtgaaagcgaacgctggtgggaaccctcacgggtgcaccaccgaccgatcctgatgtcttcggatggatttgagtatgagcgtttctggtcggaccgaaagagggtgaactatgcttgggtagggtgaagccagaggaaactctggtggaggccccgtttgggttctgacgtgcaaatcgatccataaacctgggcatagcggcgaaagactaatcgaaccttctagtagctggttcgcattctctctctcgcacgagagagagaaaacctctgtgatatcacgattatcagtgaaaaccacaccgagacccaacggagttcttctggatttcctcatgcttcaattaccacgcctagtggacctacctggagcgctacaataaagtcatacgaaaatctcgaagatcggggtgacggtgagggatcctaaggttctctcgttgagtgcgttggacgggcatggccgtcagcgatctggggcgaccgttgccggatcataagggctttagtgcttaggctattggtattgaggggtctgaagacggtaatctgaaaccaaaggctttattctaaacccgcgcagcatgggcgtagtaggaagagacagcgaagtctag

SEQ ID NO: 2 - DNA, 1233 bp, Sphaerodes mycoparasitica
agtgcggcatgttgtagcctaagcaattatacagcgaaactgcgaatggctcattatataagttatcgtttatttgatagtgccttactacttggataaccgtggtaattctagagctaatacatgctgaaagccccgacttacggaggggcgtatttattagattaaaaaccaatgcccttcggggctctttggtgattcatgataacttctcgaatcgcacggccttgcgccggcgatggttcattcaaatttcttccctatcaactttcgatgtttgggtagtggccaaacatggtggcaacgggtaacggagggttagggctcgaccccggagaaggagcctgagaaacggctcctacatccaaggaaggcagcaggcgcgcaaattacccaatctcaactcgaggaggtagtgacaataaataccgatgcagggctctttagggtcttgcaattggaatgagtacaatttaaatcccttaacgaggaacaattggagggcaagtctggtgccagcagccgcggtaactccagctccaatagcgtatattaaagttgttgtggttaaaaagctcgtagttgaaccttgggcctggccggctggtccgcctaacagcgtgcactggtgcggccgggtcttcccaccgcggagccgcatgtccttcactgggcgtgtcggggaagcggtacttttactgtgaaaaaattagagtgctctaagcaggcctatgctcgaatacattagcatggaataatagaataggacagtcgttctattttgttggtttctaggacgtctgtaatgattaacagaaacaatcgggggcgtcagtattgcatcgtcagaggtgaaattcttagatcgatgcaagactaactactgcgaaagcattcgccaagggtgttttcattaatcaggaacgaaagttaggggatcgaagacgatcagataccgtcgtagtcttaaccataaactatgccgactagggatcgggcggtgtaattttgacccgctcggcacttacgagaaatcttaagtgcttgggctccaggggagtatggtcgcaaggctgaaacttaaagaaatgacgaagggcaccaccaagggtgaacctgcggcctagttgactcacacgggaaactcacgaggtaggaatatgtagatgacggatggagccttcagaatacatatgggcatgcgctcttaataccggtattgaaaatgggcag

SEQ ID NO: 3 - DNA, 560 bp, Sphaerodes mycoparasitica
ttcgtttcttatcgcattggtgaccagcggagggtcattacgaatcggaccatttatgtcatggctctgccaaccctgtgaactttatacttgtacgttgcctcggcggaacctgccttttcggcaggccgccggccggcatatacgcaaacgctctgaaaaagctccgcgctctatctgaataataaaactttaacgagtaaaaacttttggcaacggatctcttggctctggcatcgatgaaaaacgcagcgaaatgcgatacgtaatgtgaattgcagaattcagtgaaccatcgaatctttgaacgcaccttgcggccgccggtaatccggcggccatgcccgtccgagcgtcgtttccaccctcgggagttctcctcctaagaaaatttctcccggccttgggccagcgcgttgcgcggctgcccgaccaacggcggcaggaccggcgatgtcctctgtgccctgcatttatataaaactcgcattggtccccggtaaggcttgccttgcaaccaacttctttaggtcgacctcagatcggatagggatacccgc

SEQ ID NO: 4 - DNA, 23 bp, Sphaerodes mycoparasitica
tcatggctctgccaaccctgtaa

SEQ ID NO: 5 - DNA, 23 bp, Sphaerodes mycoparasitica
aatgcagggcacagaggacatcg

SEQ ID NO: 6 - DNA, 21 bp, Fusarium graminearum
taaacgcccgcgaagttcaca

SEQ ID NO: 7 - DNA, 20 bp, Fusarium graminearum
tggtgatggttcgcttcgag

SEQ ID NO: 8 - DNA, 20 bp, Fusarium graminearum
agcgactacaggcttccctc

SEQ ID NO: 9 - DNA, 22 bp, Fusarium graminearum
aaaccatccagttctccatctg

SEQ ID NO: 10 - DNA, 24 bp, Fusarium graminearum
tctctaccaacggtggattcaacc

SEQ ID NO: 11 - DNA, 22 bp, Fusarium graminearum
agcctttggtgccgacttcttg

SEQ ID NO: 12 - DNA, 22 bp, Fusarium graminearum
tctgaacaggcgatggtatgga

SEQ ID NO: 13 - DNA, 21 bp, Fusarium graminearum
ctgcggcgagtgagtttgaca

SEQ ID NO: 14 - DNA, 24 bp, Fusarium graminearum
gtgggcttcgctagaccgtgagtt

SEQ ID NO: 15 - DNA, 22 bp, Fusarium graminearum
atgccctgatgaagagtttgat

SEQ ID NO: 16 - DNA, 24 bp, Fusarium graminearum
cccccaactcgacgtcaaatctat

SEQ ID NO: 17 - DNA, 23 bp, Fusarium graminearum
ttcttcccgccgacttcaaaaca

SEQ ID NO: 18 - DNA, 21 bp, Fusarium graminearum
ggtctcgacagcaatggtgtt

SEQ ID NO: 19 - DNA, 21 bp, Fusarium graminearum
gcttgtgtttttcgtggcagt

SEQ ID NO: 20 - DNA, 20 bp, Fusarium graminearum
tcgagtttcgtgttgcgtgt

SEQ ID NO: 21 - DNA, 20 bp, Fusarium graminearum
aggtagttcgccatacccgt

SEQ ID NO: 22 - DNA, 20 bp, Fusarium graminearum
aattgtgcccgaggcagtac

SEQ ID NO: 23 - DNA, 21 bp, Fusarium graminearum
cattggttccgcccgcaatag

SEQ ID NO: 24 - DNA, 21 bp, Fusarium graminearum
tgacaacttcgctggtttgga

SEQ ID NO: 25 - DNA, 21 bp, Fusarium graminearum
catagcttggccagtgccatc

SEQ ID NO: 26 - DNA, 20 bp, Fusarium graminearum
gaagtcattcggtgttgagc

SEQ ID NO: 27 - DNA, 21 bp, Fusarium graminearum
gctctggattgggtatcgcac

SEQ ID NO: 28 - DNA, 21 bp, Fusarium graminearum
actcgagcatccgtcgcaatg

SEQ ID NO: 29 - DNA, 20 bp, Fusarium graminearum
agcaacatctccgtctggag

SEQ ID NO: 30 - DNA, 20 bp, Fusarium graminearum
gttgaactgtccatggctga

SEQ ID NO: 31 - DNA, 20 bp, Fusarium graminearum
gaatgaaggcaatctgctgc

SEQ ID NO: 32 - DNA, 24 bp, Fusarium graminearum
acctcactggaatcagagcgcagc

SEQ ID NO: 33 - DNA, 20 bp, Artificial Sequence
  AuroRTR primer
atgacracttcccgtggrcc

SEQ ID NO: 34 - DNA, 21 bp, Fusarium graminearum
ggtctcgacagcaatggtgtt

SEQ ID NO: 35 - DNA, 21 bp, Fusarium graminearum
gcttgtgtttttcgtggcagt

SEQ ID NO: 36 - protein, 15 aa, Sphaerodes mycoparasitica
YLPGGGGGRDEPPPR

SEQ ID NO: 37 - protein, 9 aa, Sphaerodes mycoparasitica
LHVQFMSSK

SEQ ID NO: 38 - protein, 29 aa, Sphaerodes mycoparasitica
EIAVTELDIAGASSTDYVEVVEACLNQPK

SEQ ID NO: 39 - protein, 12 aa, Sphaerodes mycoparasitica
TTVSYDTGYDDK

(19) United States
(12) Patent Application Publication
(10) Pub. No.: US 2012/0156173 A1
(43) Pub. Date: Jun. 21, 2012

(54) FUSARIUM AND OTHER PATHOGENIC FUNGI AND MYCOTOXIN BIOCONTROL

(76) Inventor: Vladimir Vujanovic, Saskatoon (CA)

(21) Appl. No.: 13/405,771

(22) Filed: Feb. 27, 2012

Related U.S. Application Data
(63) Continuation-in-part of application No. PCT/CA2010/001253, filed on Aug. 30, 2010.
(60) Provisional application No. 61/237,906, filed on Aug. 28, 2009.

(30) Foreign Application Priority Data
Feb. 25, 2011 (CA) PCT/CA2011/000208

(51) Int. Cl. A61K 36/062; A01N 63/04; C07K 7/08; A61P 31/10; C07K 14/37; C12N 15/31; C12Q 1/68; A01P 3/00; C12N 1/14; C07K 7/06

(52) U.S. Cl. 424/93.5; 435/254.1; 530/326; 530/328; 530/324; 530/327; 530/371; 536/23.74; 435/6.12

(57) ABSTRACT

The present disclosure relates to a novel ascomyceteous fungus, Sphaerodes mycoparasitica, for controlling plant fungal pathogens, disease symptoms, and mycotoxins in planta and ex planta. Uses, methods, compositions, sequences, and products are also disclosed herein.

### RELATED APPLICATIONS

This application is a continuation-in-part of PCT/CA2010/001253 filed on Aug. 30, 2010, which claims the benefit of priority from U.S. provisional application No. 61/237,906 filed Aug. 28, 2009, the contents of both of which are incorporated herein by reference in their entirety. This application also claims the benefit of priority from PCT/CA2011/000208 filed on Feb. 25, 2011, the contents of which are incorporated herein by reference in its entirety.

### INCORPORATION OF SEQUENCE LISTING

A computer readable form of the Sequence Listing “13764-165_SequenceListing.txt” (16,384 bytes), submitted via EFS-WEB and created on Feb. 27, 2012, is herein incorporated by reference.

### FIELD

The present disclosure relates to novel biocontrol agents, related compositions with fungicidal and fungistatic, antifungal and antimycotoxin effect and uses thereof. In particular, the present agent has a fungicidal and fungistatic effect on pathogenic fungi, such as members of the genera Fusarium, Sclerotinia, Rhizoctonia, Pythium, and the like. The present invention further relates to the isolated fungal inoculant, genes, proteins and/or organisms as well as uses, methods, compositions, involving the same.

### BACKGROUND

Fusarium is a filamentous fungus widely distributed on plants and in the soil. Certain Fusarium species are plant pathogens. For example, Fusarium oxysporum causes Fusarium wilt disease in more than a hundred species of plants. It does so by colonizing the plant xylem which can result in blockage and breakdown. When this occurs, symptoms such as leaf wilting and yellowing appear in the plant eventually leading to the plant's death. This condition was the primary cause of the decline and disappearance of the Gros Michel banana cultivar from markets around the world. Recently a new strain has begun attacking plants of the dominant Cavendish banana cultivar leading to fears that, in the absence of a solution, this cultivar will too disappear from world markets.

Fusarium root rot is a major cause of seedling mortality in forest nurseries and also causes reduced survival after outplanting during the first growing season. The disease is caused by several Fusarium species and is common in many parts of the world. The disease is a particular problem in Western Canada and the United States and also in the North Central and Southern States. In addition, Pine pitch canker—caused by the fungus Fusarium circinatum—is a serious disease of pine trees and a threat to the forest industry, particularly in the US and New Zealand. Radiata or Monterey pine is highly susceptible to the disease with mortality rates in mature trees reaching 80% in some areas of California.

Fusarium Head Blight (FHB), also known as ‘ear blight’, ‘tombstone’, or ‘scab’, is a disease of wheat, barley, oats and other small cereal grains caused by Fusarium. Corn or maize can be affected by a similar condition known as ‘ear rot’. The aforementioned condition can reduce the yield and grade of the crop, and can potentially contaminate the grain with mycotoxins. It is estimated that FHB costs the cereal industry almost $5 billion annually. In recent years, FHB has proved a significant and growing problem for the commercially important wheat and barley crops in Western Canada. In the past Fusarium graminearum and Fusarium avenaceum have been identified as the two species primarily associated with FHB, whereas Fusarium oxysporum have caused important asparagus decline and tomato wilt outbreaks in Canada wherein yields in some affected fields have been reduced by 30% or more. FHB and ear rot harvested grain is often contaminated with mycotoxins such as deoxinivalenol (DON) trichothecenes associated with feed refusal, general digestive disorders, diarrhea, and hemorrhages. The emergence of the toxigenic 3-acetyldeoxynivalenol (3-ADON) Fusarium graminearum population in North America is a fairly recent phenomenon. It is replacing the 15-ADON chemotype. Moreover, the mycotoxin profiles of F. culmorum—similar to F. graminearum under both laboratory and field conditions—represents an additional threat for corn, maize and wheat production worldwide including but not limited to South American, African, Australian, European, Asian regions of the Russian Federation, and China. Currently, there are no effective control measures for FHB and associated mycotoxins. Additionally, there are no resistant varieties of corn, wheat, barley, oats, or other small grain cereals. Fungicides can be effective but only temporarily suppress the disease.

In addition to being a common plant pathogen, Fusarium spp. can opportunistically infect animals and can be the causative agents of superficial and/or systemic infections in humans. Fusaria are one of the most drug-resistant fungi making fusarial infections difficult to treat. Invasive infections can prove fatal.

Sclerotinia stem rot is caused by the pathogen Sclerotinia sclerotiorum. S. sclerotiorum has a wide host range and is known to infect numerous species of plants (canola, sunflower, soybean, flax, etc.). Disease outbreaks can be particularly severe under conditions of zero crop rotation or if rotations include several susceptible plant species. Total loss of the potential yield can occur when infections occur early in the flowering period. Another wide-spread fungal plant pathogen is Rhizoctonia spp. which causes “damping off” or “wire-stem” disease symptoms in crop plants. Infection can occur at any time during the growing season, but the seedling stage is most susceptible. Incidence and severity of disease at all growth stages are influenced by weather, soil conditions and inoculum levels. Seedling infection is favoured by cool weather. As well, incidences of wirestem disease are most severe in spring and fall when soils are wet and cool. Root rots generally occur during periods of warm, wet weather and may affect the plant at any stage of development. Seedlings affected by damping-off fail to emerge or if they do, they quickly decline, topple over and die. In older seedlings, there is purpling on the lower leaves and the lower stem becomes constricted and dark-brown near the soil surface. Other symptoms may include seed decay, rotting roots and cankers on lower petioles. Pythium ultimum is a ubiquitous soilborne pathogen widely distributed throughout the world, which causes damping-off and root rot on plants. P. ultimum has a wide range of hosts including many important agronomic crops and turf grasses. Similarly to Fusarium and Rhizoctonia species, Pythium spp. cause severe damping-off in seedlings of forest nurseries.

### SUMMARY

The present disclosure relates to a novel ascomyceteous fungus, Sphaerodes mycoparasitica, such as that identified as strain IDAC 301008-01 and alternatively as strain SMCD2220-01, that has a mycoparasitic and antimycotoxin effect. In addition, mycotoxic proteins have been isolated from S. mycoparasitica strains IDAC 301008-01, IDAC 301008-02 and IDAC 301008-03. IDAC 301008-01 is an isolated strain of S. mycoparasitica, IDAC 301008-02 is a co-culture of S. mycoparasitica and Fusarium graminearum, and IDAC 301008-03 is a co-culture of S. mycoparasitica with F. avenaceum. Both co-cultures are characterized by an elevated antimicrobial protein content. All three strains control fungal pathogens and/or disease symptoms caused by plant pathogenic fungi, such as Fusarium spp., Sclerotinia spp., Rhizoctonia spp., Pythium spp., and the like. These proteins also have anti-mycotoxin effects on toxins produced by pathogenic fungi exemplified by Fusarium spp., Sclerotinia spp., Rhizoctonia spp., Pythium spp., and the like, such as other pathogenic generalists, omnipresent pathogens on wide rage of host plants. Uses, methods, compositions, sequences, and products are also disclosed herein.

In one embodiment, the present disclosure provides an isolated culture of Sphaerodes mycoparasitica, wherein the species is characterized by a combination of:
(a) ascospore size, shape (fusiform and triangular) and wall ornamentation (reticulate and smooth);
(b) conidia produced from simple phialides on the surface of ascoma peridial wall on ascoma surrounding hyphae, and on irregularly branched conidiophores arising from hyphae; and
(c) forming hook-like structures parasitizing living hyphae of Fusarium.

In another embodiment, the isolated culture of Sphaerodes mycoparasitica comprises a gene encoding a large subunit of ribosomal RNA gene as shown in SEQ ID NO:1 or a variant thereof; a gene encoding a small subunit of ribosomal RNA as shown in SEQ ID NO:2 or a variant thereof; or a gene encoding an internal transcribed spacer ribosomal DNA as shown in SEQ ID NO:3 or a variant thereof.

In a particular embodiment, the isolated culture of Sphaerodes mycoparasitica is Sphaerodes mycoparasitica strain IDAC 301008-01. In another embodiment, the isolated culture of Sphaerodes mycoparasitica is from Sphaerodes mycoparasitica strain IDAC 301008-02. In yet another embodiment, the isolated culture of Sphaerodes mycoparasitica is from Sphaerodes mycoparasitica strain IDAC 301008-03.

Also provided herein is a method of controlling plant pathogenic fungi comprising administering the culture of Sphaerodes mycoparasitica disclosed herein to a subject in need thereof. The plant pathogenic fungus optionally is Fusarium spp., Sclerotinia spp., Rhizoctonia spp. or Pythium spp. In an embodiment, the subject is a plant. In another embodiment, the subject is an animal.

In another embodiment, the present disclosure provides a method of modulating synthesis of one or more of the following Fusarium mycotoxins: trichothecene mycotoxin deoxynivalenol (DON), 3-ADON, 15-ADON, zerelanone, and aurofusarin, comprising administering the culture of Sphaerodes mycoparasitica disclosed herein to a subject in need thereof.

In yet another embodiment, the present disclosure provides a method for detoxifying food, feed, or an environmental sample comprising one or more of a Fusarium trichothecene mycotoxin deoxynivalenol (DON), mycotoxin 3-ADON, mycotoxin 15-ADON, mycotoxin zerelanone, and mycotoxin aurofusarin comprising administering the culture of Sphaerodes mycoparasitica disclosed herein to said food, feed, or environmental sample.

Further provided herein is a composition comprising the culture of Sphaerodes mycoparasitica disclosed herein and a carrier and optionally further comprising an additional antifungal agent. The compositions may be seed treatment compositions, plant treatment compositions, or soil treatment compositions.

Further provided is a method of modulating synthesis of one or more of the following Fusarium mycotoxins: trichothecene mycotoxin deoxynivalenol (DON), 3-ADON, 15-ADON, zerelanone, and aurofusarin comprising administering the composition disclosed herein to a subject in need thereof.

Also provided herein is a method for controlling pathogenic fungi in plants, the method comprising treating a batch of seeds with the culture or compositions disclosed herein and then culturing the treated seeds into plants.

The present disclosure also provides an isolated protein comprising the amino acid sequence as shown in SEQ ID NO:36, SEQ ID NO:37, SEQ ID NO:38 or SEQ ID NO:39 or a variant thereof. In one embodiment, the variant has 80% identity to SEQ ID NO:36, SEQ ID NO:37, SEQ ID NO:38 or SEQ ID NO:39. In another embodiment, the isolated protein comprises SEQ ID NO:36, SEQ ID NO:37, SEQ ID NO:38 or SEQ ID NO:39.

In one embodiment, the protein is an exocellular protein recoverable from a culture of Sphaerodes mycoparasitica strain IDAC 301008-01, -02, or -03, wherein said exocellular protein comprises the amino acid sequence as shown in SEQ ID NO:39 and has a molecular weight of 13 kDa. In another embodiment, the protein is an exocellular protein recoverable from a culture of Sphaerodes mycoparasitica strain IDAC 301008-01, -02, or -03, wherein said exocellular protein comprises the amino acid sequence as shown in in SEQ ID NO:38 and has a molecular weight of 36 kDa. In yet another embodiment, the protein is an exocellular protein recoverable from a culture of Sphaerodes mycoparasitica strain IDAC 301008-01, -02, or -03, wherein said exocellular protein comprises the amino acid sequence as shown in in SEQ ID NO:37 and has a molecular weight of 50 kDa. In yet another embodiment, the protein is an exocellular protein recoverable from a culture of Sphaerodes mycoparasitica strain IDAC 301008-01, -02, or -03, wherein said exocellular protein comprises the amino acid sequence as shown in SEQ ID NO:36 and has a molecular weight of 79 kDa.

In another embodiment, the present disclosure provides an isolated nucleic acid molecule encoding the isolated proteins disclosed herein.

Also provided herein is a method of controlling plant pathogenic fungi comprising administering the isolated protein as disclosed herein to a subject or composition in need thereof, such as a plant or an animal. In one embodiment, the plant pathogenic fungus is one of a Fusarium spp., a Sclerotinia spp., a Rhizoctonia spp., or a Pythium spp. In an embodiment, the composition in need thereof is soil.

Further provided herein is a composition comprising the isolated protein disclosed herein, and a carrier and optionally further comprising an additional antifungal agent. In one embodiment, the present disclosure provides a composition comprising a polypeptide molecule comprising an amino acid sequence as shown in SEQ ID NO:36, SEQ ID NO:37, SEQ ID NO:38 or SEQ ID NO:39 or a variant thereof and a carrier and optionally further comprising an additional antifungal agent. In an embodiment, the composition is a seed treatment composition. In another embodiment, the composition is a plant treatment composition. In yet another embodiment, the composition is a soil treatment composition. Also provided herein is a method of controlling plant pathogenic fungi, such as a Fusarium spp., a Sclerotinia spp., a Rhizoctonia spp., or a Pythium spp. fungus, comprising administering the composition disclosed herein to a subject in need thereof. In one embodiment, the subject is a plant. In another embodiment, the subject is an animal. In an embodiment, the method comprises treating seeds, treating plants and/or treating plant growing substrates, such as soil.

Also provided herein are microbial cells, CHO cells, and other prokaryotic or eukaryotic cells transformed to produce one or more of the novel intracellular proteins and novel extracellular proteins. In one embodiment, the present disclosure provides a transformed microbial cell expressing a polypeptide molecule comprising an amino acid sequence as shown in SEQ ID NO:36, SEQ ID NO:37, SEQ ID NO:38 or SEQ ID NO:39 or a variant thereof.

The transformed microbial cells CHO cells, and other prokaryotic or eukaryotic cells can be used to control plant pathogenic fungi exemplified by Fusarium spp., Sclerotinia spp., Rhizoctonia spp., Pythium spp., and the like. The transformed microbial cells can be used as seed treatments and/or plant treatments and/or soil treatments, or any combination thereof. Alternatively, the transformed microbial cells can be used to produce the novel proteins. The proteins can be harvested and used to prepare the compositions disclosed herein. Further provided herein is a microbial cell transformed with a nucleic acid encoding an isolated protein disclosed herein. In one embodiment, the microbial cell is a fungal cell, a yeast cell or a bacterial cell. The fungal cell optionally is a Sphaerodes mycoparasitica cell or a Penicillium spp. cell. The yeast cell is optionally is a Zygosaccharomyces spp. cell, a Saccharomyces spp. cell, a Pichia spp. cell, or a Kluveromyces spp. cell. The bacterial cell is optionally an Escherichia coli cell, a Pseudomonas spp. cell, or a Bacillus spp. cell or a Methylobacterium spp. cell. The CHO cell is optionally a Chinese Hamster Ovary cell.

Further provided herein are transformed plant cells, transformed using a gene gun or any other known method to genetically modify plants, that express one or more of the novel proteins disclosed herein. In one embodiment, the present disclosure provides a transformed plant cell expressing a polypeptide molecule comprising an amino acid sequence as shown in SEQ ID NO:36, SEQ ID NO:37, SEQ ID NO:38 or SEQ ID NO:39 or a variant thereof. In one embodiment, the plant cell is resistant to infection by plant pathogenic fungi, such as Fusarium spp., Sclerotinia spp., Rhizoctonia spp., Pythium spp., and the like.

Such transformed plant cells can be comprised in plant propagules exemplified by seeds, somatic embryos, organogenic tissues and the like, that can be cultured into whole plants. Whole plants comprising the transformed plant cells are resistant to, or at least tolerant, of disease symptoms caused by plant pathogenic fungi exemplified by Fusarium spp., Sclerotinia spp., Rhizoctonia spp., Pythium spp., and the like. The plant cell is optionally an oil seed plant cell, a grain plant cell, a fibre plant cell, or a pulse plant cell.

In yet a further embodiment, the present disclosure provides a method for testing a sample of plant seeds for the presence therein of aurofusarin, the method comprising:

processing a portion of the sample of plant seeds to produce a DNA sample therefrom; and

processing the DNA sample with a PCR primer set comprising SEQ ID NO: 32 and SEQ ID NO: 33 to detect the presence and/or expression therein of a gene or nucleic acid sequence coding for aurofusarin. Also provided herein is an isolated nucleic acid molecule comprising a nucleotide sequence set forth in SEQ ID NO: 32 or SEQ ID NO: 33.

### BRIEF DESCRIPTION OF THE DRAWINGS

FIG. 1 is a micrograph showing Sphaerodes mycoparasitica cultures after two weeks of incubation on (A) Modified Leonian's agar and (B) Potato dextrose agar—upper sides, (C) and (D)—down sides;

FIG. 2 is a micrograph showing Sphaerodes mycoparasitica ascospore germination, showing single-polar and double-polar germination patterns as well as hyphal anastomosis (arrow) formation pattern;

FIG. 3 is a micrograph showing Sphaerodes mycoparasitica: (A) Ascoma, (B) Hyaline seta arising from the neck (arrow), (C) Reticulate ascospores (arrows), (D) Smooth ascospore (arrow), (E) Triangular ascospore (arrow), (F) Phialides produced on ascoma surrounding hyphae, (G) Ampulliform phialide arising from the surface of ascoma peridial wall, (H) Formation of mature and starting ascomata, (I) Formation of hook-like structures by S. mycoparasitica parasitising on living hypha of Fusarium oxysporum (arrows) and (J) Large view of hook-like structure on living hypha of F. avenaceum. Bar scales are 50 pm for (A) and (H); 10 pm for (B), (C), (D), (E), (F), (G) and (J); and 25 pm for (I);

FIG. 4(A) is a micrograph showing Sphaerodes mycoparasitica ascospores showing the conspicuous wall ornamentation and prominent irregular longitudinal ribs (arrows); (B) shows Sphaerodes quadrangularis ascospores. Bar scales are 5 pm;

FIG. 5(a) is a micrograph showing inhibition of mycelial growth of (A) Fusarium oxysporum and (B) Fusarium graminearum by total extracellular proteins recovered from a Sphaerodes mycoparasitica culture; 5(b) is a chart showing the percent inhibition of mycelial growth of A) Fusarium oxysporum and (B) Fusarium graminearum by S. mycoparasitica extracellular proteins;

FIG. 6 is a FPLC chromatogram showing the f1 and f2 extracellular proteins recovered from a Sphaerodes mycoparasitica culture;

FIG. 7 is micrograph of SDS-PAGE gels of purified extracellular proteins recovered from a Sphaerodes mycoparasitica culture. Lane M contains marker proteins. Lane 1 contains the purified protein from peak f1. Lane 2 contains the purified protein from peak f2;

FIG. 8 is micrographs showing the inhibition of F. oxysporum spore germination (A (a) and (b)) and F. graminearum spore germination (B (a) and (b)) by purified proteins f1 (a) and f2 (b) compared to controls (A (c) and B (c));

FIG. 9 is a chart showing the level of 3-ADON degradation by Sphaerodes mycoparasitica in potato-dextrose broth analysed by HPLC. Different letters above bars indicate significant differences in DON concentration (Kuskal-Wallis, P≦0.05);

FIG. 10 is a schematic illustration of a system for growing wheat plants in a container (4×4×16 cm) with different layers of soil-less growing mixes;

FIG. 11 is a micrograph of a gel showing SmyITSF/R primers amplified PCR products for S. mycoparasitica (SM), five Fusarium strains (Fa=F. avenaceum, Fo=F. oxysporum, Fs=F. sporotrichioides, Fg3=F. graminearum chemotype 3, and Fg15=F. graminearum chemotype 15), two Trichoderma species (T22=T. harzianum T22 and Tv=T. viride), two Cladosporium species ACC=C. cladosporioides and CM=C. minourae), and Penicillium aurantiogriseurn (PA) were electrophoresed on 1% agarose gel at 100 V for 20 minutes. The size of the band is around 300 to 400 bp;

FIG. 12 are charts showing standard linear curves for (A) Sphaerodes mycoparasitica (in the range of 3.8×10² to 3.8×10⁻² ng in ten-fold decreasing manner); (B) Fusarium graminearum 3-ADCIN (in the range of 2.7×10³ to 2.7×10⁻¹ ng in ten-fold decreasing manner); (C) Trichoderma harzianum T-22 (in the range of 7.0×10² to 7.0×10⁻² ng in ten-fold increasing manner);

FIG. 13 is a chart showing RT-PCR sigmoidal coloured curves for Sphaerodes mycoparasitica (SMCD 2220-01), with 0.025 fluorescence line, in the ranges of 3.8×10² to 3.8×10⁻² ng in a ten-fold decreasing manner;

FIG. 14 is charts showing quantities of genomic DNAs for (A) F. graminearum (Fgra); (b) S. mycoparasitica (SM); and (C) Trichoderma harzanium (T-22) monitored in spring wheat roots using genus-specific quantification real-time PCR. All values were means of 6 replicates. Error bars indicate SD;

FIG. 15 is a chart showing real-time fluorescence curves of tri5 gene sequences amplified by using Tox5-1/2 primer set from total DNA extracted from dual-culture assays of Fusarium graminearum strains and pre-inoculated Sphaerodes mycoparasitica SMCD2220-01 (SNI) or singly grown F. graminearum 3-ADON and 15-ADON chemotypes;

FIG. 16 is micrographs of Sphaerodes mycoparasitica ascomata and ascospores: (A) formation of S. mycoparasitica ascomata on a colony of F. avenaceum (Arrows indicate that ascomata were produced near or surrounding the Fusarium culture); (B) production of numerous S. mycoparasitica ostiolated perithecia being produced on a F. oxysporum colony (Arrows indicate pericthecia were formed on the Fusarium isolate; (C) ungerminated dark-brownish reticulated S. mycoparasitica ascospore; (D) germinating ascospore of S. mycoparasitica in F. oxysporum-filtrate suspension showing one polar germ pore: (E) single polar germinating spore of S. mycoparasitica after 3 d suspension in a F. oxysporum-filtrate; (F) ascospore of S. mycoparasitica illustrating two polar germination in F. oxysporum-filtrate suspension after 3 d with an additional 1 d on PDA; (G) pattern of S. mycoparasitica two polar germination in F. avenaceum-filtrate suspension with additional 1 d on PDA: (H) single polar germination in S. mycoparasitica spore suspended 3 d in F. avenaceum-filtrate with 1 d incubation on PDA; (I) single and double polar germinations demonstrated by S. mycoparasitica ascospores after 3 d incubation in a F. avenaceum-filtrate suspension plus 1 additional day incubation on PDA. Scale bars for (C) to (H) are 10 μm and for (I) is 20 μm. SM, Fa, and Fo represent S. mycoparasitica, F. avenaceum, and F. oxysporum, respectively;

FIG. 17 is a chart showing spore germination patterns of Sphaerodes mycoparasitica biotrophic mycoparasitic fungus with spores isolated from a F. oxysporum colony. Single (▪) and two (□) polar germination in a F. avenaceum-filtrate suspension; and Single ( [symbol] ) and two (▪) polar germination in a F. oxysporum-filtrate suspension. 1=1 d suspension, 2=1 d suspension plus 1 d on PDA incubation, 3=3 d suspension, and 4=3 d suspension plus 1 d on PDA incubation. Each incubation day for S. mycoparasitica was analyzed separately;

FIG. 18 is a chart showing germination of Sphaerodes mycoparasitica ascospores in filtrates of six Fusarium strains and water suspension treatments at four different incubation days. Suspension in different treatments were: Fave-filtrate=F. avenaceum-filtrate; Foxy-filtrate=F. oxysporum-filtrate; Fgra3-filtrate=F. graminearum chemotype 3 filtrate; Fgra15-filtrate=F. graminearum chemotype 15 filtrate; Fpro-filtrate=F. proliferatum filtrate; Fspo-filtrate=F. sporotrichioides filtrate; and water=control. Day of incubations were: 1 d suspension=spores suspended for 1 d in different suspension treatments; 1 d sus+PDA=spores suspended for 1 d in suspension treatment and then inoculated onto PDA medium for an additional day; 3 d suspension=spores suspended for 3 d in different suspension treatments; and 3 d sus+PDA=spores suspended for 3 d in suspension treatment and then inoculated onto PDA medium for an additional day. Different letters above bars indicate significant differences (Kuskal-Wallis, P≦0.05);

FIG. 19 is charts showing linear mycelial growths of (Top) Fusarium graminearum chemotype 3, and (Bottom) Fusarium graminearum chemotype 15 in dual-culture assays challenged with Sphaerodes mycoparasitica in co-inoculation (same day) ( [symbol] ) and pre-inoculation (1 day prior to Fusarium inoculation) ( [symbol] ) treatments as well as control (without mycoparasite) ( [symbol] ) for 5 days;

FIG. 20 is micrographs of interactions between F. graminearum 3-ADON (Fgra3) and 15-ADON (Fgral 5) chemotypes on slide culture assays with S. mycoparasitica (S) biotrophic mycoparasitic fungus: (A) Single mycelium of S. mycoparasitica, (B) F. graminearum mycelium with red complex, (C) Absorption of red complex from F. graminearum by S. mycoparasitica (arrow), (D) Excretion of red complex in crystal-forms (arrows) by S. mycoparasitica from mycelium interacting with F. graminearum chemotype 3 only, (E) Formation of series of hook-like structures by S. mycoparasitica, (F) Parasitism of F. graminearum mycelium by S. mycoparasitica with formation of hook-shaped structures, excretion of crystal-like compounds (arrows), and internal haustorium, (G) Initiation of penetration-peg formation by S. mycoparasitica on F. graminearum, (H) Infected or penetrated and non-infected myclial cells, (I) Branching of haustorium inside Fusarium host, (J) Formation of extensive short branching structures by F. graminearum chemotype 15 only at the contact zone with S. mycoparasitica. Bar scales: (A) to (I) is 5 μm, and (J) is 20 μm;

FIG. 21 is a chart showing the differences in diameters between infected and non-infected F. graminearum chemotype 3 (Fgra3) and 15 (Fgral 5) host cells in presence of Sphaerodes mycoparasitica on slide culture assays. Bars demarked by different lowercase letters represent significant difference in size of infected vs. non-infected hyphae for the two different F. graminearum chemotypes at P=0.05 using a T-test;

FIG. 22 is charts of the standard curves of Fusarium graminearum chemotype 3-ADON and 15-ADON genomic DNA concentration standards versus cycle threshold (Ct) with PCR reactions performed in triplicate using primer sets; (A) Tox5-1/2, with genomic DNA ranging from 270 ng (Log₁₀=2.90) to 0.27 ng (Log₁₀=−0.60), readings at 0.005 fluorescence line; (B) Tox5-1/2, with genomic DNA ranging from 30 ng (Log₁₀=1.48) to 0.03 ng (Log₁₀=−1.52), readings at 0.005 fluorescence line; and (C) Fg16NF/R, with DNA template ranging from 270 ng (Log₁₀=2.43) to 0.027 ng (Log₁₀=−1.57); in 10-fold dilution series, readings at 0.025 fluorescence line. Error bars indicate standard deviation for the mean of F. graminearum chemotype 3-ADON and 15-ADON standard curves derived from tri5 gene and F. graminearum specific primer set;

FIG. 23 is a schematic illustration of the experimental set-up for dual-culture assays used to acquire F. graminearum chemotype 3-ADON or 15-ADON mycelial plugs for DNA extraction. The sampling zone (S-zone) indicates the 0.5×1.5 cm² sample area situated approximately 0.2 cm behind the interaction zone (1-zone). The I-zone represents the interaction or contact zone between F. graminearum (Fgra) and S. mycoparasitica (SM);

FIG. 24 is a chart showing real-time fluorescence curves of F. graminearum sequences amplified using Fg16NF/R primer set from total DNA extracted from dual-culture assays of F. graminearum strains and pre-inoculated Sphaerodes mycoparasitica (SM), or singly grown F. graminearum cultures of chemotype 3-ADON and 15-ADON;

FIG. 25 is a chart showing comparisons between different concentrations of DNA from F. graminearum chemotype 3-ADON and 15-ADON amplified with Tox5-1/2 (tri5 gene specific) and Fg16NF/R (F. graminearum-specific) primer sets. Fungal DNAs were extracted from 5 d dual-culture assays pre-inoculated with S. mycoparasitica for 1 d. With T-test at P=0.05, for the comparison between S. mycoparasitica treated and non-treated F. graminearum chemotype 3-ADON and 15-ADON for Tox5-1/2 and Fg16NF/R primer sets, respectively. (Log10 transformed for DNA amplified with Tox5-1/2 primers); Different letters above bars indicate significant differences (Kuskal-Wallis, P≦0.05)

FIG. 26 is micrographs of Fusarium graminearum mycelia at the contact zone with biological and chemical agents: (A) No visible cell changes with Sphaerodes mycoparasitica biotrophic mycoparasite; (B) Cell abruption with Trichoderma harzianum necrotrophic mycoparasite; (C) 3-ADON chlamydospores formation in chains when challenged with fungicide; (D) 15-ADON chlamydospores formation in clusters when challenged with fungicide. Scale bars indicate: (A)=5 μm and (B)-(D)=10 μm;

FIG. 27 is charts showing gene expression of different Tri genes for F. graminearum 3-ADON chemotype and F. graminearum 15-ADON chemotype (in in vitro assays with three separate treatments: (A) Tri4 gene; (B) Tri5 gene; (C) Tri6 gene; and (D) Tri10 gene. Legends: B3=S. mycoparasitica+3-ADON producing F. graminearum; F3=Folicur+3-ADON producing F. graminearum; T3=T. harzianum+3-ADON producing F. graminearum; B15=S. mycoparasitica+15-ADON producing F. graminearum; F15=Folicur+15-ADON producing F. graminearum; and T15=T. harzianum+15-ADON producing F. graminearum. Means for three different treatments in F. graminearum chemotype 3-ADON and 15-ADON were analyzed separately with LSD test. Different letters above bars indicate significant differences at P=0.05. Values are means±SE of three samples;

FIG. 28 is charts showing gene expression of PKS genes for F. graminearum 3-ADON chemotype and F. graminearum 15-ADON chemotype in in vitro assays with three separate treatments: (A) PKS4 and (B) PKS13. Legends: B3=S. mycoparasitica+3-ADON producing F. graminearum; F3=Folicur+3-ADON producing F. graminearum; T3=T. harzianum+3-ADON producing F. graminearum; B15=S. mycoparasitica +15-ADON producing F. graminearum; F15=Folicur+15-ADON producing F. graminearum; and T15=T. harzianum+15-ADON producing F. graminearum. Means for three different treatments in F. graminearum chemotype 3 and 15 were analyzed separately with LSD test. Different letters above bars indicate significant differences at P=0.05;

FIG. 29 is a micrograph of thin liquid chromatography (TLC) analysis for zearalenone (ZEA) extracted from six separate treatments. Legends: B3=S. mycoparasitica+3-ADON producing F. graminearum; F3=Folicur+3-ADON producing F. graminearum; T3=T. harzianum+3-ADON producing F. graminearum; F15=Folicur+15-ADON producing F. graminearum; T15=T. harzianum+15-ADON producing F. graminearum, B15=S. mycoparasitica+15-ADON producing F. graminearum, and ZEA=Standard of zearalenone;

FIG. 30 is a chart showing the ratio of F. graminearum chemotype challenged with Folicur fungicide to F. graminearum alone control produced for all four different mycotoxins—ZEA, DON, 3ADON and 15ADON. Legends indicate F. graminearum 3-ADON chemotype with Folicur (▪) and F. graminearum 15-ADON chemotype with Folicur (□);

FIG. 31 is a chart showing relative AUR gene expression in Fusarium strains after co-culturing with biological and chemical agents. Legend: 15-ADON—F. graminearum 15-acetyl-deoxynivalenol chemotype; 3-ADON—F. graminearum 15-acetyl-deoxynivalenol chemotype; F.cul.—F. culmorum; F.ave.—F. avenaceum; B—Sphaerodes mycoparasitica (biotrophic mycoparasite); Tricho—Trichoderma harzianum (necrotrophic mycoparasite); Fol—Folicur (tebuconazole) fungicide;

FIG. 32 is a chart showing changes in AUR gene expression evidenced by the color of fungal hyphae. Legend: Ds #71232B: Red and highly virulent, tolerant at 80° C. for 4 hours; Es #4E040B: Moderately red and moderately virulent, tolerant 40° C. for 4 hours; Bs #A86608: White and non virulent, susceptible 40° C. for 4 hours;

FIG. 33 is charts showing the effects of inoculating a Fusarium-susceptible barley cultivar with F. graminearum (Fg only), S. mycoparasitica (Sm only), and treating F. graminearum-infected barley with different concentrations (10⁴, 10⁵, and 10⁶ CFU per mL) on: (A) height of the plants; (b) average number of spikes per plant; and (C) the average weight of 5 barley spikes; Different letters above bars indicate significant differences (Kuskal-Wallis, P≦0.05);

FIG. 34 is charts showing the effects of inoculating a Fusarium-susceptible wheat cultivar with F. graminearum (Fg only), S. mycoparasitica, (Sm only), and treating F. graminearum-infected wheat with different S. mycoparasitica concentrations (10⁴, 10⁵, and 10⁶ CFU per mL) on: (A) height of the plants; (B) average number of spikes per plant; and (C) the average weight of 5 barley spikes; Different letters above bars indicate significant differences (Kuskal-Wallis, P≦0.05);

FIG. 35 is a chart comparing the biocontrol effects of S. mycoparasitica (B) on the severity of Fusarium head blight symptoms with the protection provided by the commercial fungicide Folicur (Fol). Treatments were Fusarium only (Fus), S. mycoparasitica+Fusarium (B-Fus), Folicur+Fusarium (Fol-Fus).

FIG. 36 are charts showing standard curves of Fusarium graminearum chemotype 3-ADON genomic DNA concentration standards versus cycle threshold (Ct) with PCR reactions performed in triplicate using primer sets: (A) Tox5-1/2, with genomic DNA ranging from 270 ng (Log₁₀=2.90) to 0.27 ng (Log₁₀=−0.60), readings at 0.005 fluorescence line; and (B) Fg16NF/R, with DNA template ranging from 270 ng (Log₁₀=2.43) to 0.027 ng (Log₁₀=−1.57); in 10-fold dilution series, readings at 0.025 fluorescence line;

FIG. 37 is a chart showing the effects of S. mycoparasitica (B) and Folicur fungicide (Fol) treatments on F. graminearum chemotype 3-ADON genomic DNA detected in barley spikes employing RT-PCR. Treatments were: Fus—F. graminearum; B-Fus—S. mycoparasitica with F. graminearum; Fol-Fus—Folicur fungicide with F. graminearum;

FIGS. 38A and 38B are micrographs showing Sphaerodes mycoparasitica-Fusarium spp. mycoparasitism assays: (A and a). Hook-shaped contact structures (arrows); (B and b). Clamp-like clasping cells (arrows). “a” and “b” are diagrammatic drawings for (A) and (B) respectively. Scale bars=5 μm;

FIG. 39 is micrographs showing intracellular parasitism, hyphal inhibition response, and anamorphic stages during the Sphaerodes mycoparasitica-Fusarium spp. interactions: (A) Intracellular parasitism by S. mycoparasitica in F. equiseti (arrow); (B) Fusarium hyphal inhibition response when challenged with S. mycoparasitica; deformation of hyphae into rosette-like shapes (arrow); (C) Hyaline S. mycoparasitica anamorphic stages; (D) Sphaerodes mycoparasitica anamorphic stages with adsorption of red pigments from F. culmorum. Scale bars A, C, D=5 μm; B=20 μm;

FIGS. 40A and 40B are micrographs of intracellular parasitism by Sphaerodes mycoparasitica inside F. equiseti (arrows), and 40C and 40D are micrographs of intracellular hyphae produced by Sphaerodes mycoparasitica inside F. equiseti with hook-shaped contact structure (arrows). 40A and 40C were captured under light microscopy; whereas in 40B and 40D hyphae were stained with lactofuchsin and images were captured under confocal laser microscopy and fluorescent microscopy, respectively. Scale bars=5 μm; and

FIG. 41 is a chart showing average hyphal diameters of parasitized and non-parasitized F. equiseti cells (▪) and F. culmorum (□) on 1-week slide-cultures with Sphaerodes mycoparasitica biotrophic mycoparasite. Data are means and standard deviations. Same lowercase letters indicate no significant difference between parasitized and non-parasitized hyphae at P=0.05, with T-test.

FIGS. 42(a)A and 42(a)B are micrographs of a disc diffusion assay showing the inhibitory effects of exemplary intracellular proteins of the present disclosure on mycelial growth of Fusarium oxysporum (42(a)A) and F. graminearum (42(a)B), and FIG. 42(b) is a chart showing the % inhibition of mycelial growth of F. oxysporum (A) and F. graminearum (B) by the exemplary proteins after 3 days and 4 days of growth;

FIG. 43 is a micrograph of 10% native-PAGE separation of intracellular proteins produced by Sphaerodes mycoparasitica into two bands;

FIG. 44 is a micrograph of 12% SDS-PAGE separation of S. mycoparasitica intracellular proteins eluted from the upper band and lower band indicated on the 10% native-PAGE gel shown in FIG. 43;

FIG. 45 is a micrograph of 10% native-PAGE separation of extracellular proteins produced by S. mycoparasitica into four bands;

FIG. 46 is a micrograph of 12% SDS-PAGE separation of S. mycoparasitica extracellular proteins eluted from the four bands of the 10% native-PAGE gel shown in FIG. 45;

FIG. 47 is a chromatogram showing FLPC separation of the four bands of extracellular proteins produced by S. mycoparasitica;

FIG. 48(a) is a micrograph of mycelial growth of F. oxysporum in the control treatment; 48(b) is a micrograph of mycelial growth of F. oxysporum in the control treatment plus buffer; 48(c) is a micrograph of the inhibitory effects of the 50 kDa intracellular protein on mycelial growth of F. oxysporum; and 48(d) is a micrograph of the inhibitory effects of the 79 kDa intracellular protein on mycelial growth of F. oxysporum;

FIG. 49(a) is a micrograph of mycelial growth of F. graminearum in the control treatment; 49(b) is a micrograph of mycelial growth of F. graminearum in the control treatment plus buffer; 49(c) is a micrograph of the inhibitory effects of the 50 kDa intracellular protein on mycelial growth of F. graminearum; and 49(d) is a micrograph of the inhibitory effects of the 79 kDa intracellular protein on mycelial growth of F. graminearum;

FIG. 50(a) is a micrograph showing the inhibition of mycelial outgrowth of F. oxysporum by the 79 kDa extracellular protein (EC1) compared to the control treatment (C), and 50(b) is a micrograph showing the inhibition of mycelial outgrowth of F. oxysporum by the 50 kDa extracellular protein (EC2) and the 36 kDa extracellular protein (EC3);

FIG. 51(a) is a micrograph showing the inhibition of mycelial outgrowth of F. graminearum by the 79 kDa extracellular protein (EC1) compared to the control treatment (C), and 51(b) is a micrograph showing the inhibition of mycelial outgrowth of F. graminearum by the 50 kDa extracellular protein (EC2) and the 36 kDa extracellular protein (EC3);

FIG. 52(a) is a micrograph showing the inhibition of mycelial outgrowth of F. graminearum by the 13 kDa extracellular protein (EC4) compared to the control treatment (C), and 52(b) is a micrograph showing the inhibition of mycelial outgrowth of F. oxysporum by the 13 kDa extracellular protein (EC4) compared to the control treatment (C);

FIGS. 53(a) and 53(b) are charts showing the effects of S. mycoparasitica extracellular protein fractions EC1, EC2, EC3, and EC4 on germination of F. oxysporum spores 53(a) and F. graminearum spores 53(b);

FIG. 54 is a chart showing the effects of S. mycoparasitica extracellular protein fractions EC1, EC2, EC3, and EC4 on inhibition of colony growth and development of F. oxysporum and F. graminearum;

FIGS. 55A(a), 55A(b), and 55A(c) are micrographs showing the inhibitory effects of the 50 kDa protein on germination of F. oxysporum spores (a), the inhibitory effects of the 13 kDa protein on germination of F. oxysporum spores (b), and germination of control F. oxysporum spores (c), and FIGS. 55B(a), 55B(b), and 55B(c) are micrographs showing the inhibitory effects of the 50 kDa protein on germination of F. graminearum spores (a), the inhibitory effects of the 13 kDa protein on germination of F. graminearum spores, and germination of control F. graminearum spores (c);

FIGS. 56(a) and 56(b) are micrographs showing the effects of crystal-forming extracellular proteins EC1 and EC3 on mycelial growth of F. avenaceum (56(b)) in comparison to the control (56(a));

FIGS. 57(a) and 57(b) are micrographs showing the effects crystal-forming extracellular proteins EC1 and EC3 on the colonization of germinating wheat seeds and mycelial growth of F. graminearum (57(b)) in comparison to the control (57(a)), while 57(c) is a confocal laser scanning microscopy micrograph showing numerous lysed F. graminearum hyphal elements (see arrows) from the surface of the wheat seedlings treated with the extracellular proteins. “K+” indicates that the medium contained the EC1 and EC3 extracellular proteins. “K−” indicates that the EC1 and EC3 extracellular proteins were absent from the medium. “K-crystal” identifies the location of an extracellular protein molecule(s);

FIGS. 58(a) and 58(b) are micrographs showing the effects of the crystal-forming extracellular proteins EC1 and EC3 on mycelial growth of F. graminearum (58(b)) in comparison to the control (58(a));

FIG. 59(a) is a scanning electron microscopy micrograph showing the presence of protein crystals and lysed fungal hyphae from the crystal-protein-treated F. graminearum from FIG. 58(b), 59(b) is a confocal laser scanning microscopy micrograph showing the presence of protein crystals among the hyphae of the crystal-protein-treated F. graminearum from FIGS. 58(b), and 59(c) is a chemical force microscopy micrograph showing the presence of protein crystals and numerous broken hyphal elements and apoptotic-lysed cells (arrows) from the crystal-protein-treated F. graminearum from FIG. 58(b). “K crystal: indentifies the location of an extracellular protein molecule(s)”;

FIG. 60 is a chart showing MALDI-TOF-MS separation of amino acids of the 79 kDA EC1 protein;

FIG. 61 is a chart showing MALDI-TOP-MS separation of amino acids of the 50 kDa EC2 protein;

FIG. 62 is a chart showing MALDI-TOF-MS separation of amino acids of the 36 kDa EC3 protein;

FIG. 63 is a chart showing MALDI-TOF-MS separation of amino acids of the 13 kDa EC3 protein;

FIGS. 64(a)-64(f) are micrographs of: (64(a)) a control untreated culture of Sclerotinia sclerotiorum on PDA, (64(b)) a S. sclerotiorum culture grown on PDA amended with extracellular proteins EC1 plus EC3, (64(c)) a control untreated culture of Rhizoctonia solani, (64(d)) a R. solani culture grown on PDA amended with extracellular proteins EC1 plus EC3, (64(e)) a control untreated culture of Pythium ultimum, and (64(f)) a P. ultimum culture grown on PDA amended with extracellular proteins EC1 plus EC3; and

FIG. 65 is a chart showing the inhibitory effects of the crystal-forming extracellular proteins EC1 and EC3 on mycelial growth of Sclerotinia sclerotiorum, Rhizoctonia solani, and Pythium ultimum.

### DETAILED DESCRIPTION

### Strains

Sphaerodes mycoparasitica (Ascomycetes, Melanosporales) is a mycoparasite that has been isolated from isolates of Fusarium avenaceum, Fusarium graminearum and Fusarium oxysporum originating from wheat or asparagus fields. The species is characterized by a unique combination of ascospore size, shape (fusiform and triangular) and wall ornamentation (reticulate and smooth). Also, conidia are produced from simple phialides on the surface of ascoma peridial wall, on ascoma surrounding hyphae, and on irregularly branched conidiophores arising from hyphae. S. mycoparasitica has a phialidic anamorph and produces simple phialides on the surface of ascoma peridial wall or scattered irregularly on ascoma surrounding the hyphae, and on conidiosphores. S. mycoparasitica forms hook-like structures parasitizing living hyphae of Fusarium.

Accordingly, the present disclosure provides an isolated culture of Sphaerodes mycoparasitica, wherein the species is characterized by a combination of:

(a) fusiform, triangular, reticulate and smooth;

(b) producing simple phialides on the surface of ascoma perdial wall or scattered irregularly on ascoma surrounding the hyphae, and on conidiosphores; and

(c) forming hook-like structures parasitizing living hyphae of Fusarium.

Sample cultures of Sphaerodes mycoparasitica have been deposited with Saskatchewan Microbial Collection and Database under the accession number SMCD2220-01, and in the International Depositary Authority of Canada Collection (1015 Arlington Street, Winnipeg, Canada, R3E 3R2) under the accession number IDAC301008-01. Sample cultures of Sphaerodes mycoparasitica biotrophically parasitizing Fusarium avenaceum (teleomorph: Giberella avenacea) have been deposited with Saskatchewan Microbial Collection and Database under the accession number SMCD2220-02, and with the International Depositary Authority of Canada Collection under the accession number IDAC301008-02, and is referred to herein as “SM-Bst”. Sample cultures of Sphaerodes mycoparasitica biotrophically parasitizing Fusarium graminearum (telemorph: Giberella zeae) have been deposited with Saskatchewan Microbial Collection and Database under the accession number SMCD2220-03, and with the International Depositary Authority of Canada Collection under the accession number IDAC301008-03, and is referred to herein as “SM-Gst”.

Accordingly, the present disclosure also provides an isolated culture of Sphaerodes mycoparasitica strain IDAC 301008-01. The present disclosure further provides an isolated culture of Sphaerodes mycoparasitica strain from IDAC 301008-02. The present disclosure additionally provides an isolated culture of Sphaerodes mycoparasitica from IDAC strain 301008-03.

The 1266 bp DNA sequence from the large subunit ribosomal RNA gene (LSU) of S. mycoparasitica is given in SEQ ID NO: 1.

| SEQ ID NO: 1 | |
|---|---|
| 1 | atagggagaa gaagcactgc gattgcccta gtaacggcga gtgaagcggc agcagcccag |
| 61 | atttggaatc tggtcctttt ggggcccgag ttgtaatctg cagaggaagc gtctggtgcg |
| 121 | gtgccggcct agttccctgg aacgggacgc cgtagagggt gacagccccg tacggtcggc |
| 181 | caccaaacct gtgtgtcgct ccttcgaaga gtcgcgtagt ttgggaatgc tgcgtaaagt |
| 241 | gggaggtatg ctcctcctaa ggctaaatac cggccagaga ccgatagcgc acaagtagag |
| 301 | tgatcgaaag atgaaaagca ccttgaaaat ggggttaaaa agtacgtgaa attgccaaag |
| 361 | gggaagcgct cgtggccaga ctcgtgcctt atggatcatc cggctatttc gccggtgcac |
| 421 | tccattaggc tcgggccagc gtcggtcggc gccggtacta aaagacagcg cgaacgtggc |
| 481 | tctcttcggg gagtgttata gcgcgctgtg taatgtgctg gcgccgtccg aggaccgcgc |
| 541 | atttatgcaa ggacgctggc gtaatggcca ctagcgaccc gtcttgaaac acggaccaag |
| 601 | gagtcgccca gagacgcgag tgtgcgggtg acaaacccct gcgcgaagtg aaagcgaacg |
| 661 | ctggtgggaa ccctcacggg tgcaccaccg accgatcctg atgtcttcgg atggatttga |
| 721 | gtatgagcgt ttctggtcgg acccgaaaga gggtgaacta tgcttgggta gggtgaagcc |
| 781 | agaggaaact ctggtggagg ccccgtttgg gttctgacgt gcaaatcgat ccataaacct |
| 841 | gggcatagcg gcgaaagact aatcgaacct tctagtagct ggttcgcatt ctctctctcg |
| 901 | cacgagagag agaaaacctc tgtgatatca cgattatcag tgaaaaccac accgagaccc |
| 961 | aacggagttc ttctggattt cctcatgctt caattaccac gcctagtgga cctacctgga |
| 1021 | gcgctacaat aaagtcatac gaaaatctcg aagatcgggg tgacggtgag ggatcctaag |
| 1081 | gttctctcgt tgagtgcgtt ggacgggcat ggccgtcagc gatctggggc gaccgttgcc |
| 1141 | ggatcataag ggctttagtg cttaggctat tggtattgag gggtctgaag acggtaatct |
| 1201 | gaaaccaaag gctttattct aaacccgcgc agcatgggcg tagtaggaag agacagcgaa |
| 1261 | gtctag. |

The 1233 bp DNA sequence from the small subunit ribosomal RNA gene (SSU) of S. mycoparasitica is given in SEQ ID NO: 2.

| SEQ ID NO: 2 | |
|---|---|
| 1 | agtgcggcat gttgtagcct aagcaattat acagcgaaac tgcgaatggc tcattatata |
| 61 | agttatcgtt tatttgatag tgccttacta cttggataac cgtggtaatt ctagagctaa |
| 121 | tacatgctga aagccccgac ttacggaggg gcgtatttat tagattaaaa accaatgccc |
| 181 | ttcggggctc tttggtgatt catgataact tctcgaatcg cacggccttg cgccggcgat |
| 241 | ggttcattca aatttcttcc ctatcaactt tcgatgtttg ggtagtggcc aaacatggtg |
| 301 | gcaacgggta acggagggtt agggctcgac cccggagaag gagcctgaga aacggctcct |
| 361 | acatccaagg aaggcagcag gcgcgcaaat tacccaatct caactcgagg aggtagtgac |
| 421 | aataaatacc gatgcagggc tctttagggt cttgcaattg gaatgagtac aatttaaatc |
| 481 | ccttaacgag gaacaattgg agggcaagtc tggtgccagc agccgcggta actccagctc |
| 541 | caatagcgta tattaaagtt gttgtggtta aaaagctcgt agttgaacct tgggcctggc |
| 601 | cggctggtcc gcctaacagc gtgcactggt gcggccgggt cttcccaccg cggagccgca |
| 661 | tgtccttcac tgggcgtgtc ggggaagcgg tacttttact gtgaaaaaat tagagtgctc |
| 721 | taagcaggcc tatgctcgaa tacattagca tggaataata gaataggaca gtcgttctat |
| 781 | tttgttggtt tctaggacgt ctgtaatgat taacagaaac aatcgggggc gtcagtattg |
| 841 | catcgtcaga ggtgaaattc ttagatcgat gcaagactaa ctactgcgaa agcattcgcc |
| 901 | aagggtgttt tcattaatca ggaacgaaag ttaggggatc gaagacgatc agataccgtc |
| 961 | gtagtcttaa ccataaacta tgccgactag ggatcgggcg gtgtaatttt gacccgctcg |
| 1021 | gcacttacga gaaatcttaa gtgcttgggc tccaggggag tatggtcgca aggctgaaac |
| 1081 | ttaaagaaat gacgaagggc accaccaagg gtgaacctgc ggcctagttg actcacacgg |
| 1141 | gaaactcacg aggtaggaat atgtagatga cggatggagc cttcagaata catatgggca |
| 1201 | tgcgctctta ataccggtat tgaaaatggg cag |

Internal Transcribed Spacer ribosomal DNA from S. mycoparasitica was sequenced. A 560 bp DNA sequence from the ITS region is given in SEQ ID NO: 3.

| SEQ ID NO: 3 | |
|---|---|
| 1 | ttcgtttctt atcgcattgg tgaccagcgg agggtcatta cgaatcggac catttatgtc |
| 61 | atggctctgc caaccctgtg aactttatac ttgtacgttg cctcggcgga acctgccttt |
| 121 | tcggcaggcc gccggccggc atatacgcaa acgctctgaa aaagctccgc gctctatctg |
| 181 | aataataaaa ctttaacgag taaaaacttt tggcaacgga tctcttggct ctggcatcga |
| 241 | tgaaaaacgc agcgaaatgc gatacgtaat gtgaattgca gaattcagtg aaccatcgaa |
| 301 | tctttgaacg caccttgcgg ccgccggtaa tccggcggcc atgcccgtcc gagcgtcgtt |
| 361 | tccaccctcg ggagttctcc tcctaagaaa atttctcccg gccttgggcc agcgcgttgc |
| 421 | gcggctgccc gaccaacggc ggcaggaccg gcgatgtcct ctgtgccctg catttatata |
| 481 | aaactcgcat tggtccccgg taaggcttgc cttgcaacca acttctttag gtcgacctca |
| 541 | gatcggatag ggatacccgc |

Accordingly, the present disclosure provides an isolated culture of Sphaerodes mycoparasitica comprising a gene encoding a large subunit of ribosomal RNA gene as shown in SEQ ID NO:1 or a variant thereof. Also provided herein is an isolated culture of Sphaerodes mycoparasitica comprising a gene encoding a small subunit of ribosomal RNA as shown in SEQ ID NO:2 or a variant thereof. Further provided herein is an isolated culture of Sphaerodes mycoparasitica comprising a gene encoding an internal transcribed spacer ribosomal DNA as shown in SEQ ID NO:3 or a variant thereof.

In the context of this specification, a “conserved” variant describes a sequence that has similarity with the reference sequence. The degree of conservation between two sequences can be determined by optimally aligning the sequences for comparison. Sequences may be aligned using the Omiga software program, Version 1.13. (Oxford Molecular Group, Inc., Campbell, Calif.). The Omiga software uses the Clustal W Alignment algorithms [Higgins et a1.2007, Mycological Research 111: 509-547; Thompson et al. 1994, Nucleic Acids Research 24: 4876-4882]. Default settings used are as follows: Open gap penalty 10.00; Extend gap penalty 0.05; Delay divergent sequence 40 and Scoring matrix—Gonnet Series. Percent identity or homology between two sequences is determined by comparing a position in the first sequence with a corresponding position in the second sequence. When the compared positions are occupied by the same nucleotide or amino acid, as the case may be, the two sequences are conserved at that position. The degree of conservation between two sequences is often expressed as a percentage representing the ratio of the number of matching positions in the two sequences to the total number of positions compared.

The disclosure further encompasses variants that differ from any of the nucleic acid molecules of the disclosure in codon sequences due to the degeneracy of the genetic code.

Further, it will be appreciated that the disclosure includes nucleic acid molecules comprising nucleic acid sequences having substantial sequence identity with the nucleic acid sequence as shown in SEQ ID NOS: 1-3 or fragments thereof. The term “sequences having substantial sequence identity” means those nucleic acid sequences that have slight or inconsequential sequence variations from these sequences, i.e., the sequences function in substantially the same manner. The variations may be attributable to local mutations or structural modifications.

Nucleic acid sequences having substantial identity include nucleic acid sequences having at least about 50 percent identity with a protein encoded by SEQ ID NOS: 1-3. The level of sequence identity, according to various aspects of the disclosure is at least about: 60, 70, 75, 80, 83, 85, 88, 90, 93, 95 or 98 percent. Methods for aligning the sequences to be compared and determining the level of homology between the sequences are described in detail above.

Sequence identity can be calculated according to methods known in the art and as detailed below.

The present disclosure also provides a primer set, SmyITSF/R (SmyITSF is SEQ ID NO: 4 and SmyITSR is SEQ ID NO: 5), useful for identifying S. mycoparasitica:

SEQ ID NO: 4
5′-TCATGGCTCTGCCAACCCTGTAA-3′
SEQ ID NO: 5
5′-AATGCAGGGCACAGAGGACATCG-3′

The present primer set is selective for S. mycoparasitica and can be used to assess and quantify the fungus in industrial products, plant materials, seed samples, and environmental samples by using PCR and RT-PCR technologies.

The SmyITSF/R primer set can be used for quantitative real-time PCR technology for analyzing gene expression, in fungal pathogens detection, and in quantification of fungi in living plants.

The SmyITSF/R primer set was tested with SMCD2220-01, seven Fusarium species, nine different ascomycetous fungal isolates, two zygomycete fungi, and three basidiomycetous fungal strains.

In PCR, this primer set only amplified SMCD2220-01/IDAC301008 01, not the other fungi. Root biomass, total biomass, root length, total length, and seed germination of F. graminearum infected spring wheat were significantly increased with the treatments of S. mycoparasitica, as compared to inoculation with F. graminearum. In further RT-PCR studies, SmyITSF/R specific primer was used for S. mycoparasitica in combination with F. graminearum-Fg12NF/R and Trichoderma-TGP4-F/R as a control, within which the primer showed high accuracy in assessing biocontrol-pathogen-plant interactions.

In the present disclosure, S. mycoparasitica may be isolated from suitable sources. For example, S. mycoparasitica may be isolated from F. graminearum or F. avenaceum isolates originating from wheat fields by the method described in Vujanovic V, et. al. Can. J. Microbiol. 48(9): 841-847 (2002). S. mycoparasitica culture is available deposited with International Depositary Authority of Canada (IDAC301008-01) and in co-culture with Fusarium avenaceum (IDAC301008-02) and Fusarium graminearum (IDAC301008-03).

S. mycoparasitica may be produced via fermentation and formulated into a pesticide composition. A suitable fermentation process includes, selection of fermentation medium (solid state or submerged), concentration of fermentation constituents, oxygen transfer, incubation temperature, time of harvest and post harvest treatments. The aim is to establish the optimum conditions to ensure an abundant, stable, compatible, and efficacious mycoparasitic microbial population. When formulating S. mycoparasitica into a pesticide composition, care should be taken to selected ingredients that: (i) ensure stability during production, processing and storage, (ii) assist application, (iii) protect the pesticide from unfavourable environmental conditions, and (iv) promote pesticidal activity at the target. Exposure to inactivated host/pathogen or its compounds may improve the activity of S. mycoparasitica.

### Proteins, Nucleic Acids, Vectors and Host Cells

Total extracellular protein extracts and intracellular protein extracts from Sphaerodes mycoparasitica demonstrated significant inhibition of the germination of fungal spores of Fusarium spp., Sclerotinia spp., Rhizoctonia spp., and Pythium spp. Accordingly, the present disclosure provides an antifungal agent comprising extracellular protein extracts and intracellular protein extracts from S. mycoparasitica. Four proteins were isolated from the total protein extract, one having a molecular weight of 13 kDa and comprising a partial sequence as shown in SEQ ID NO:39; one having a molecular weight of 36 kDa and comprising a partial sequence as shown in SEQ ID NO:38; one having a molecular weight of 50 kDa and comprising a partial sequence as shown in SEQ ID NO:37; and one having a molecular weight of 79 kDa and comprising a partial sequence as shown in SEQ ID NO:36. The present disclosure provides an antifungal agent comprising one or more of these proteins.

Conventional molecular biological techniques may be used to isolate, characterize and produce the presently disclosed proteins. For example, in order to identify the gene(s) for the present proteins, S. mycoparasitica could be challenged with F. oxysporum filtrates and upregulated mRNA isolated by standard Northern Blot. cDNA can be produced from the mRNA by Reverse Transcriptase PCR (RT-PCR). The cDNA can be amplified, purified and inserted into an appropriate vector. This vector may be inserted into an appropriate host cell.

Cloning and expression may focus on isolation of genes coding for antimicrobial proteins by designing primers for identified proteins. Isolated genes may be cloned in suitable expression vectors (yeast or bacteria) with suitable/efficient promoters in order to generate industrial strains for large-scale production of the concerned proteins. These vectors can be purchased from Promega, Invitrogen, Clontech, or other companies. The cloned genes may be tested for their protein expression, and the expressed proteins will be purified (using His-tag or other columns). Purified proteins may be tested against plant-pathogenic fungi for their antifungal activity by disc-plate assay and/or microtitre plate assay methods (e.g., Drummond and Waigh, 2000, Recent Research Developments in Phytochemistry. 4:143-152). Then, rDNA technologies, involving gene mutation or addition of enhancers, introns or protein-specific promoters (GA inducible), or addition deletion may be used to enhance the production of proteins.

The selected genes encoding for one or more or all of the novel proteins, i.e., the 13 kDa protein, the 36 kDa protein, the 50 kDa protein, and the 79 kDa protein, may be transformed into selected plant genomes to create transgenic lines with antifungal activity against pathogenic fungi using techniques known to those skilled in these arts such as those exemplified by Sanghyun et. al. (2008, Transgenic wheat expressing a barley class II chitinase gene has enhanced resistance against Fusarium graminearum. J. Expt. Botany, 59, 2371-2378) and Tobias et. al. (2007, Co-bombardment, integration and expression of rice chitinase and thaumatin-like protein genes in barley (Hordeum vulgare cv. Conlon). Plant Cell Reports 26: 631-639). Suitable crop plants for transformation with these novel proteins are exemplified by oil seed crops, grain crops, fibre crops, pulse crops, coniferous tree species, and deciduous tree species among others. Exemplary oil seed crops include canola, mustard, rapeseed, soybean, flax, sunflower, safflower, castor bean, and camelina among others. Exemplary grain crops include wheat, barley, oats, triticale, rye, rice, maize, sorghum, and quinoa, among others. Exemplary fibre crops include cotton, jute, flax, hemp, and bamboo among others. Exemplary pulse crops include beans, peas, chickpeas, lentils, and lupins among others. Exemplary coniferous tree species include, pine, spruce, fir, and hemlock, among others. Exemplary deciduous tree species include poplar, aspen, maple, oak, nut-producing trees, fruit trees, acacia, and eucalyptus among others.

It will be appreciated that the disclosure includes amino acid molecules comprising amino acid sequences having substantial sequence identity with the amino acid sequences shown in SEQ ID NOs: 36-39. The term “sequences having substantial sequence identity” means those amino acid sequences that have slight or inconsequential sequence variations from these sequences, i.e., the sequences function in substantially the same manner to produce functionally equivalent proteins. The variations may be attributable to local mutations or structural modifications.

Amino acid sequences having substantial identity include amino acid sequences having at least about 50 percent identity with a protein having an amino acid sequence as shown in SEQ ID NOs:36-39. The level of sequence identity, according to various aspects of the disclosure is at least about: 60, 70, 75, 80, 83, 85, 88, 90, 93, 95 or 98 percent. Methods for aligning the sequences to be compared and determining the level of homology between the sequences are described in detail above.

Sequence identity can be calculated according to methods known in the art. Sequence identity is most preferably assessed by the algorithm of BLAST version 2.1 advanced search. BLAST is a series of programs that are available, for example, online from the National Institutes of Health. The advanced blast search is set to default parameters. (i.e. Matrix BLOSUM62; Gap existence cost 11; Per residue gap cost 1; Lambda ratio 0.85 default). References to BLAST searches are: Altschul, S. F., Gish, W., Miller, W., Myers, E. W. & Lipman, D. J. (1990) “Basic local alignment search tool.” J. Mol. Biol. 215:403410; Gish, W. & States, D. J. (1993) “Identification of protein coding regions by database similarity search.” Nature Genet. 3:266272; Madden, T. L., Tatusov, R. L. & Zhang, J. (1996) “Applications of network BLAST server” Meth. Enzymol. 266:131_{—}141; Altschul, S. F., Madden, T. L., Schäffer, A. A., Zhang, J., Zhang, Z., Miller, W. & Lipman, D. J. (1997) “Gapped BLAST and PSI_BLAST: a new generation of protein database search programs.” Nucleic Acids Res. 25:33893402; Zhang, J. & Madden, T. L. (1997) “PowerBLAST: A new network BLAST application for interactive or automated sequence analysis and annotation.” Genome Res. 7:649656.

Analogs of the proteins having the amino acid sequences shown in SEQ ID NOs:36-39 as described herein, may include, but are not limited to an amino acid sequence containing one or more amino acid substitutions, insertions, and/or deletions. Amino acid substitutions may be of a conserved or non-conserved nature. Conserved amino acid substitutions involve replacing one or more amino acids of the proteins of the disclosure with amino acids of similar charge, size, and/or hydrophobicity characteristics. When only conserved substitutions are made the resulting analog should be functionally equivalent. Non-conserved substitutions involve replacing one or more amino acids of the amino acid sequence with one or more amino acids which possess dissimilar charge, size, and/or hydrophobicity characteristics.

Conservative substitutions are described in the patent literature, as for example, in U.S. Pat. No. 5,264,558. It is thus expected, for example, that interchange among non-polar aliphatic neutral amino acids, glycine, alanine, proline, valine and isoleucine, would be possible. Likewise, substitutions among the polar aliphatic neutral amino acids, serine, threonine, methionine, asparagine and glutamine could possibly be made. Substitutions among the charged acidic amino acids, aspartic acid and glutamic acid, could probably be made, as could substitutions among the charged basic amino acids, lysine and arginine. Substitutions among the aromatic amino acids, including phenylalanine, histidine, tryptophan and tyrosine would also likely be possible. Other substitutions might well be possible.

One or more amino acid insertions may be introduced into the amino acid sequences shown in SEQ ID NOs: 36-39. Amino acid insertions may consist of single amino acid residues or sequential amino acids ranging from 2 to 15 amino acids in length. Such variant amino acid molecules can be readily tested for competitive inhibition with the target protein or protein transport activity.

Deletions may consist of the removal of one or more amino acids, or discrete portions from the amino acid sequence shown in SEQ ID NOs:36-39. The deleted amino acids may or may not be contiguous.

Exemplary methods of making the alterations set forth above are disclosed by Sambrook et al (Molecular Cloning: A Laboratory Manual, 2nd Ed., Cold Spring Harbor Laboratory Press, 1989).

The proteins described above (including truncations, analogs, etc.) may be prepared using recombinant DNA methods. These proteins may be purified and/or isolated to various degrees using techniques known in the art. Accordingly, nucleic acid molecules having a sequence which encodes a protein of the disclosure may be incorporated according to procedures known in the art into an appropriate expression vector which ensures good expression of the protein. Possible expression vectors include but are not limited to cosmids, plasmids, or modified viruses (e.g., replication defective retroviruses, adenoviruses and adeno-associated viruses), so long as the vector is compatible with the host cell used. The expression “vectors suitable for transformation of a host cell”, means that the expression vectors contain a nucleic acid molecule encoding a peptide of the disclosure and regulatory sequences, selected on the basis of the host cells to be used for expression, which are operatively linked to the nucleic acid molecule. “Operatively linked” is intended to mean that the nucleic acid is linked to regulatory sequences in a manner which allows expression of the nucleic acid.

The disclosure further contemplates a recombinant expression vector of the disclosure containing a nucleic acid molecule that encodes a protein of the disclosure and the necessary regulatory sequences for the transcription and translation of the inserted protein-sequence.

The recombinant expression vectors of the disclosure may also contain a selectable marker gene that facilitates the selection of host cells transformed or transfected with a recombinant molecule of the disclosure. Examples of selectable marker genes are genes encoding a protein which confers resistance to certain drugs, such as G418 and hygromycin.

Recombinant expression vectors can be introduced into host cells to produce a transformed host cell. The term “transformed host cell” is intended to include prokaryotic and eukaryotic cells which have been transformed or transfected with a recombinant expression vector of the disclosure. The terms “transformed with”, “transfected with”, “transformation” and “transfection” are intended to encompass introduction of nucleic acid (e.g. a vector) into a cell by one of many possible techniques known in the art.

Suitable host cells include a wide variety of prokaryotic and eukaryotic host cells. For example, the proteins of the disclosure may be expressed in bacterial cells such as E. coli, insect cells (using baculovirus), yeast cells, plant cells or mammalian cells, COS1 cells. Other suitable host cells can be found in Goeddel, Gene Expression Technology: Methods in Enzymology 185, Academic Press, San Diego, Calif. (1991).

Alternatively, the proteins can be prepared by chemical synthesis using techniques well known in the chemistry of proteins such as solid phase synthesis [Merrifield 1964 J. Am. Chem. Assoc. 85, 2149-2154] or synthesis in homogeneous solution [Houbenwycl, 1987 Methods of Organic Chemistry, Vol I and II].

Accordingly, in another embodiment, the selected genes encoding for one or more or all of the novel proteins, i.e. the 13 kDa protein, the 36 kDa protein, the 50 kDa protein, and the 79 kDa protein, may be transformed into selected host cells to create transgenic lines with antifungal activity against pathogenic fungi using techniques known to those skilled in these art.

Accordingly, there is provided a microbial cell transformed to produce one or more of the novel intracellular proteins and novel extracellular proteins. In one embodiment, the present disclosure provides a transformed microbial cell expressing a polypeptide molecule comprising an amino acid sequence as shown in SEQ ID NO:36, SEQ ID NO:37, SEQ ID NO:38 or SEQ ID NO:39 or a variant thereof.

The transformed microbial cells can be used to control plant pathogenic fungi exemplified by Fusarium spp., Sclerotinia spp., Rhizoctonia spp., Pythium spp., and the like. The transformed microbial cells can be used as seed treatments and/or plant treatments and/or soil treatments, or any combination thereof. Alternatively, the transformed microbial cells can be used to produce the novel proteins. The proteins can be harvested and used to prepare the compositions disclosed herein. Further provided herein is a microbial cell transformed with a nucleic acid encoding an isolated protein disclosed herein. In one embodiment, the microbial cell is a fungal cell, a yeast cell or a bacterial cell. The fungal cell optionally is a Sphaerodes mycoparasitica cell or a Penicillium spp. cell. The yeast cell is optionally is a Zygosaccharomyces spp. cell, a Saccharomyces spp. cell, a Pichia spp. cell, or a Kluveromyces spp. cell. The bacterial cell is optionally an Escherichia coli cell, a Pseudomonas spp. cell, or a Bacillus spp. cell.

Further provided herein are transformed plant cells that express one or more of the novel proteins disclosed herein. In one embodiment, the present disclosure provides a transformed plant cell expressing a polypeptide molecule comprising an amino acid sequence as shown in SEQ ID NO:36, SEQ ID NO:37, SEQ ID NO:38 or SEQ ID NO:39 or a variant thereof. In one embodiment, the plant cell is resistant to infection by plant pathogenic fungi, such as Fusarium spp., Sclerotinia spp., Rhizoctonia spp., Pythium spp., and the like.

Such transformed plant cells can be comprised in plant propagules exemplified by seeds, somatic embryos, organogenic tissues and the like, that can be cultured into whole plants. Whole plants comprising the transformed plant cells are resistant to, or at least tolerant of, disease symptoms caused by plant pathogenic fungi exemplified by Fusarium spp., Sclerotinia spp., Rhizoctonia spp., Pythium spp., and the like. The plant cell is optionally an oil seed plant cell, a grain plant cell, a fibre plant cell, or a pulse plant cell.

### Compositions

The present disclosure provides antifungal compositions comprising S. mycoparasitica, isolates, cultures, or proteins thereof.

In one embodiment, the compositions disclosed herein comprise (i) an active ingredient (the S. mycoparasitica, isolates, cultures or proteins thereof), (ii) carriers—often an inert material used to support and deliver the densely populated active ingredient to the target, and optionally (iii) adjuvants—compounds that; Promote and sustain the function of the active ingredient by protection from UV radiation; Ensure rain fastness on the target; Retain moisture or protect against desiccation; and/or Promote the spread and dispersal of the biopesticide using standard agriculture equipments such as those disclosed by Hynes and Boyetchko (2006, Soil Biology & Biochemistry 38: 845-84).

In one embodiment, the present disclosure provides antifungal compositions comprising one or more or all of the 79 kDa protein (comprising SEQ ID NO:36), the 50 kDa protein (comprising SEQ ID NO:37), the 36 kDa protein (comprising SEQ ID NO:38), and the 13 kDa protein (comprising SEQ ID NO:39) produced by S. mycoparasitica. Alternatively, one or more or all of the 13 kDa protein, the 36 kDa protein, the 50 kDa protein, and the 79 kDa protein may be produced by transformed microorganisms provided with one or more nucleic acid molecules encoding the one or more or all of the 13 kDa protein, the 36 kDa protein, the 50 kDa protein, and the 79 kDa protein. The present disclosure also provides a method of controlling fungi, such as controlling germination, growth, division and infections or fungal disease of a plant or animal, and a method for mycotoxin detoxification, which methods comprise administering to the plant or animal S. mycoparasitica, isolates, cultures, or proteins thereof, such as the 13 kDa protein, the 36 kDa protein, the 50 kDa protein, and the 79 kDa protein, collectively hereinafter referred to as the ‘antifungal agent’. In one embodiment, administering to the plant comprises applying to the locus of the plant. In another embodiment, administering to the plant comprises treating soil with the antifungal agent.

The compositions of the disclosure may, for example, be applied to the seeds or propagules of the plants, to the growth medium (e.g. soil or water), to the roots of plants and/or to the foliage of the plants, or to any combination thereof. Exemplary crop plants that can be treated with the present compositions include agricultural crops such as seed crops, grain crops, fibre crops, pulse crops, horticultural crops, forestry crops, and turf grasses.

The present disclosure provides a composition comprising the antifungal agent and an agriculturally acceptable carrier or diluent, which will ensure stability and performance of the final product. The carrier or diluent should be compatible with the active ingredient, agriculturally acceptable, have a good absorptive capacity and a suitable bulk density, allowing easy particle dispersion and attachment.

The compositions herein may be applied, as in aqueous sprays, granules and dust/powder formulations in accordance with established practice in the art. An aqueous spray is usually prepared by mixing a wettable powder or emulsifiable concentrate formulation of a compound of the disclosure with a relatively large amount of water to form a dispersion.

Wettable powders may comprise a finely divided mixture of the antifungal agent, a solid carrier, and a surface-active agent. The solid carrier is usually chosen from among attapulgite clays, kaolin clays, montmorillonite clays, diatomaceous earths, finely divided silica, purified silicates, or combinations thereof. Surfactants which may be useful herein have wetting, penetrating, and/or dispersing ability. They are typically present in an amount of from about 0.5% to about 10% by weight. Surfactants herein may be chosen from, for example, alkylbenzenesulfonates, alkyl sulfates, naphthalenesulfonates and condensed naphthalenesulfonates, sulfonated lignins, and non-ionic surfactants.

Emulsifiable concentrates may comprise the antifungal agent of the disclosure in a liquid carrier, the carrier being a mixture of a water-immiscible solvent and a surfactant. Solvents that may be useful herein include aromatic hydrocarbon solvents such as the xylenes, alkylnaphthalenes, petroleum distillates, terpene solvents, ether-alcohols, organic ester solvents or suitable combinations thereof.

When a composition of the disclosure is to be applied to plant debris or litter, in order to control of the source of contamination and inoculant dispersion, or to the soil, as for pre-emergence protection, granular formulations or dusts are sometimes more convenient than sprays.

In one embodiment the antifungal agents herein are encapsulated into alginate pellets. The pellets may be prepared in any suitable manner. For example, one useful method is described in Harveson et. al., (2002, Plant Disease; Vol. 86, No. 9 1025-1030).

In another embodiment, the present disclosure provides a composition comprising the antifungal agent and a pharmaceutically acceptable carrier or diluent. The pharmaceutical compositions can be prepared by known methods for the preparation of pharmaceutically acceptable compositions which can be administered to patients, and such that an effective quantity of the active substance is combined in a mixture with a pharmaceutically acceptable vehicle. The pharmaceutically acceptable vehicle may be chosen to permit administration by oral, topical, transmucosal, injection, inhalation routes, or by any other known route Suitable vehicles and dosage forms are described, for example, in Remington's Pharmaceutical Sciences (Remington's Pharmaceutical Sciences, Mack Publishing Company, Easton, Pa., USA 2003-20th Edition) and in The United States Pharmacopeia (The National Formulary (USP 24 NF19) published in 1999).

On this basis, the pharmaceutical compositions include, albeit not exclusively, the active compound or substance in association with one or more pharmaceutically acceptable vehicles or diluents, and contained in buffered solutions with a suitable pH and iso-osmotic with the physiological fluids. In one embodiment, the pharmaceutically acceptable vehicle or diluent is sterile phosphate buffered saline, sterile saline, or purified water.

The composition may comprise other active substances useful in an antifungal agent. For example, the compositions herein may comprise other antifungal agents such as Trichoderma, sulfur, neem oil, rosemary oil, jojoba oil, Bacillus subtilis, allylamines (e.g. terbinafine, antimetabolites (e.g. flucytosine), azoles (e.g. ketoconazole, itraconazole), echinocandins (e.g. caspofungin), polyenes (e.g. amphotericin B), systemic agents (e.g. griseofluvin), or combinations thereof.

The compositions herein may contain from about 0.1% to about 95%, by weight, of the antifungal agent and from about 0.1% to about 95%, by weight, of the carrier and/or surfactant. The direct application to plant seeds prior to planting may be accomplished in some instances by mixing either a powdered solid compound of the disclosure or a dust formulation with seed to obtain a substantially uniform coating which is very thin and represents only one or two percent by weight or less, based on the weight of the seed. In some instances, however, a non-phytotoxic solvent such as methanol is conveniently employed as a carrier to facilitate the uniform distribution of the compound of the disclosure on the surface of the seed.

### Methods and Uses

S. mycoparasitica, isolates, cultures, proteins and compositions disclosed herein may be used to treat, ameliorate, or otherwise control plant pathogenic fungi, such as Fusarium, Scleritinia, Rhizoctonia and Pythium. S. mycoparasitica, isolates, cultures and proteins and compositions disclosed herein may be applied in any suitable manner to the organism in need of treatment. S. mycoparasitica, isolates, cultures and proteins and compositions disclosed herein may also be applied to soil. For example, S. mycoparasitica, isolates, cultures and proteins and compositions disclosed herein may be used directly or they may be further processed into inoculants for application to soils systems or soil-less growing systems (e.g. granular compositions, peat-based compositions), for application to seeds (e.g., powdered compositions, granular compositions, peat-based compositions, liquid compositions), or application to growing plants (e.g., powdered compositions, liquid-based compositions).

Sphaerodes mycoparasitica, isolates, cultures and proteins and compositions disclosed herein may be useful as an anti-fungal agent. S. mycoparasitica, isolates, cultures and proteins and compositions disclosed herein may be used to treat, ameliorate, or otherwise control Fusarium fungi, Sclerotinia fungi, Rhizoctonia fungi or Pythium fungi. S. mycoparasitica, isolates, cultures and proteins and compositions disclosed herein seem particularly useful for treating, ameliorating or otherwise control Fusarium avenaceum, Fusarium graminearum, and/or Fusarium oxysporum and improving plant health or growth or treating fungal infections in animals. The term “animal” as used herein includes all members of the animal kingdom including vertebrates, mammals, and humans.

The phrase “control fungi” as used herein refers to controlling or inhibiting germination, growth, division and/or controlling or treating diseases or infections caused by the fungi.

The term “treatment or treating” as used herein means an approach for obtaining beneficial or desired results, including clinical results. Beneficial or desired results can include, but are not limited to, alleviation or amelioration of one or more symptoms or conditions, diminishment of extent of disease, stabilized (i.e. not worsening) state of disease, preventing the disease or condition or preventing the spread of the disease or condition, delay or slowing of disease progression, amelioration or palliation of the disease state, and remission (whether partial or total), whether detectable or undetectable. “Treating” can also mean prolonging survival as compared to expected survival if not receiving treatment.

S. mycoparasitica, isolates, cultures and proteins and compositions disclosed herein may be used as a prophylactic agent to diminish the chance of a fungal infection, particularly a Fusarium Sclerotinia, Rhizoctonia, or Pythium infections, from occurring.

S. mycoparasitica, isolates, cultures and proteins and compositions disclosed herein may be used for treating plants affected by Fusarium Wilt Disease or Fusarium Head Blight. S. mycoparasitica, isolates, cultures, proteins and compositions disclosed herein may also be used for treating food and feed affected by Fusarium or mycotoxins. S. mycoparasitica, isolates, cultures, proteins and compositions disclosed herein may be used for hindering such conditions from spreading or as a prophylactic measure to diminish the risk of such conditions and contaminants from occurring in natural or industrial/processing samples.

The formulations disclosed herein comprise (i) one or more active ingredients comprising one or more of the novel proteins, (ii) one or more carriers—often an inert material used to support and deliver the densely populated active ingredient to the target, and optionally (iii) one or more adjuvants-compounds that; Promote and sustain the function of the active ingredient by protection from UV radiation; Ensure rain fastness on the target; Retain moisture or protect against desiccation; and/or Promote the spread and dispersal of the biopesticide using standard agriculture equipments such as those disclosed by Hynes and Boyetchko (2006, Research initiatives in the art and science of biopesticide formulations. Soil Biol. & Biochem. 38: 845-49).

S. mycoparasitica has demonstrated an ability to degrade the deoxynivalenol (DON) mycotoxin. Accordingly, the present disclosure also provides a method of degrading or modulating synthesis of acetyldeoxynivalenol, such as 3-acetyldeoxynivalenol comprising administering antifungal compositions comprising S. mycoparasitica, isolates, culture or proteins thereof, such as the 13 kDa protein, the 36 kDa protein, the 50 kDa protein, and the 79 kDa protein disclosed herein.

The present inventors have demonstrated that S. mycoparasitica significantly decreases

DON-mycotoxin concentration through the detoxification/degradation of mycotoxin in contaminated samples, for example, as shown in Example 3. Accordingly, in another embodiment, the present disclosure provides a method for detoxifying food, feed, or an environmental sample comprising one or more of a Fusarium trichothecene mycotoxin deoxynivalenol (DON), mycotoxin 3-ADON, mycotoxin 15-ADON, mycotoxin zerelanone, and mycotoxin aurofusarin comprising administering the culture of Sphaerodes mycoparasitica disclosed herein to said food, feed, or environmental sample.

The term “environmental sample” as used herein refers includes, without limitation, natural or industrial samples including water, soil, organic material, minerals, metals, industrial processing equipment, shipping containers, and the like.

S. mycoparasitica could be challenged with pure DON and upregulated mRNA isolated by standard Northern Blot. Reverse Transcriptase PCR could be used to identify the gene(s) for the upregulated proteins, an appropriate cDNA construct could then be inserted into an appropriate vector for protein production. Further, recombinant DNA Technology could be applied to create transgenic plants with antimycotoxin properties.

S. mycoparasitica sporulates in the presence of Fusarium. S. mycoparasitica ascospores proved resistant to germination under different standard laboratory conditions (sterile distilled water, on water agar and commercially available media) and heat or cold-shock treatments. In contrast, spore germination was obtained on general potato dextrose agar medium amended with Fusarium-filtrates. Significant improvement in percentage of spore germinations were obtained for the spores suspended in Fusarium-filtrates. F. avenaceum and F. oxysporum filtrates induced the highest germination, whereas F. sporotrichioides and F. proliferatum triggered lower germination frequency. Filtrates of beneficial fungal inoculants: Trichoderma harzianum (RootShield® available from BioWorks Inc., Victor, N.Y., USA; RootShield is a registered trademark of BioWorks Inc.), Penicillium bilaii (JumpStart® available from Novozymes Biologicals Ltd., Saskatoon, SK, CA; JumpStart is a registered trademark of Philom Bios Inc.), and Chaetomium globosum had no impact on germination. Ascospores suspended in F. avenaceum-filtrate, showed double-polar germination pattern. On the other hand, when suspended in F. oxysporum-filtrate, significant amount of spores demonstrated a single-polar germination pattern. S. mycoparasitia grown on F. oxysporum kept the same mycoparasitic germination patterns in three offspring generations when transferred on F. avenaceum which indicate a stable genome-regulated expression.

Accordingly, the present disclosure provides a method of sporulating S. mycoparasitica by exposing them to F. avenaceum or F. oxysporum, or filtrates, extracts, or compositions thereof.

The present disclosure also provides a method for producing an antifungal composition comprising S. mycoparasitica or isolates, cultures, genes or proteins thereof, the method comprising:
(a) inducing S. mycoparasitica sporulation;
(b) culturing S. mycoparasitica;
(c) harvesting S. mycoparasitica.
   The method may further comprise any of the following optional steps:
(d) storing/preserving S. mycoparasitica;
(e) applying S. mycoparasitica;
(f) assessing S. mycoparasitica;
(g) producing S. mycoparasitica proteins;
(h) harvesting S. mycoparasitica proteins;
(i) fractionating S. mycoparasitica proteins;
(j) separating S. mycoparasitica proteins;
(k) storing/preserving S. mycoparasitica proteins;
(l) applying S. mycoparasitica proteins;
(m) assessing S. mycoparasitica proteins.

In one embodiment, the present disclosure provides a method for producing an antifungal composition comprising one or more or all of the S. mycoparasitica 13 kDa protein, the 36 kDa protein, the 50 kDa protein, and the 79 kDa protein, the method comprising:
(a) inducing sporulation of S. mycoparasitica spores;
(b) culturing S. mycoparasitica;
(c) recovering proteins from the S. mycoparasitica culture,
(d) separating and recovering one or more or all of the 13 kDa protein, the 36 kDa protein, the 50 kDa protein, and the 79 kDa protein from the recovered S. mycoparasitica proteins; and
(e) preparing a composition comprising one or more or all of the recovered 13 kDa protein, the 36 kDa protein, the 50 kDa protein, and the 79 kDa protein from the S. mycoparasitica proteins.

The 79 kDa protein (comprising SEQ ID NO:36), the 50 kDa protein (comprising SEQ ID NO:37), the 36 kDa protein (comprising SEQ ID NO:38), and the 13 kDa protein (comprising SEQ ID NO:39) may be separated and recovered together from the recovered S. mycoparasitica proteins. Alternatively, the 13 kDa protein and/or the 36 kDa protein and/or the 50 kDa protein and/or the 79 kDa protein, or any combination thereof may be recovered individually from the recovered S. mycoparasitica proteins.

The present disclosure further provides a method for producing an antifungal composition comprising one or more or all of the S. mycoparasitica 79 kDa protein (comprising SEQ ID NO:36), the 50 kDa protein (comprising SEQ ID NO:37), the 36 kDa protein (comprising SEQ ID NO:38), and the 13 kDa protein (comprising SEQ ID NO:39), the method comprising:
(a) transforming a selected microbial culture with one or more genes encoding for one or more or all of the 13 kDa protein, the 36 kDa protein, the 50 kDa protein, and the 79 kDa protein produced by S. mycoparasitica;
(b) culturing the transformed microbial culture;
(c) recovering proteins from the transformed culture,
(d) separating and recovering 13 kDa protein and/or the 36 kDa protein and/or the 50 kDa protein and/or the 79 kDa protein from the recovered proteins; and
(e) preparing a composition comprising one or more or all of the 13 kDa protein, the 36 kDa protein, the 50 kDa protein, and the 79 kDa protein.

The 13 kDa protein and/or the 36 kDa protein and/or the 50 kDa protein and/or the 79 kDa protein may be separated and recovered together from the recovered proteins. Alternatively, 13 kDa protein and/or the 36 kDa protein and/or the 50 kDa protein and/or the 79 kDa protein may be recovered individually from the recovered proteins. Suitable microbial cultures that may be transformed with one or more genes encoding for one or more or all of the 13 kDa protein, the 36 kDa protein, the 50 kDa protein, and the 79 kDa protein produced by S. mycoparasitica are exemplified by bacterial cultures such as Escherichia coli, Pseudomonas spp., Bacillus spp., and the like, yeast cultures such as Zygosaccharomyces spp., Saccharomyces spp., Pichia spp., Kluveromyces spp. and the like, and Penicillium spp. and the like.

The above disclosure generally describes the present application. A more complete understanding can be obtained by reference to the following specific examples. These examples are described solely for the purpose of illustration and are not intended to limit the scope of the disclosure. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

### Sampling, Fungal Growth and Microscopy

Myclobutanil-agar (MBA) medium was used for selective isolation of various Fusarium taxa and associated biotrophic mycoparasites from Canadian agriculture fields using the method described in Vujanovic V, et. al. (2002, Can. J. Microbiol. 48(9): 841-847). Sphaerodes was recovered occasionally from F. graminearum and abundantly from F. avenaceum isolates originating from wheat fields in Saskatchewan; it was also isolated from Fusarium oxysporum from asparagus fields in Quebec, Canada. A monosporal, single culture of the mycoparasite was obtained from each Fusarium species according to the method proposed by Harveson & Kimbrough (2001, Int. J. Plant Sci. 162(2):403-410). Single ascospore isolates were maintained on Potato dextrose agar (PDA) (Difco, BD Biosciences, Mississauga, ON, CA) supplemented with antibiotics (100 μg 1⁻¹ streptomycin sulphate and 13 μg 1⁻¹ neomycin sulphate; Sigma-Aldrich Canada Ltd., Oakville, ON, CA) and stored at −80° C. in Saskatchewan Microbial Collection and Database (SMCD2220-01) and in the International Depositary Authority of Canada (IDAC301008-01) collections. Fungal growth was assessed on modified Leonian's agar (MLA) and Potato dextrose agar (PDA) media. Biotrophic interactions between Sphaerodes and Fusarium strains were examined with the slide culture method proposed by Jordan & Barnett (1978, Mycologia 70(2):300-312). Morphological studies of ascomata, ascospores, mycelia, and anamorphic structures were performed after two weeks of incubation (21° C.-22° C.) under a Carl Zeiss Axioskop2 with a Carl Zeiss AxioCam ICc1 camera. Fungal materials for microscopic observation were mounted in lactofuchsin and lactophenol cotton blue dyes.

### DNA Extraction, Amplification and Sequencing

Three Sphaerodes strains: SMCD 2220-01 on F. avenaceum from wheat, SMCD 2220-02 on F. graminearum from wheat, and SMCD 2220-03 on F. oxysporum from asparagus were cultured on PDA medium at 21° C. for a week prior to DNA extraction. Genomic DNA was extracted with the DNeasy® Plant Mini Kit (Qiagen Inc., Mississauga, ON, CA; DNEasy is a registered trademark of Qiagen GmbH Corp, Hilden, Fed. Rep. Germany). LSU (large subunit) rDNA fragments were amplified using primer sets NS1/NS6 using techniques known to those skilled in these arts (e.g., Gardes & Bruns, 1993, Molecular Ecology 2: 113-118; White et al. 1990, PCR Protocols: a guide to methods and application: 315-322. Academic Press, New York) and LS1/LR5 (e.g., Hausner et al. 1993, Canadian Journal of Botany 71: 52-63; Rehner & Samuels, 1995, Canadian Journal of Botany 73 (Suppl. 1): S816-S823; Zhang & Blackwell, 2002, Mycological Research 106: 148-155). Target regions of fungal genomic DNA samples were amplified using polymerase chain reaction (PCR) in a 25 μl reaction mixture containing 2.5 μl of 10× buffer, 5 μl of Q buffer, 0.5 μl 10 mM dNTPs, 1 μl of each primer, 0.13 μl of 0.625 unit of Taq DNA Polymerase, 2 μl of extracted fungal DNA, and 12.87 μl of sterilized ultra-pure Millipore water. The Qiagen Taq PCR core kits were purchased from Qiagen Inc., Mississauga, ON, CA. Purified DNA PCR products were sequenced.

### Sequence Alignment and Phylogenetic Analyses

Sequences of LSU from this study and sequences retrieved from GenBank were aligned using Clustal X software (version 1.82) (Thompson et al. 1997, Nucleic Acids Research 24: 4876-4882), and edited in Bioedit (Hall, 1999, Nucleic Acids Symposium Series 41: 95-98). Distance trees were produced with PAUP (Phylogenetic Analysis Using Parsimony) 4.0b10 software (Swofford 2000, PAUP*. Phylogenetic Analysis Using Parsimony, Sinauer Associates, Sunderland, Mass.) using a neighbor-joining approach, and validated using bootstrap analyses with 1,000 repetitions. A fungal distance tree was prepared with sequences showing bootstrap values higher than 50%. Trees were rooted with sequences Xylaria hypoxylon U47841.

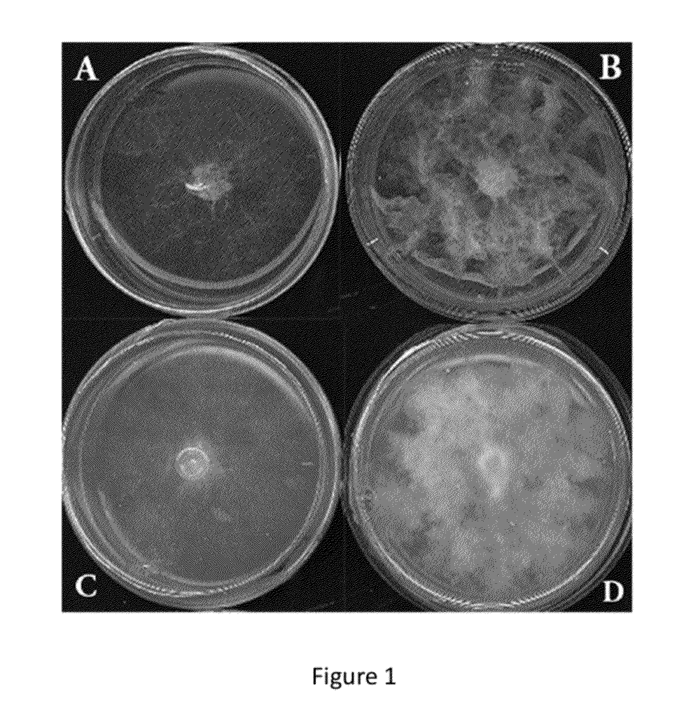

Taxonomy: Sphaerodes mycoparasitica Vujanovic, sp. nov. (FIGS. 1-5) [MycoBank no: MB 515144], in the International Depositary Authority of Canada as Sphaerodes mycoparasitica strain IDAC 301008-01.

Coloniae in agaro potato dextrosum lentior crescents, 4.0 cm ad 7d, floccose, pallido-brunneis. Hyphis septatis, ramosis, anastomosantibus, laevibus, palide fulvis, 2.5-5.0 μm diam compositum. Ascomata superficialia vel immersa, pyriformia vel globosa, ostiolata, flavo-brunnea, 250-300 μm longa, 200-280 μm diam. Collum nul, conicum vel cylindricum, 30-75 μm longum, (0-) 50-70 μm latum ad basim. Peridium membranaceum, cellulis 8-15 μm, e 3-6 stratis, 8-15 μm crassum, textura angulari compositum. Setae rectae vel parum curvae, hyalinae vel dilute flavae, crassitunicatae, 10-40 μm longae, septatae. Asci 8-spori, ovoidei vel clavati, 50-75×17-25 μm, superne late rotundati, brevistipitati, tenuitunicati, evanescentes. Paraphysis nullis. Ascosporae unicellulares, irregulariter biseriatae, primum hyalinae, deinde brunneae vel atrobrunneae, crassitunicatae, fusiformes, 18-24×9-12 μm, reticulatae, costis protrudentibus, e polo visae polygonales, utrinque umbonatae, foramine germinali praeditae. Phialidis hyalinis, status conidialis.

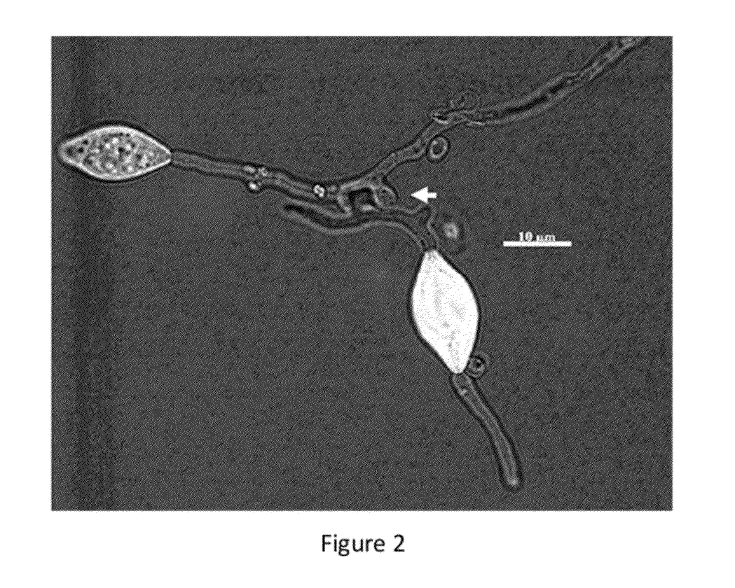

Culture characteristics: Colonies of Sphaerodes mycoparasitica strain IDAC 301008-01 cultured on MLA grew more rapidly than on PDA, 1.1 cm versus 0.6 cm per day (21-22° C.), consisting of slightly submerged mycelium and aerial hyphae, granulose due to production of ample number of ascomata. On MLA (FIGS. 1A and 1C) and PDA FIGS. 1B and 1D), the cultures produced a woolly mycelium, yellowish to pinky-brownish on both sides. At 37° C., no growth was registered. Hyphae were white to pale yellow, 2.5-5.0 μm diam., septate, anastomosis occurred soon after ascospore germination (FIG. 2). Colonies on Potato dextrose agar (PDA) spread with abundant, white to pale yellow aerial mycelium and low number of ascomata. Ascomata, perithecial or cleistotecial, scattered or aggregated in small groups, superficial, pyriform to globose, ostiolate (when mature), light to dark yellowish brown, translucent, appearing black due to mass of mature ascospores, 250-300 μm high, 200-280 μm diam. Neck absent to short conical or cylindrical 25-75 μm long, 20-70 gm wide at the base, sometimes surrounded with a crown of short, upright setae, 10-40 μm long. Peridium membranaceous, 3-6-layered, 8-10 μm thick, translucent, pale yellow to light brown, composed by cells of 8-15 μM diam. disposed in textura angularis. Asci 8-spored, clavate, 50-75×17-25 μm, rounded at apex, without apical structures, thin-walled and evanescent when mature. Paraphyses absent. Ascospores irregularly arranged inside the asci, at first hyaline but becoming brown to dark brown, thick-walled, single-celled, fusiform to rarely triangular, 18-24×9-12 μm, reticulate to rarely smooth, with irregular transverse sections, and with a strongly umbonate germ pore at each end. Phialides hyaline, ampulliform produced directly on ascomata or on hyphae surrounding ascomata, and on irregularly branched conidiophores.

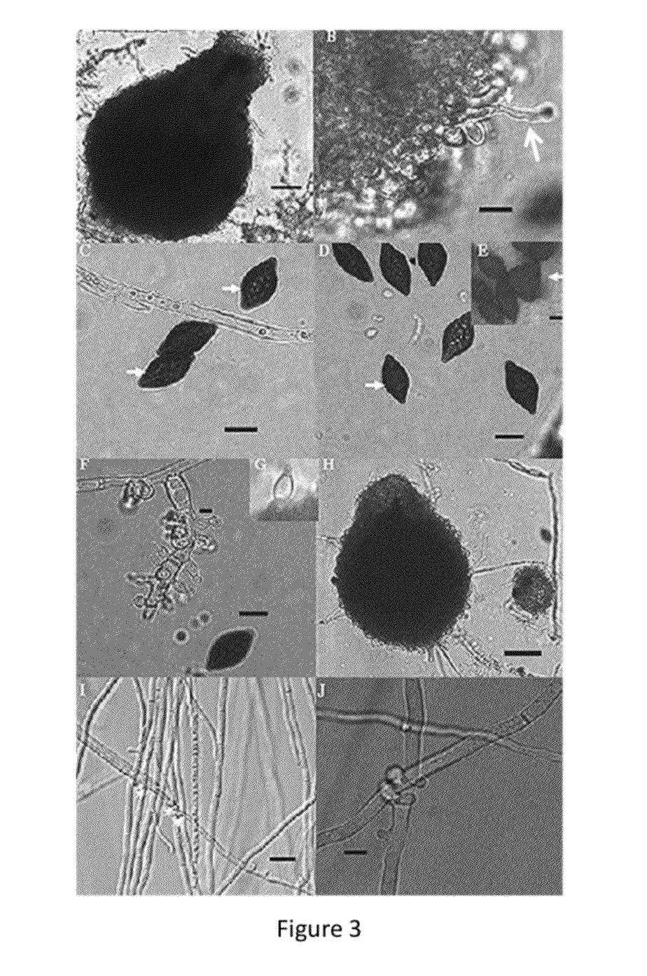

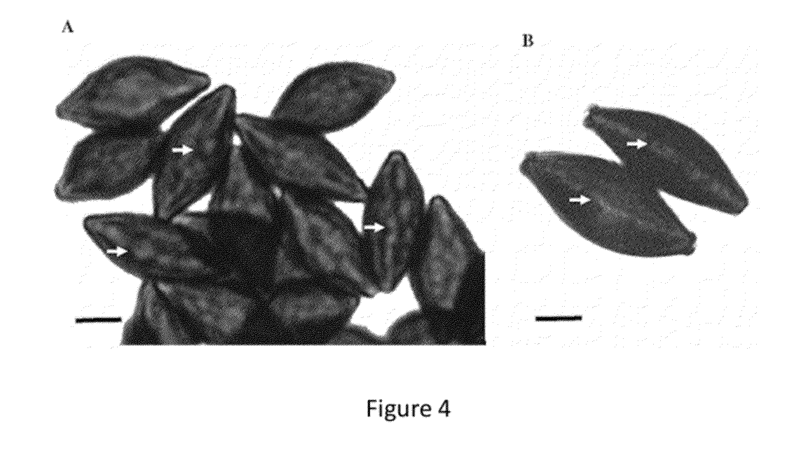

S. mycoparasitica strain IDAC 301008-01 has a unique combination of features shown in FIG. 3. The ascomata height is generally less than 250 μm with a conical to cylindrical neck (FIG. 3A). The setae length is generally less than 40 μm (FIG. 3B) and the spore length is generally less than 23 μm (FIG. 3C). The spores show a conspicuous wall ornamentation and prominent irregular longitudinal ribs indicated by the arrows in FIG. 4A. The spores of Sphaerodes quadrangularis are shown in FIG. 4B for comparison. S. mycoparasitica strain IDAC 301008-01 spores occasionally show a triangular shape (FIGS. 3D and 3E). The formation of starting and mature ascoma of S. mycoparasitica strain IDAC 301008-01 are shown in FIG. 3H. This strain produces simple phialides on the surface of ascoma peridial walls or alternatively, the phialides may be scattered irregularly on ascoma surrounding the hyphae, and on conidiosphores with a distinctive branching pattern (FIGS. 3F and 3G). S. mycoparasitica strain IDAC 301008-01 forms hook-like structures for parasitizing living hyphae of Fusarium (FIGS. 3I and 3J).

### Example 2

Sphaerodes mycoparasitica (SMCD 2220-01) 21° C. isolates were cultured in potato dextrose broth (PDB) culture media. About 3 ml of culture were transferred to 250 ml Ehrlenmeyer flasks containing 50 ml PDB growth medium. The flasks were incubated for 7 days on a rotary shaker (150 rpm) at room temperature.

Extracellular protein extraction: Young mycelia were filtered through Whatman® No. 1 filter paper (Whatman is a registered trademark of Whatman International Ltd., Kent, UK). Filtered culture medium, containing extracellular proteins, and were concentrated by Amicon ultrafiltration centrifuge tube with a 3000 Dalton cut-off membrane by centrifugation at 4000 rpm at 4° C.

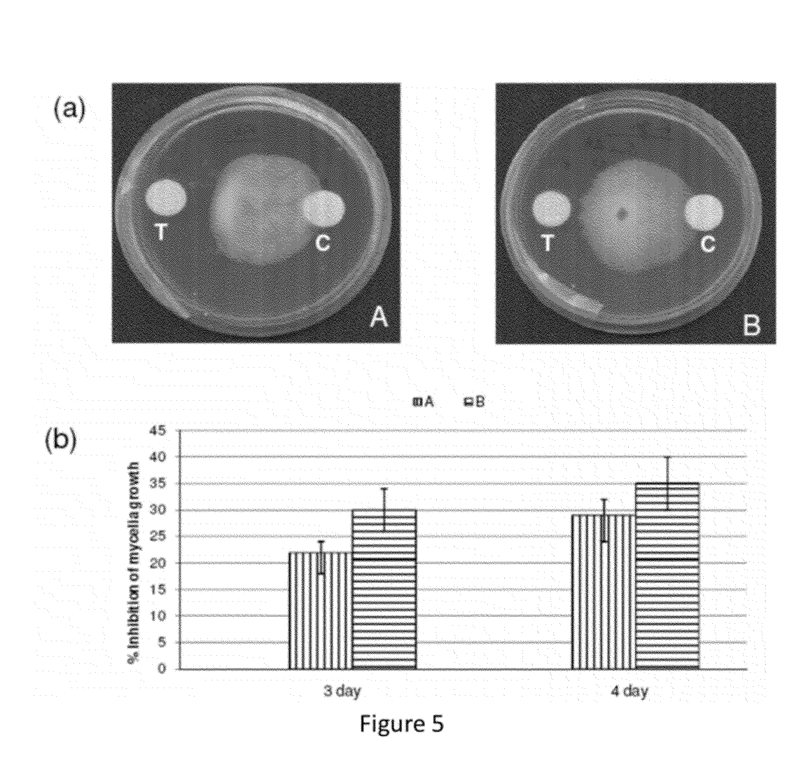

Disc diffusion assay: Antifungal activities of extracellular protein extracts were tested under sterile conditions by radial disc plate diffusion assay as described by Roberts & Selitrennikoff (1986, Biochim. Biophys. Acta, 880: 161-170). The assay of the isolated protein for antifungal activity toward F. oxysporum and F. graminearum was carried out in petri plates containing potato dextrose agar. Mycelial plugs from actively growing fungal plates were placed in the center of the petri plates and sterile filter paper discs (5-mm diameter of Whatman® filter paper no. 1) were placed on the agar surface at a distance of 0.5 cm away from the rim of the mycelial colony. An aliquot (60 μL) containing 2.5 μg of extracellular protein was added to a disk. Sterile distilled water and buffer served as controls. The plates were then incubated at room temperature for 4 days and examined for inhibition. The area of the mycelial colony was measured and the inhibition of fungal growth was determined by calculating the % reduction in area of mycelial colony with the controls (FIG. 5A). After 4^{th} day of germination, about 30% inhibition of the hyphal extension of F. oxysporum and 35% inhibition of hyphal extension of F. graminearum were observed (FIG. 5B)

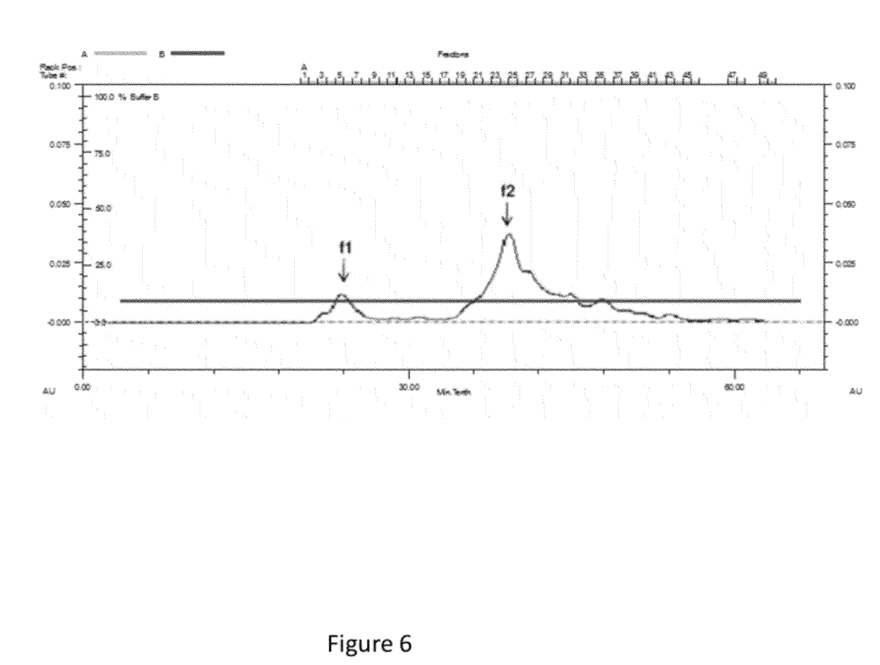

Fast protein liquid chromatography (FPLC) of extracellular proteins: Proteins were fractionated through Superdex 75 GL 10/30 column using FPLC AKTA® purifier system (GE Healthcare, Biosciences AB, CA: AKTA is a registered trademark of GE Healthcare Bio-Sciences AB Ltd., Uppsala, Sweden) according to the manufacturer's instructions. The column was previously equilibrated with sterile water and with 50 mM sodium phosphate buffer, pH 7.0 containing 0.15 M NaCl, followed by protein injection (about 500 μL) and elution of proteins with the same buffer with flow rate of 1.0 ml/min. Fractions of 0.8 ml were collected in each tube. Upon gel filtration on Superdex® 75 (Superdex is a registered trademark of GE Healthcare Bio-Sciences AB Ltd., Uppsala, Sweden), proteins were resolved into two distinct peaks (f1 and f2) and few smaller peaks FIG. 6). Samples from all the peak fractions were pooled, precipitated and tested for their antifungal activity.

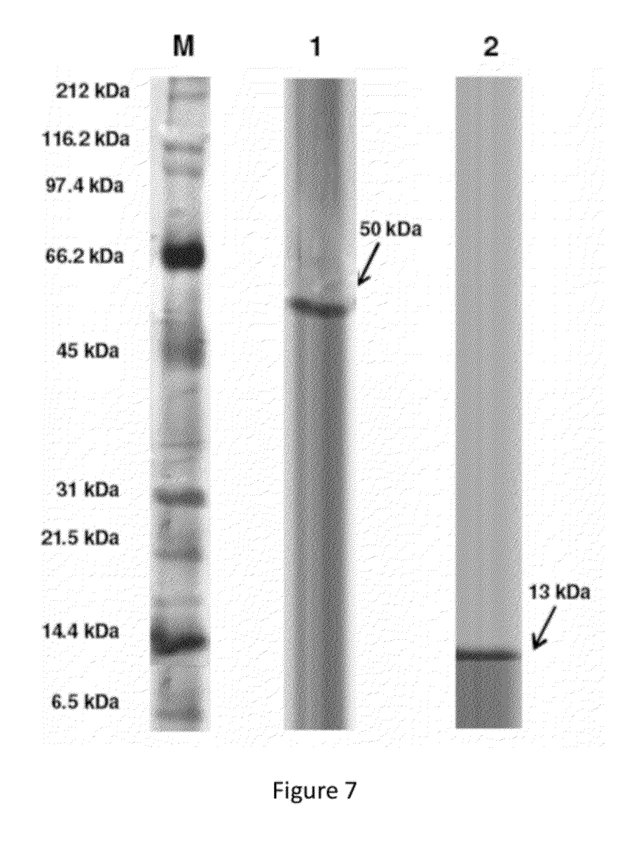

Sodium dodecyl sulfate-polyactylamide gel electrophoresis (SDS-PAGE): SDS-PAGE (12%) of proteins recovered from FPLC fractions was performed according to the method of Laertunli (1970, Nature 227: 680-685). All peaks giving FPLC fractions were pooled and recovered by precipitation in 1:4 volume of chilled acetone and kept at −20° C. overnight. After centrifugation at 12,000 g for 10 min, precipitated proteins (pellets) were dissolved in minimum amount (30 μL) of assay buffer. Proteins were analyzed by SDS-PAGE having 5% stacking gel (pH 6.8) and 12% separating acrylamide gel (pH 8.8) in Tris-glycine buffer (pH 8.3) and appropriate markers. Prior to SDS-electrophoresis, the protein was mixed with an equal volume of sample buffer (60 mM Tris-HCl buffer, 4% SDS, pH 6.8) containing 5% β-mercaptoethanol. A mixture of standard marker proteins (Bio-Rad protein markers, Bio-Rad Laboratories Inc., Mississauga, ON, CA) was used. All samples were heated for 5 min at 95° C. and cooled to room temperature before loading on gel. Proteins were visualized by silver staining method (Bio-Rad Silver staining kit, Bio-Rad Laboratories Inc., Mississauga, ON, CA). Proteins molecular masses were estimated by comparison with the mobilities of standard molecular mass markers. Protein bands of molecular weight 13 kDa and 50 kDa were detected in f1 and f2 peaks respectively (FIG. 7).

Microtitre plate assay: Percentage inhibition of spore germination was performed by microtitre plate assay method (Ghosh, 2006. Ann. Bot. 98: 1145-1153; Yadav et al. 2007, J. Med. Microbiol. 56: 637-644) to test the antifungal activity of proteins recovered from FPLC fractions. The possible toxicity of the fractionated proteins was tested by a percentage growth inhibition assay using the F. oxysporum and F. graminearum. The in vitro antifungal activities of fractionated proteins were determined in 96-well microtiter plates. In microplate wells, 10 μl of potato dextrose broth (PDB; BD Biosciences, Mississauga, ON, CA) was mixed with 3 μl of spore suspensions of F. oxysporum and F. graminearum. An aliquot of 7 μL of different peak containing protein fractions were added to suspensions in microtitre plates (12-8 wells). Water and buffer were used as negative controls. The microtitre plate was then incubated at room temperature in dark. Observations were made for inhibition in spore germination in both untreated and treated wells after 24 h using inverted and fluorescent microscope. The number of germinated and non-germinated spores and percentage of area covered by mycelia in microscope were used to determine the percentage of growth inhibition. The f1 and f2 protein-containing peaks had inhibitory effects on spore germination after 24 hrs compared to the control treatment.

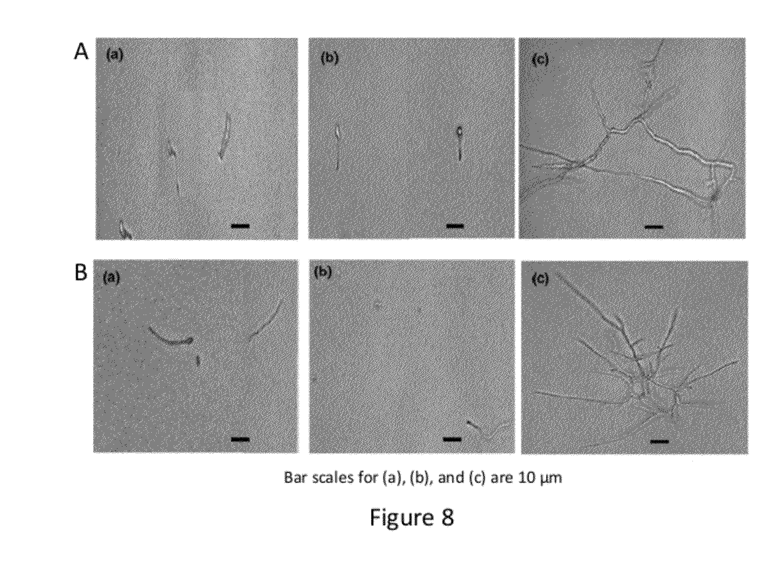

The discharge of spores from sporangia was inhibited by these protein fractions. Secondary branching (mycelia formation) was observed in control treated spores of F. oxysporum (FIG. 8A-c) and F. graminearum (FIG. 8B-c). No branching was observed in f1 and f2 protein-treated spores of F. oxysporum (FIG. 8A-a-b) and F. graminearum (FIG. 8B-a-b). The size of germ tubes (hyphae) from control-treated spores was more than 100 μm compared to 10-15 μm for protein-treated spores (FIGS. 8A-a-b; 8B-a-b).

### Example 3

Active growing mycelia of S. mycoparasitica (SMCD 2220-01) were cultivated on Potato Dextrose Broth (PDB) in a shaking flask at 21° C. for 3 days and then washed. Wet mycelium (0.1 g) were resuspended in 1 ml of PDA medium supplemented 100 μg/mL 3-ADON (Sigma-Aldrich Canada Ltd., Oakville, ON, CA) and incubated for 10 days.

A 0.2 cm² plug S. mycoparasitica was incubated in 1 ml PDB containing 3-ADON at a concentration of 100 μg at RT for 7 days. The sample was analysed for DON degradation by TLC and HPLC assays.

### Extraction of 3-ADON:

3-ADON was extracted by the method disclosed by Vasavada and Hsieh (1987, Appl. Micro. Biotechnol. 26: 517-521). The spent medium in sampled flasks was filtered through the Whatman® filter paper avoiding the mycelia. 3-ADON was extracted from the medium by three 10 ml volumes of ethyl acetate. The mixture of the medium and solvent was vigorously shaken and allowed to stand for 5 min for separation of phases. The organic phase was siphoned off and passed through sodium sulfate to remove residual water. The solvent was allowed to evaporate at room temperature. The residue was redissolved in acetonitrile for analysis.

### Analysis of DON by TLC:

Thin layer chromatography (TLC) was used to analyze the DON. TLC was performed by the method disclosed by Andrea et. al. (2004, J. Basic Microbiol. 44: 147-156). Dried residues were dissolved in 15 μl ethyl acetate and loaded onto silica gel TLC plates coated with fluorescent indicator (60 F₂₅₄, 0.2 mm layer; Merck Frosst Canada Ltd, Kirkland, PQ, CA). The spots were focused in the solvent system with ethyl acetate-toluene (3:1) for 40 min. The gel plates were sprayed with 20% aluminum chloride in 95% ethanol. Trichothecenes were visualized as dark spots under short wavelength UV light (254 nm). Chromatograms were photographed using gel documentation system. A known standard control of pure 3-ADON (Sigma-Aldrich Canada Ltd., Oakville, ON, CA) was employed to compare with other treatments. The sample containing S. mycoparasitica showed significantly less 3-ADON than the control sample.

### High Performance Liquid Chromatography (HPLC)

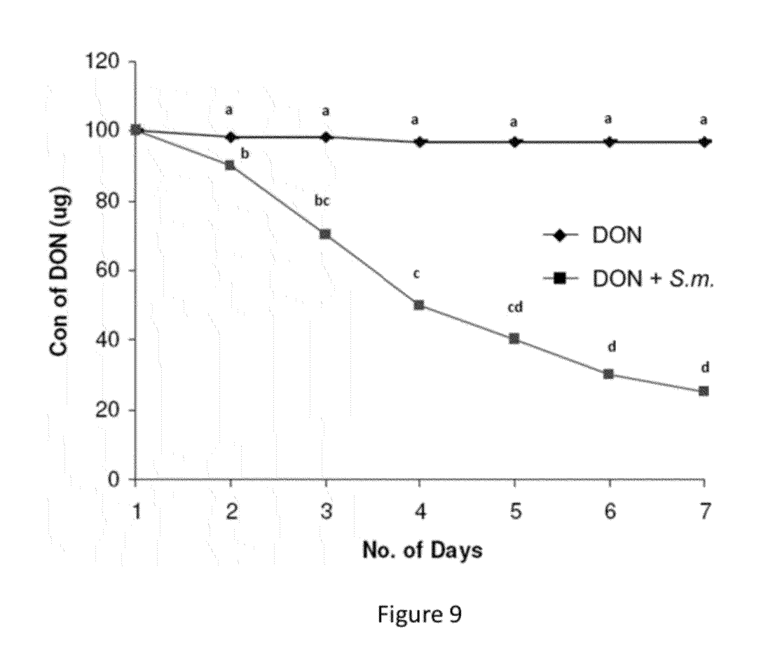

A Water's HPLC with: 250×4.60 mm id Prodigy 5μ ODS (3) 100A, 5μ C₁₈ column (Phenomenex Inc., Torrance, Calif. USA) and a photodiode-array (PDA) detector was used with a gradient solvent system (water-acetonitrile containing 0.005% (v/v) trifluoracetic acid). The PDA detector measured the UV spectrum (200-600 nm). Samples were dissolved in acetonitrile and 20 μl was loaded onto the column using an automatic injector. The DON was eluted with solvent as a mobile phase at a rate of 1 ml min⁻¹. 3-ADON obtained from Sigma-Aldrich (Sigma-Aldrich Canada Ltd., Oakville, ON, CA) was used as a standard. The results are shown in FIG. 9. The sample with S. mycoparasitica contained a much reduced level of 3-ADON.

### Example 4

Potato dextrose agar (PDA), Potato dextrose broth (PDB), Yeast extract, Malt extract agar (MEA), Agar, and Peptone were purchased from BD Biosciences (Mississauga, ON, CA). Streptomycin sulphate, Neomycin sulphate, and other reagents of analytic grade were from Sigma-Aldrich Canada Ltd. (Oakville, ON, CA).

### Fungal Strains and Growth Conditions

Four phytopathogenic Fusarium strains (F. avenaceum, F. sporotrichioides, F. oxysporum, and F. proliferatum); three beneficial fungal inoculants (Trichoderma harzianum (RootShield®), Penicillium bilaii (JumpStart®), and Chaetomium globosum); and one mycoparasitic fungal strain (Sphaerodes mycoparasitica SMCD 2220-01) isolated from F. oxysporum host, were maintained on PDA amended with antibiotics (100 mg/L streptomycin sulphate and 12 mg/L neomycin sulphate) and used throughout this study. S. mycoparasitica mycoparasitic fungal isolate was separated from its F. oxysporum host, and monosporium cultures were achieved according to methods described by Harveson R M, Kimbrough J W, (2001, Int. J. Plant Sci. 162: 403-410) with few modifications. Mature perithecia were picked up, suspended, and shaken in 2-ml sterilized water blanks. The spores-suspension was then spread on PDA plates. After a few minutes, with the assistance of a Carl Zeiss Stemi 1000 dissecting microscope, individual spores were removed and transferred to PDA supplemented with 100 mL/L of Fusarium filtrate. All fungal isolates were maintained in the culture collection of the Saskatchewan Microbial Collection and Database, Canada (SMCD).

### Spores Production

Mycelium plugs from the margin of the active growing monosporial Sphaerodes culture were cut and placed on Modified Leonian's Agar (MLA: Maltose, 6.25 g; Malt extract, 6.25 g; KH2PO4, 1.25 g; Yeast extract, 1.0 g; MgSO₄.7H₂O, 0.625 g; Peptone, 0.625 g; Agar, 20 g, and 1 L of dH₂O). Inoculated MLA plates were incubated at room temperature (23° C.) in dark condition for a month before collecting the spores for germination assays. With the assistance of a dissecting microscope, mature spores exuded from the ascomata and located on the ostiolar opening were harvested by picking up carefully with sterile needles and added to 2 ml tubes in 1 ml sterilized distilled water. The suspension was then filtered through four thin layers of cheesecloth to remove the remaining vegetative cells or mycelia. The density of the spore suspension was counted with a haemocytometer and adjusted to approximately 5-6×10⁵ spores per ml in sterilized water. Freshly prepared aqueous spore suspensions were used.

### Preparation of the Fungal Filtrates

Four pathogenic Fusarium strains and three beneficial fungal strains were grown in shake cultures for 7 days at room temperature (23° C.) in 500 ml flasks, each with 100 ml PDB medium.

After incubation of 7 days in PDB medium, mycelia were removed by filtering through filter paper and the filtrates were then filter-sterilized. Freshly prepared fungal filtrates were employed for the spore germination tests.

Sphaerodes mycoparasitica strain SMCD 2220-01 was found to sporulate on or when inoculated together with Fusarium species, such as F. avenaceum and F. oxysporum. S. mycoparasitica was observed to produce approximately the same amount of ascomata on both F. oxysporum and F. avenaceum. S. mycoparasitica was not found to produce fruiting bodies on other Fusarium or fungal strains such as F. proliferatum, F. sporotrichioides, P. bilaai, T. harzianum, and C. globosum. In the contact zone between these biotrophic mycoparasites and Fusarium species, hook-shaped contact structures were formed.

### Effects of Heat and Cold Treatments on Sphaerodes Spore Germination

Aqueous ascospore suspensions were heat-shocked at 60° C. and 65° C. for 20 min, and cold-treated at 4° C., −20° C., and −70° C. for 5 min and 20 min. The heat- and cold-shocked spores were then transferred and inoculated onto WA and PDA for 1 day and 3 days. The readings of spore germination were checked daily. Spores not subjected to heat and cold treatments were used as control.

There was no germination observed in any of the heat and cold treatment groups for 3 days. Additional observations were continued up to 7 days and no spore germination was observed.

### Germination on Various Media

Aqueous spore suspensions were transferred and inoculated onto the surface of the following media:
1.5% water agar (WA), PDA, MLA, MEA, Carnation leaves agar (CLA);
1.5% water agar plus 100 ml/l of Fusarium filtrate; PDA with 100 ml/l of Fusarium filtrate.

Fusarium strains utilized were F. avenaceum, F. oxysporum, F. proliferatum, and F. sporotrichioides. Ascospore-inoculated plates were then incubated at room temperature (23° C.) for 3 days. Spore germination was examined daily.

The results are summarized in Table 1. Each incubation day for Sphaerodes was analyzed separately. Numbers in each column represent the mean of ascospore germination (in %)±standard deviation. Means within each column for each medium treatment followed by the same letter in superscript are not significantly different at P≦0.05 after Mann-Whitney U test.

TABLE 1
Sporulation of S. mycoparasitica on various media

| | S. mycoparasitica | |
|---|---|---|
| Medium | Day 1 | Day 3 |
| Water agar (WA) | 0 ^{d} | 0 ^{d} |
| F. avenaceum-WA | 11.1 ± 1.7 ^{b} | 35.2 ± 2.6 ^{b} |
| F. oxysporum-WA | 1.6 ± 1.3 ^{c} | 18 ± 1.4 ^{c} |
| F. proliferatum-WA | 0 ^{d} | 0 ^{d} |
| F. sporotrichioides-WA | 0 ^{d} | 0 ^{d} |
| Potato dextrose agar (PDA) | 0 ^{d} | 0 ^{d} |
| F. avenaceum-PDA | 21.5 ± 1.4 ^{a} | 63.2 ± 2.4 ^{a} |
| F. oxysporum-PDA | 2.5 ± 2 ^{c} | 37.5 ± 2 ^{b} |
| F. proliferatum-PDA | 0 ^{d} | 0 ^{d} |
| F. sporotrichioides-PDA | 0 ^{d} | 0 ^{d} |

### Effects of Fungal Filtrates on Spore Germination

The aqueous spore suspensions were suspended in the filtrates of four separate pathogenic Fusarium and three beneficial fungal strains for 1 day and 3 days in the ratio of 1:2 (1 part of aqueous spore suspension: 2 parts of fungal filtrate), and the filtrate-suspended spores were then transferred and inoculated onto PDA for an additional 1 day. Control treatments were suspended with sterilized distilled water or PDB.

### Spore Germination

Microscopic assessments of ascospore germination were conducted after incubation for 1 day and 3 days. Percentage of germinated spores was obtained by scoring the spores on the Petri dish through utilizing the 200× and 400× objectives of the Carl Zeiss Axioskop 2 microscope and systematically choosing 50 spores, starting at the top right corner and continuing to count until 50. Each drop of ascospore suspensions on a medium plate was considered as a subunit, and there were three subunits per plate. Each medium plate for each treatment was replicated, there were three replicas per treatments. The experiments were repeated twice. An ascospore was only considered as germinated spore when the germ tube was visibly noticeable. Germinated ascospores were counted and recorded as a percentage of the total ascospore number.

The results are summarized in Table 2. Numbers in each column represented mean of ascospore germination (in %) ±standard deviation. Each incubation day for Sphaerodes was analyzed separately. Means within each column of Sphaerodes for each filtrate-suspension treatment followed by the same letter in superscript are not significantly different at P≦0.05 after Mann-Whitney U test.

TABLE 2
Effects of fungal filtrates on S. mycoparasitica spore germination

| | Spore germination (in %) | | | |
|---|---|---|---|---|
| Shpaerodes sp. | 1 d | 1 d sus + | 3 d | 3 d sus + |
| Treatments | suspension | 1 d PDA | suspension | 1 d PDA |
| Water | 0 ^{c} | 0 ^{e} | 0 ^{e} | 2 ± 1.2 ^{c} |
| PDB | 0 ^{c} | 0 ^{e} | 0 ^{e} | 2.8 ± 1.9 ^{c} |
| F. avenaceum- | 89.2 ± 6.2 ^{a} | 91 ± 8.4 ^{a} | 93.2 ± 6.7 ^{a} | 94.2 ± 7.9 ^{a} |
| filtrate | | | | |
| F. oxysporum- | 0 ^{c} | 81.8 ± 11 ^{b} | 85 ± 7.9 ^{b} | 91 ± 10 ^{a} |
| filtrate | | | | |
| F. proliferatum- | 2.6 ± 3.6 ^{b} | 3.2 ± 4.6 ^{d} | 10 ± 7.4 ^{c} | 11.2 ± 8 ^{b} |
| filtrate | | | | |
| F. sporotrichioides- | 0 ^{c} | 3 ± 2.8 ^{d} | 3.4 ± 3.3 ^{d} | 10 ± 5.6 ^{b} |
| filtrate | | | | |
| P. bilaii-filtrate | 0 ^{c} | 0 ^{e} | 0 ^{e} | 0 ^{c} |
| T harzianum-filtrate | 0 ^{c} | 9 ± 1.1 ^{c} | 1.6 ± 1.6 ^{d} | 10.4 ± 1.5 ^{b} |
| C. globosum-filtrate | 0 ^{c} | 0 ^{e} | 0 ^{e} | 0 ^{c} |

Values followed by same letters in superscript within each column are not significantly different using Tukey's range test (P < 0.05).

### Example 5

Since F. graminearum 3-ADON is the most pathogenic and mycotoxigenic in wheat, 3-ADON was used to quantify mycoparasite-Fusarium-wheat root interactions under Phytotron controlled conditions.

### Fungal Strains and Growth

Fusarium strains: F. graminearum 3-ADON strain SMCD2243, biotrophic mycoparasite Sphaerodes mycoparasitica SMCD2220-01, and Trichoderma harzianum T-22 (RootShield—commercial product) as a control strain with mycoparasitic properties were maintained on PDA amended with antibiotics.

### Taxon Specific Primers

Specific primer sets for: F. graminearum-Fgl 2NFiR (Nicholson et al. 1998. Plant Pathology 53: 17-37.); Trichoderma-TGP4-F IR (Kim and Knudsen 2008. Applied Soil Ecology 40: 100-108); and S. mycoparasitica SMCD2220-01-SmyITSF/R were used in this study.

### Mycoparasite/Fusarium/Plant Interactions

Wheat CDC-TEAL 2001 plants were inoculated with mycotoxigenic and pathogenic Fusarium as well as Trichoderma (control mycoparasite) or Sphaerodes SMCD2220-01, and were subjected to RT-PCR quantification to assess the amount of fungal DNAs in roots of wheat under controlled conditions after 14 days of incubation in Phytotron conditions.

### Growth Conditions and Fungal Inoculation

Five mycelial plugs from S. mycoparasitica, T. harzianum, and F. graminearum were cut, transferred and grown in three separate shake cultures for 14 d at room temperature (23° C.) in 500 mL flasks each with 100 mL of PDB (potato dextrose broth). After incubation of 14 d in PDB medium, the fungal cultures were filtered through a Whatman® No. 1 filter paper to remove the liquid medium. The mycelia were then transferred to 50 mL sterile Falcon tubes with 20 sterile glass beads and 40 mL of autoclaved distillated water. The Falcon tubes filled with mycelia materials were then vortexed vigorously for 1 min to separate the mycelia into smaller pieces. Mycelial suspension was filtered through 2 layers of cheesecloth to remove the glass beads and bigger mycelial clumps. The flow-through was then used as mycelial suspension stock) (10°) for serial dilution. Stock of mycelial suspension was further diluted into a series spanning from 10⁻², 10⁻³, and 10⁻⁴. This dilution series was plated on PDA using the pour plate method. The number of CFU (colony forming units) was counted and recorded. Mycelial suspensions were adjusted with sterile water to about 10⁻⁵-10⁻⁶ CFU/mL for S. mycoparasitica and T. harzianum, and to about 10⁻⁴-10⁻⁵ CFU/mL for F. graminearum.

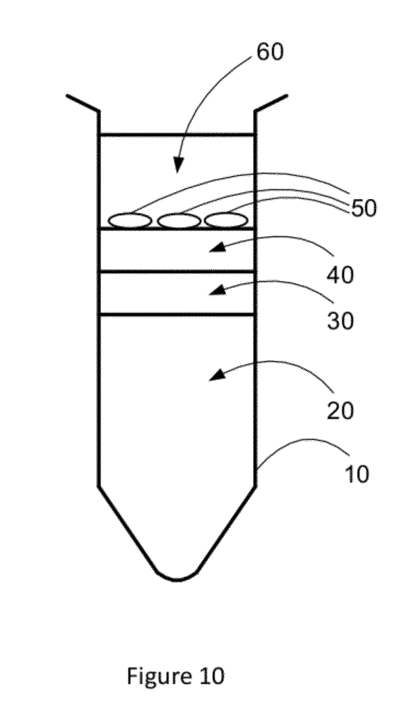

Quantification of interactions between mycoparasite-pathogen-wheat roots was conducted on the spring wheat cultivar CDC-TEAL 2001. Wheat plants were grown in containers (4×4×16 cm) with 10 g of different layers of soil-less growing mix (FIG. 10). All the seeds were surface-sterilized prior to sowing. The containers 10 were lined with filter paper and packed with 6 g of Pro-Mix® soil-less mix (Sun Gro Horticulture Canada Ltd., Delta, BC, CA; Pro-Mix is a registered trademark of Premier Horticulture Ltd., Riviere-du-Loop, PQ, CA) which comprised the first layer 20. This layer 20 was then overlayed with a second layer 30 (1 g) of either Pro-Mix® mix amended and homogenized with ˜5-6×10⁴ CFU of F. graminearum mycelial suspension or alternatively with Pro-Mix® mix with water only. The second layer 30 was followed by a third layer 40 (1 g) of either Sunshine® peat moss (Sunshine is a registered trademark of Sun Gro Horticulture Canada Ltd., Seba Beach, AB, CA) supplemented and homogenized with ˜5-6×10⁵ CFU of S. mycoparasitica or alternatively with T. harzianum mycelial suspension or alternatively with water only. Six spring wheat seeds 50 were then sowed on top of the third layer 40 and topped with a fifth layer 60 of 2 g of Sunshine® peat moss (FIG. 10). Spring wheat seeds 50 were germinated and grown under a 16-h photoperiod (22° C. day/15° C. night) with light intensity of 250 μmol m⁻² s⁻¹, watered every 2 d, and fertilized every 14 d using 1300 ppm of NPK (20-20-20) fertilizer. All treatments in the experiment were in three replicates and the experiment was repeated twice.

At the mid-seedling growth, corresponding to Zadok's growth stage 13 (Zadoks et al. 1974, Weed Research 14:415-421), wheat plants with their roots were removed from the pots and washed under running tap water to remove all the soil particles. Washed roots were dried with filter papers. Number of germinated seeds, total biomass, root biomass, total length, and root length were counted and measured. Percentage of seed germination was calculated with the following formula: (Number of germinated seeds in particular treatment/number of germinated seeds in control treatment)×100%. The roots were then subjected to total DNA extraction with DNeasy® Plant Mini Kit (Qiagen Inc., Mississauga, ON, CA). Extracted total DNAs from roots of different treatments were employed in real-time PCR quantification.

### Statistical Analyses

Root biomass (g), total biomass (g), root length (cm), total length (cm), seed germination (%); and S. mycoparasitica, T. harzianum, and F. graminearum genomic DNA quantification from the roots of spring wheat plants were analyzed by using analysis of variance (ANOVA). Log10 transformations were carried out whenever required to meet the ANOVA requirements. Multiple comparisons for more than two samples were analyzed by utilizing Tukey's studentized range test at P=0.05 (SPSS 1990).

### Wheat Growth and Fungal Inoculation

Root biomass, total biomass, root length, total length, and seed germination of F. graminearum infected spring wheat were significantly increased with the treatments of S. mycoparasitica compared to inoculation with F. graminearum alone (Table 3).

TABLE 3
Effects of SMCD2220-01 (SM) and F. graminearum 3-ADON (Fgra) inoculation
treatments on root biomass (g), total biomass (g), root length (cm), total length (cm)
and seed germination (%) of spring wheat plants

| | Root biomass | Total biomass | Root length | Total length | Seed |
|---|---|---|---|---|---|
| Treatment | (g) | (g) | (cm) | (cm) | germination (%) |
| Control | 0.27 ± 0.06 ^{b} | 0.56 ± 0.56 ^{b} | 9.63 ± 0.74 ^{b} | 31.88 ± 2.39 ^{c} | NA* |
| SM | 0.32 ± 0.03 ^{ab} | 0.69 ± 0.69 ^{a} | 13.88 ± 1.11 ^{a} | 38.13 ± 2.76 ^{ab} | 101 ± 2 ^{a} |
| Fgra | 0.18 ± 0.03 ^{c} | 0.38 ± 0.48 ^{c} | 7.88 ± 1.89 ^{c} | 23.75 ± 1.98 ^{d} | 31.25 ± 1.4 ^{c} |
| SM-Fgra | 0.27 ± 0.03 ^{b} | 0.54 ± 0.54 ^{b} | 12.13 ± 1.35 ^{ab} | 32.13 ± 3.91 ^{be} | 87.25 ± 2 ^{b} |

*Values expressed are means of six replicates ± standard deviation of the mean. Values followed by same letters in superscript within each column are not significantly different using Tukey's range test (P < 0.05).

Table 3, the mycoparasite S. mycoparasitica demonstrates both biomass stimulation and bioprotection or biocontrol.

### Conformation of the Sphaerodes-Specific Primer Set

The SmyITSF/R primer set was tested with S. mycoparasitica, five Fusarium species, two different ascomycetous fungal isolates two zygomycete fungi, and three basidiomycetous fungal strains. This primer set only amplified S. mycoparasitica.

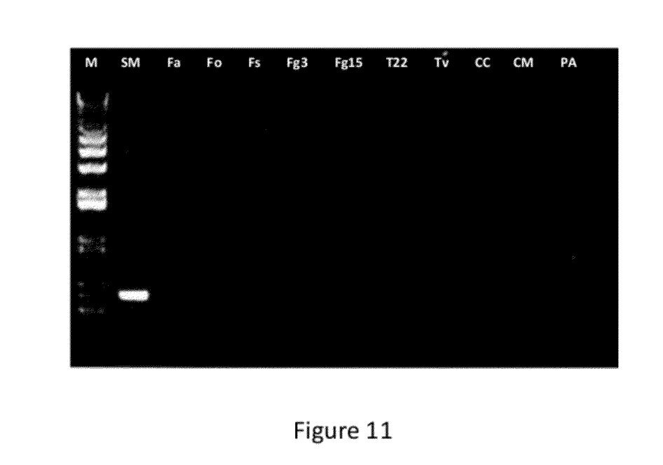

FIG. 11 shows SmyITSF/R primers amplified PCR products for S. mycoparasitica (SM), five Fusarium strains (Fa=F. avenaceum, Fo=F. oxysporum, Fs=F. sporotrichioides, Fg3=F. graminearum chemotype 3, and Fg15=F. graminearum chemotype 15), two Trichoderma species (T22=T. harzianum T22 and Tv=T. viride), two Cladosporium species (CC=C. cladosporioides and CM=C. minourae), and Penicillium aurantiogriseum (PA) were electrophoresed on 1% agarose gel at 100 V for 20 minutes. The size of the band is around 300 to 400 bp.

### Standard Curves

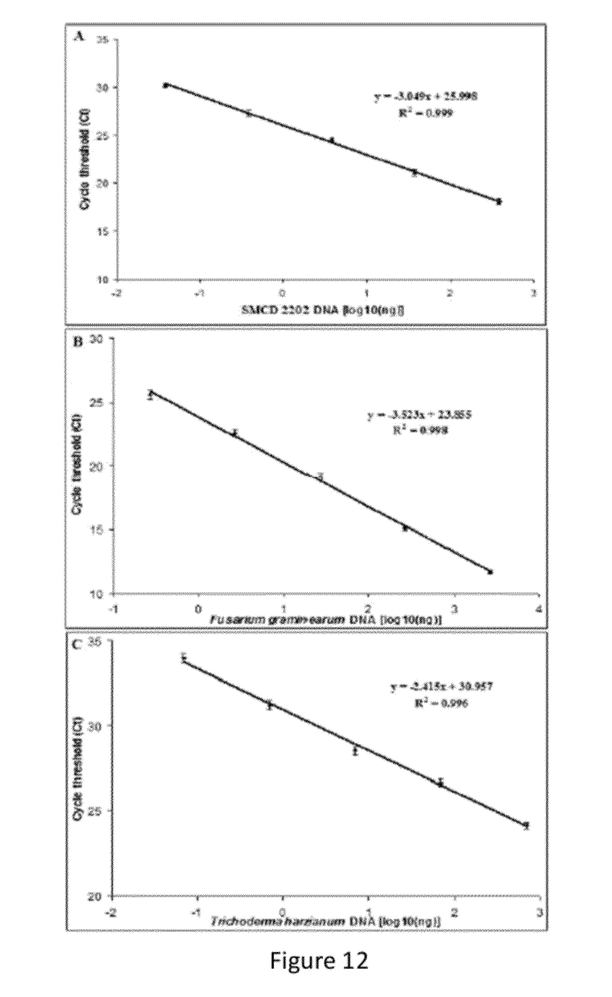

The standard curves based on known diluted concentrations of DNAs from S. mycoparasitica, F. graminearum, and T. harzianum were constructed. Standard curves were achieved using a series of 10-fold diluted DNA spanning from 3.8×10² to 3.8×10⁻² ng for S. mycoparasitica, 2.7×10³ to 2.7×10⁻¹ ng for F. graminearum chemotype 3, and 7.0×10² to 7.0×10⁻² ng for T. harzianum. Quantification demonstrated linear relation (r²=0.999 for S. mycoparasitica, r²=0.998 for F. graminearum, and r²=0.996 for T. harzianum) between log₁₀ of fungal genomic DNA (in ng/μl) and real-time PCR threshold cycles (Ct) (threshold fluorescence signal of 0.025 was used for all three fungal isolates) (FIGS. 12A, 12B, and 12C).

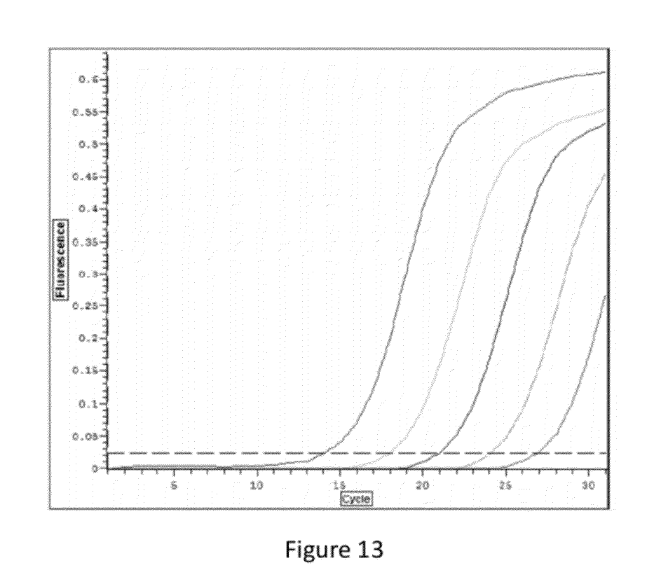

FIG. 13 shows RT-PCR sigmoidal coloured curves for Sphaerodes mycoparasitica (SMCD 2220-01), with 0.025 fluorescence line, showing the range of 3.8×10² to 3.8×10⁻² ng in a ten-fold decreasing manner.

### RT-PCR Confirmation of the Sphaerodes-Biocontrol Effects

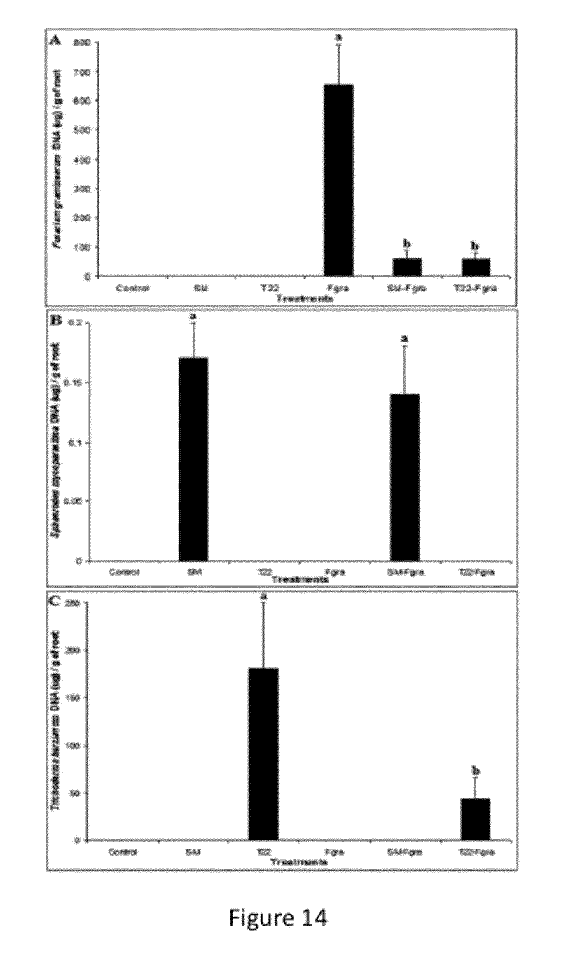

Real-time PCR, which evaluated quantity of S. mycoparasitica SMCD2220-01 and F. graminearum, confirmed that amounts of F. graminearum DNA detected in treatments with S. mycoparasitica SMCD2220-01 and T. harzianum were significantly reduced (FIG. 14A). Previously, treatments with mycoparasitic Sphaerodes retispora had been observed to show significant suppression of F. oxysporum in watermelon plants (Harveson et al. 2002, Plant Dis. 86: 1025-1030). The amount of S. mycoparasitica DNA detected was not significantly different between wheat inoculated with F. graminearum or without Fusarium (FIG. 14B). The amount of T. harzianum DNA detected in the treatment inoculated with F. graminearum was observed to be significantly reduced, as compared to non-Fusarium treatment (FIG. 14C).

In conclusion, during in vitro culture-based studies, only S. mycoparasitica SMCD2220-01 was observed to enhance wheat seed germination and formation of secondary roots, whereas T-22 induced post-emergence damping-off symptoms. Under controlled conditions in a phytotron, S. mycoparasitica SMCD2220-01 was able to reduce the quantity of F. graminearum in spring wheat root, as well as improving the survival and growth of the spring wheat seedlings. In contrast to T. harzianum, the amount of S. mycoparasitica SMCD2220-01 DNA detected was not significantly different between wheat inoculated with F. graminearum and without Fusarium. Hence, S. mycoparasitica SMCD2220-01 could be a better biocontrol candidate for the F. graminearum pathogen in wheat.

### Example 6

Quantitative RT-PCR results from testing Mycoparasite-Fusarium interaction on wheat host using growing conditions disclosed herein also confirmed the efficiency of S. mycoparasitica SMCD2220-01 for significantly decreasing an accumulation of genes implicated in Fusarium DON-mycotoxin production.

Strain used: F. graminearum 3-ADON SMCD2243, F. graminearum 15-ADON SMCD2244 and S. mycoparasitica SMCD2220-01 strains.

Primer sets used: Tox5-1/2 for Fusarium (Wu et al. 2002. J. Environ Monit. 4:377-382) and SmyITSF/R (disclosed herein) for S. mycoparasitica.

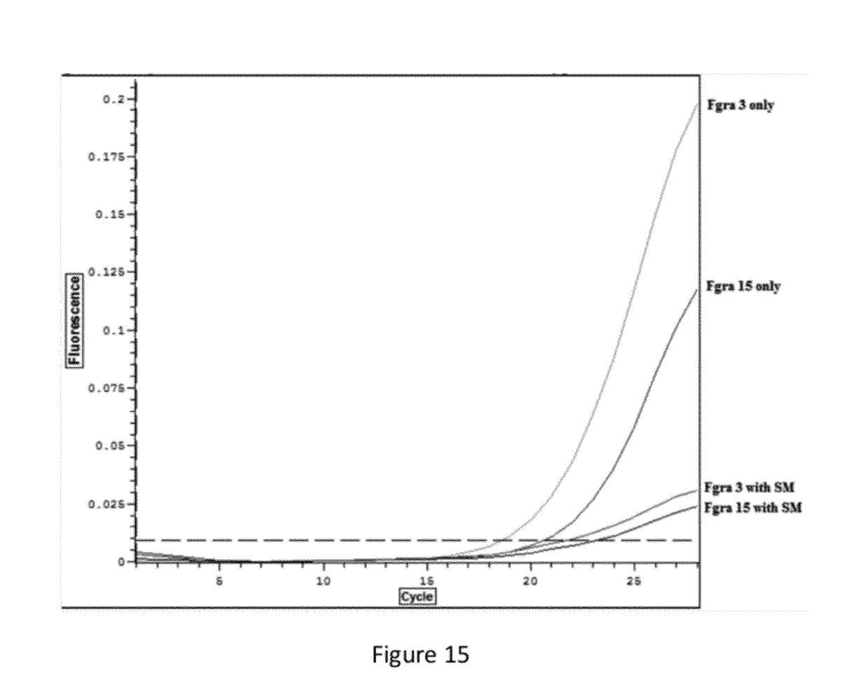

Results are summarized in FIG. 15. Real-time fluorescence curves of tri5 gene sequences amplified by using Tox5-1/2 primer set from total DNA extracted from dual-culture assays of Fusarium graminearum strains and pre-inoculated Sphaerodes mycoparasitica SMCD2220-01 (SM) or singly grown F. graminearum 3-ADON and 15-ADON chemotypes.

### Example 7

The effects of filtrates collected from different Fusarium sp. on the germination of Sphaerodes ascospores were assessed in this study. Also assessed were the ascospore germination patterns.

Media, Reagents, and chemicals: Potato dextrose agar (PDA), potato dextrose broth (PDB), yeast extract, malt extract agar (MEA), agar, and peptone were purchased from Difco (Becton Dickinson Diagnostics, Sparks, Md.). Streptomycin sulphate, Neomycin sulphate, and other reagents of analytic grade were from Sigma-Aldrich (Oakville, ON, CA).
Fungal strains and growth conditions: Four phytopathogenic Fusarium strains (F. avenaceum SMCD 2241, F. oxysporum SMCD 2242, F. proliferatum (Matsush.) Nirenberg SMCD 2244, and F. sporotrichioides Sherb SMCD 2243), three beneficial fungal inoculants: T. harzianum (RootShield®), P. bilaii ([JumpStart®), and Chaetomium globosum Kunze; and one mycoparasitic fungal strain, S. mycoparasitica were maintained on PDA amended with antibiotics (100 mg/L streptomycin sulphate and 12 mg/L neomycin sulphate) and used throughout this study. An isolate of the mycoparasitic fungus, S. mycoparasitica, was separated from its F. oxysporum host on myclobutanil agar (MBA) selective medium as previously described and a monosporic culture was achieved by picking up mature perithecia which were then suspended and shaken in 2-mL sterile distilled water blanks to encourage release of ascospores. The ascospore-suspension was then spread on PDA plates. After a few minutes, a Carl Zeiss Stemi 1000 dissecting microscope was used to identify individual ascospores, which were removed and transferred to PDA supplemented with 100 mL/L of Fusarium filtrate (F. avenaceum and F. oxysporum; 1:1). All fungal isolates were maintained in the culture collection of the Saskatchewan Microbial Collection and Database, Canada (SMCD).
Ascospore production: Mycelium plugs from the margin of the actively growing monosporic-derived S. mycoparasitica culture were cut and placed on Modified Leonian's Agar (MLA: maltose, 6.25 g; malt extract, 6.25 g; KH₂PO₄, 1.25 g; yeast extract, 1.0 g; MgSO₄.7H₂O, 0.625 g; peptone, 0.625 g; agar, 20 g, and 1 L of dH₂O) (Malloch and Cain 1971, Canadian Journal of Botany 49: 839-846). Inoculated Modified Leonian's Agar (MLA) plates were incubated at room temperature (23° C.) under darkness conditions for a month before collecting spores for germination assays. Mature ascospores were collected according to Goh and Vujanovic 2010, Botany 88: 1033-1043.
Preparation of the fungal filtrates: Four pathogenic Fusarium strains and three beneficial fungal strains were grown in shake cultures for 7 d at room temperature (23° C.) (with 250 rpm) in 500 mL flasks, each with 100 mL PDB medium prior to fungal filtrates extraction. After incubation, the mycelia were removed by filtering through filter paper (Whatman® Grade No. 1) and the filtrates were then filter-sterilized with 0.02-μm-pore-size nitrocellulose filter (Fisher Scientific Ltd., Nepean, ON, CA). Only fresh preparations of fungal filtrates were employed for the spore germination test in this study.
Effect of heat and cold treatments on S. mycoparasitica spore germination: Heat activation and cold-shock treatments were performed to investigate ascospore germination in S. mycoparasitica. Aqueous ascospore suspensions were heat-shocked at 60 and 65° C. for 20 min, and cold-treated at 4, −20, and −70° C. for 5 min and 20 min. The heat- and cold-shocked spores (10 μL) were then transferred and inoculated onto water agar (WA) and PDA for 1 d and 3 d. Spore germination was checked daily. Spores not subjected to heat and cold treatments were used as controls. All treatments were in three replicates and experiment was repeated twice.

No germination was observed in all the heat and cold treatments over a 7 d period.

Germination on various media: Aqueous spore suspensions (10 μL) of S. mycoparasitica were transferred and inoculated onto the surface of the following media: 1.5% water agar (WA), PDA, MLA, MEA, Carnation leaf agar (CLA) (Tschanz et al. 1976, Mycologia, 68: 327-340), 1.5% water agar plus 100 mL/L of Fusarium filtrate, and PDA with 100 mL/L of the Fusarium filtrate. Fusarium filtrates were created using F. avenaceum, F. oxysporum, F. proliferatum, and F. sporotrichioides. S. mycoparasitica ascospore-inoculated plates were then incubated at room temperature (23° C.) for 3 d, and spore germination was examined daily. All treatments were in three replicates and experiment was repeated twice.

There was no spore germination on 1.5% WA, PDA, MLA, MEA, and CLA. The effects of 1.5% WA and PDA media amended with 100 mL/L of Fusarium-filtrates on spore germination were examined and are summarized in Table 4.

No germination was recorded on day 3 initially and observations were continued up to 7 d on WA or PDA alone and WA or PDA with either F. proliferatum or F. sporotrichioides. However, when either F. avenaceum or F. oxysporum filtrates were added to WA and PDA, the percentage of ascospore germination in S. mycoparasitica drastically increased after 3 d incubation. On day 3, germination increased on the PDA amended with Fusarium-filtrate, compared to growth on WA supplemented with Fusarium-filtrate, for S. mycoparasitica.

TABLE 4
Percentage germination of Sphaerodes mycoparasitica ascospore
on various types of Fusarium-filtrate supplemented media, including
water, agar and potato dextrose agar checks

| S. mycoparasitica | Spore germination (%)* | |
|---|---|---|
| Medium | Day 1** | Day 3** |
| Water agar (WA) | 0 ^{d} | 0 ^{d} |
| F. avenaceum-WA | 11.1 ± 1.7 ^{b} | 35.2 ± 2.6 ^{b} |
| F. oxysporum-WA | 1.6 ± 1.3 ^{c} | 18 ± 1.4 ^{c} |
| F. proliferatum-WA | 0 ^{d} | 0 ^{d} |
| F. sporotrichioides-WA | 0 ^{d} | 0 ^{d} |
| Potato dextrose agar (PDA) | 0 ^{d} | 0 ^{d} |
| F. avenaceum-PDA | 21.5 ± 1.4 ^{a} | 63.2 ± 2.4 ^{a} |
| F. oxysporum-PDA | 2.5 ± 2 ^{c} | 37.5 ± 2 ^{b} |
| F. proliferatum-PDA | 0 ^{d} | 0 ^{d} |
| F. sporotrichioides-PDA | 0 ^{d} | 0 ^{d} |

*Numbers in each column represented mean of ascospore germination (in %) ± standard deviation.
**Each incubation day for Sphaerodes was analyzed separately. Means within each column for each medium treatment followed by the same letter in superscript are not significantly different at P ≦ 0.05 after Kruskal-Wallis test.

Effects of fungal filtrates on spore germination: Fungal filtrates from four different phytopathogenic Fusarium species and three beneficial fungal isolates were employed to study spore germination of S. mycoparasitica, and host specificity response. Aqueous spore suspensions of S. mycoparasitica were suspended in the filtrates of four separate pathogenic Fusarium spp. and three beneficial fungal strains for 1 d and 3 d in the ratio of 1:2 (1 part of aqueous spore suspension: 2 parts of fungal filtrate). Filtrate-suspended spores (10 μL) were then transferred and inoculated onto PDA for an additional day (spore germination was counted on PDA plate). Spore germination was count according to Goh and Vujanovic (2010, Botany, 88: 1033-1043) after an additional day of inoculation on PDA medium. Control treatments were suspended with sterilized distilled water or PDB. Abbreviations for different treatments at four separate chronosequences throughout the experiment: 1 d Sus, 1 d Sus+1 d PDA, 3 d Sus, and 3 d Sus+1 d PDA represent 1 day Fusarium-filtrate suspension, 1 day filtrate suspension with an additional day incubation on PDA medium, 3 day suspension, and 3 day filtrate suspension with an additional day incubation on PDA, respectively.

No spore germination was observed in the treatments with P. bilaii and C. globosum fungal filtrates. Both water and PDB suspension controls appeared to trigger approximately 1.8-3.8% spore germination for S. mycoparasitica, on day 3 with an additional day on PDA only (Table 5). Ascospores of S. mycoparasitica suspended in filtrates of pathogenic F. sporotrichioides and beneficial T. harzianum showed no germination on day 1 in suspension, but low number of ascospore germination was observed after 1 day in suspension followed by an additional day on PDA, and in the other incubation treatments. Ascospores suspended in F. proliferatum-filtrate were observed to germinate in low abundance for 1 d, 1 d with an additional day on PDA, 3 d, and 3 d plus an additional day on PDA. Sphaerodes ascospores showed the highest number of germinated ascospores in the F. avenaceum-filtrate suspension. In the day 1 suspension treatments, ascospore germination of S. mycoparasitica in F. avenaceum-filtrate was significantly higher (89.2%) than in other treatments. When ascospores of the S. mycoparasitica were suspended in F. oxysporum-filtrate, the amount of spore germination was increased compared to control. There was no germination recorded for the 1 d suspension in the F. oxysporum-filtrate treatment. However, a low number of ascospores were stimulated in F. oxysporum-filtrate suspension on the second day. Based on the ascospore germination rate and response, S. mycoparasitica showed high specificity to F. avenaceum and F. oxysporum.

TABLE 5
Percentage spore germination of Sphaerodes mycoparasitica ascospores suspended in different
Fusarium and biocontrol fungi filtrates assesed throughout four chronosequences. Control treatments
were suspended with sterilized distilled water (SDW) or potato dextrose broth (PDB).

| S. mycoparasitica | Spore germination (%)* | | | |
|---|---|---|---|---|
| Treatment** | 1 d Suspension | 1 d Sus + 1 d PDA | 3 d Suspension | 3 d Sus + 1 d PDA |
| SDW | 0 ^{c} | 0 ^{e} | 0 ^{e} | 2 ± 1.2 ^{c} |
| PDB | 0 ^{c} | 0 ^{e} | 0 ^{e} | 2.8 ± 1.9 ^{c} |
| F. avenaceum-filtrate | 89.2 ± 6.2 ^{a} | 91 ± 8.4 ^{a} | 93.2 ± 6.7 ^{a} | 94.2 ± 7.9 ^{a} |
| F. oxysporum-filtrate | 0 ^{c} | 81.8 ± 11 ^{b} | 85 ± 7.9 ^{b} | 91 ± 10 ^{a} |
| F. proliferatum-filtrate | 2.6 ± 3.6 ^{b} | 3.2 ± 4.6 ^{d} | 10 ± 7.4 ^{c} | 11.2 ± 8 ^{b} |
| F. sporotrichioides-filtrate | 0 ^{c} | 3 ± 2.8 ^{d} | 3.4 ± 3.3 ^{d} | 10 ± 5.6 ^{b} |
| P. bilaii-filtrate | 0 ^{c} | 0 ^{e} | 0 ^{e} | 0 ^{c} |
| T. harzianum-filtrate | 0 ^{c} | 9 ± 1.1 ^{c} | 1.6 ± 1.6 ^{d} | 10.4 ± 1.5 ^{b} |
| C. globosum-filtrate | 0 ^{c} | 0 ^{e} | 0 ^{e} | 0 ^{c} |

*Numbers in each column represented mean of ascospore germination (in %) ± standard deviation.
**Each incubation day for S. mycoparasitica was analyzed separately. Means within each column of S. mycoparasitica for each filtrate-suspension treatment followed by the same letter (in superscript) are not significantly different at P ≦ 0.05 after Kruskal-Wallis test.

Spore germination assessments: Microscopic assessments of ascospore germination were conducted after incubation for 1 d and 3 d (spore germination was counted on suspensions). Percentage of germinated spores was obtained by scoring the spores on a Petri dish while observing them through the 200× and 400× objectives of the Carl Zeiss Axioskop 2 microscope and systematically choosing 50 spores, starting at the top right corner and continuing to count until 50. Each drop of the ascospore suspension on a growth medium plate was considered as a subunit, and there were three subunits or replicates per plate. The experiments were repeated twice. An ascospore was only considered germinated when the germ tubes exceeded the width of the ascospore (approximately 12 μm). Germinated ascospores were counted and recorded as a percentage of the total ascospore number that was counted.

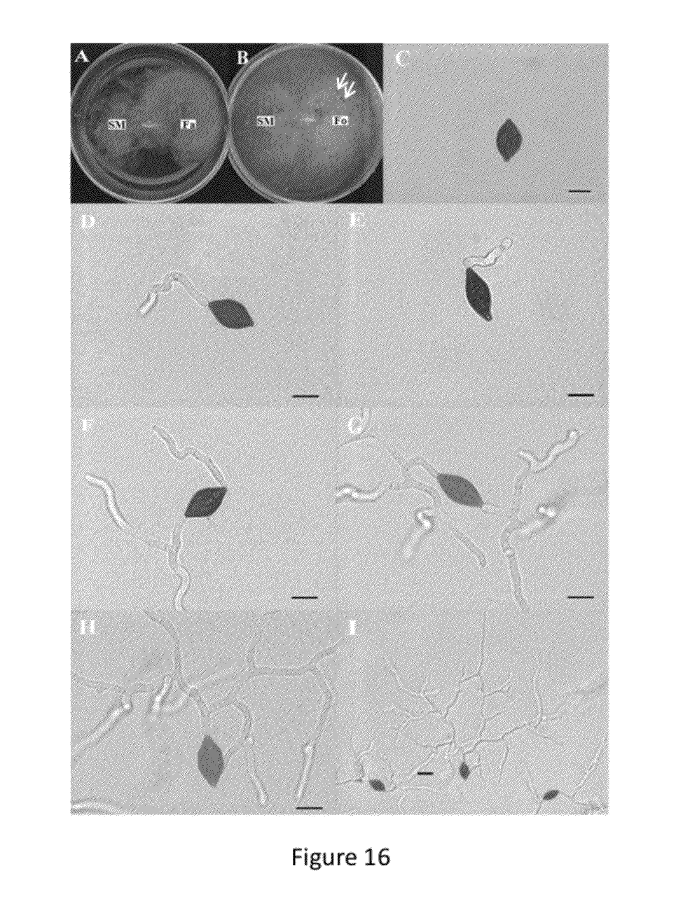

Ascospores from S. mycoparasitica showed two kinds of germination patterns. Single polar germination was more prevalent in the F. oxysporum-filtrate suspension (FIGS. 16A, 16B, 16D, 16E). A small number of spores from S. mycoparasitica found to produce shorter double-polar germination in the treatment with F. oxyporum-filtrate at 3 d suspension with additional 1 d on PDA (FIG. 16F). In the F. avenaceum-filtrate suspension, ascospores of S. mycoparasitica showed higher preference for two-polar germination (FIG. 16G). Single polar germination was also found in F. avenaceum-filtrate suspension; however it was lower than in F. oxysporum-filtrate treatment. Commonly, these single-polar germinated spores produced larger web-like organizations and longer hyphal formation (in F. avenaceum-filtrate suspension), which was rarely found in F. oxysporum-filtrate suspended spores (FIGS. 16H, 16I). The majority of the germinated ascospores in S. mycoparasitica, which was activated through suspension in F. oxysporum-filtrate were detected to be either at an angle of 90° (FIGS. 16E, 16F) or between 90° and 180° (FIG. 16F) at the tip of the polar germ pores. However, very few spores germinated at an angle of 180° (FIG. 16D). Most activated spores (with F. avenaceum-filtrate treatment) showed germination at an angle of 180° (FIG. 16G).

### Example 8

The host specificity of S. mycoparasitica to F. graminearum 3-ADON and 15-ADON strains was assessed in this study.

Media, reagents and chemicals: Potato dextrose agar (PDA), potato dextrose broth (PDB), yeast extract, malt extract agar (MEA), agar, and peptone were purchased from Difco (Becton Dickinson Diagnostics). Streptomycin sulphate, Neomycin sulphate, and other reagents of analytical grade were from Sigma-Aldrich (Oakville, ON, CA). IQ SYBR Green Supermix for real-time PCR reactions was acquired from Bio-Rad Laboratories (Mississauga, ON, CA).
Fungal strains and growth: All phytopathogenic Fusarium strains: Fusarium graminearum 3-ADON (Fgra3) SMCD 2243, and 15-ADON (Fgra15) SMCD 2244 chemotypes, F. avenaceum (Faye) SMCD 2241, F. oxysporum (Foxy) SMCD 2242, F. proliferatum (Fpro) SMCD 2244, F. sporotrichioides (Fspo) SMCD 224; and one mycoparasitic Sphaerodes mycoparasitica SMCD 2220 strain were retrieved from Saskatchewan Collection and Database (SMCD), maintained on PDA amended with antibiotics (100 mg/L streptomycin sulphate and 12 mg/L neomycin sulphate) and used throughout this study.
Spore production and germination assays: Ascospores of S. mycoparasitica were produced on Modified Leonian's agar (MLA), harvested and prepared as previously described. Also, Fusarium spp. filtrates were prepared, S. mycoparasitica spore germination assays in six different Fusarium filtrates were carried out as previously described.

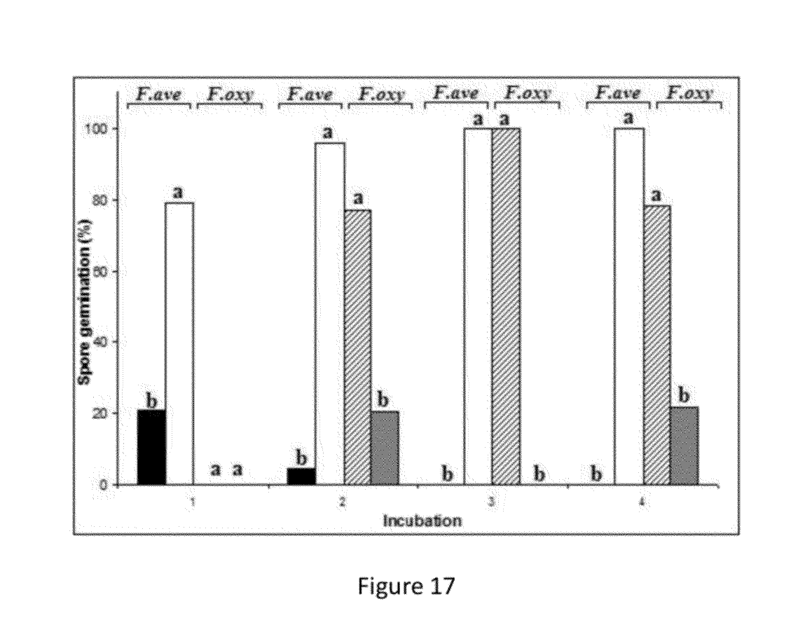

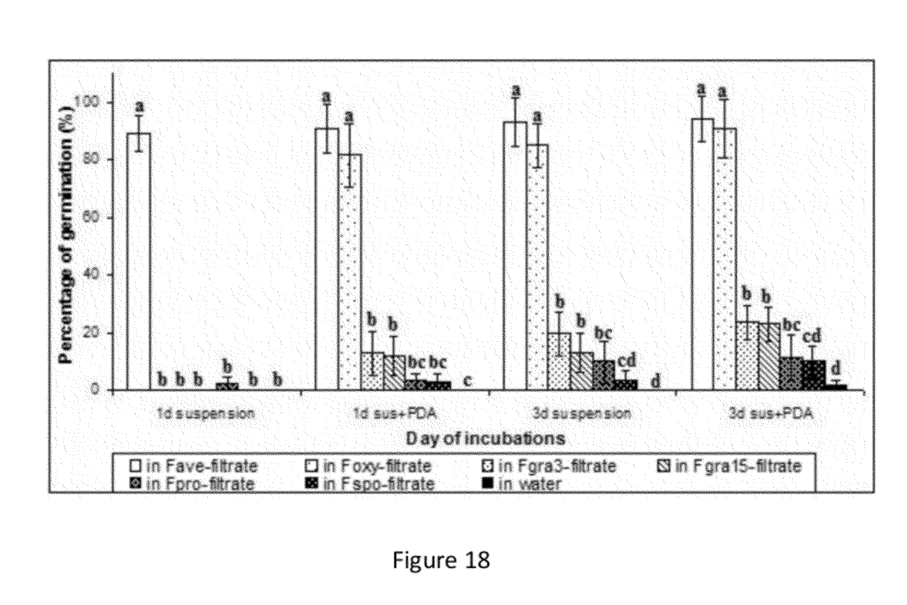

Sphaerodes mycoparasitica spore germination suspended in both F. graminearum chemotype 3-ADON and 15-ADON filtrates was lower compared to F. avenaceum for the first incubation day, and compared to both F. avenaceum and F. oxysporum for the remaining incubation days (P≦0.05; with Mann-Whitney Test) (FIG. 17). No significant differences in germination between F. graminearum, F. proliferatum, and F. sporotrichioides filtrate treatments were observed for the first two incubation days. However, treatments with F. graminearum filtrates showed significantly higher germination rate of S. mycoparasitica compared to F. sporotrichioides filtrate treatment during later incubation time points (FIG. 18).

Dual-culture assays: Dual-culture assays for examining the degree of hyphal reduction/inhibition or damage to F. graminearum chemotypes were assessed as disclosed in Goh and Vujanovic (2009, Mycologia DOI: 10.3852/69-171). S. mycoparasitica is slow-growing fungus as compared to F. graminearum 3-ADON and 15-ADON strains. Therefore, S. mycoparasitica was pre-inoculated onto the PDA plates for 1 d, at 21° C. in darkness, prior to inoculating Fusarium mycelial plugs. Linear mycelial growth of Fusarium strains for both treatments indicated above was measured and recorded daily for 5 days. Around 0.5×1.5 cm² (sampling zones) located approximately 0.2 cm behind the contact zone between F. graminearum and S. mycoparasitica was excised and subjected to DNA extraction. Each treatment was with three replicates and the experiment was repeated twice. The PDA plate inoculated with F. graminearum only was the positive control. Total genomic DNA was extracted using a DNeasy® Plant Mini Kit. The DNA was eluted once in 50 μl of buffer AE and stored at −20° C. until real-time PCR quantification assays (as described below).

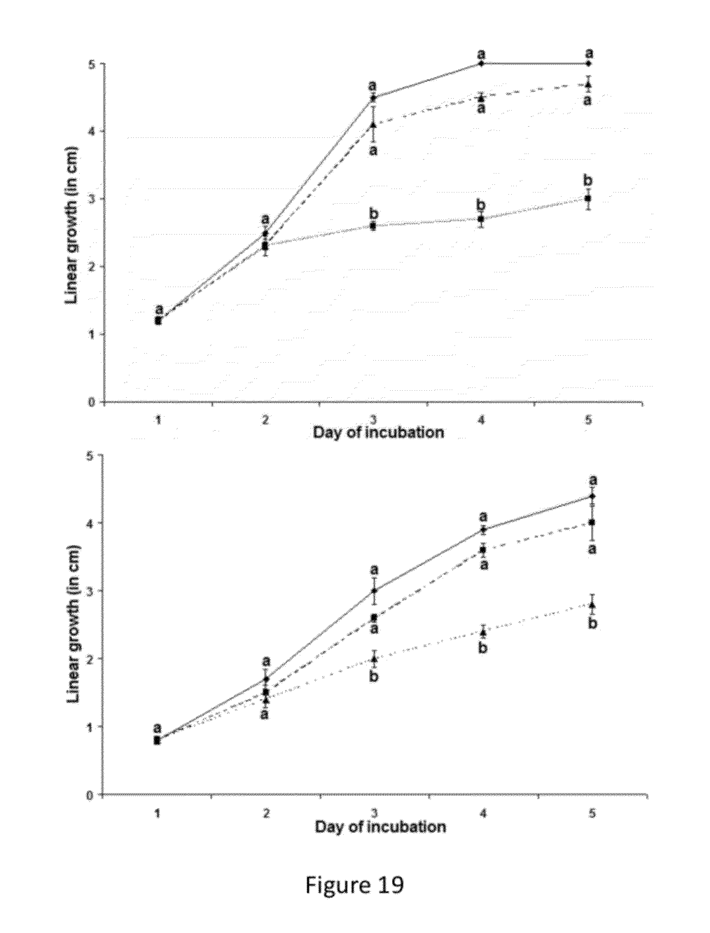

Since S. mycoparasitica demonstrated slower mycelial growth (0.56 cm per day; n=9) compared to F. graminearum 3-ADON (0.74 cm per day; n=6) and 15-ADON (0.68 cm per day; n=6) chemotypes, the linear growth of F. graminearum mycelia in dual-culture was assessed using the pre-inoculation method. S. mycoparasitica was pre-inoculated on PDA for 1 d followed by F. graminearum inoculation. The pre-inoculation approach demonstrated significant differences (starting day 3) in linear growth suppression of F. graminearum chemotype 3 and 15 compared to the co-inoculation approach (FIGS. 19A and 19B).

Establishment of mycoparasitism: Fusion biotrophic mycoparasitic interactions between S. mycoparasitica and both F. graminearum chemotype strains, and intracellular parasitism interactions were examined and assessed on slide cultures according the methods described in Goh & Vujanovic (2009).

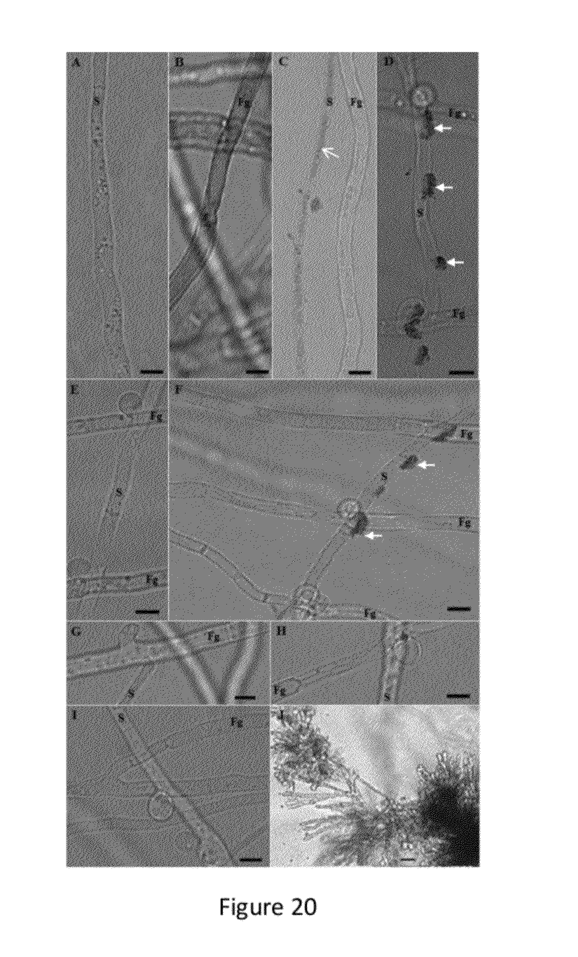

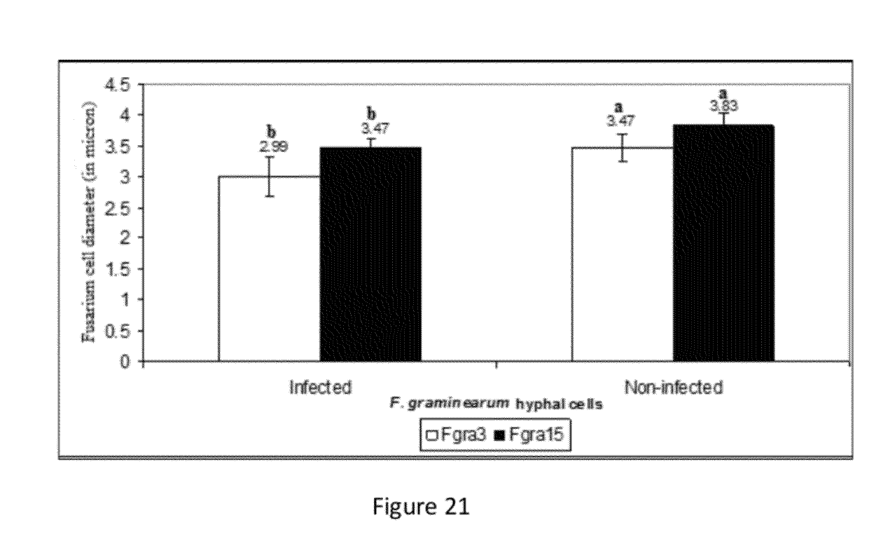

On day 3 of inoculation on PDA with F. graminearum 3-ADON and 15-ADON, no clamp-like or hook-like structures were formed by Sphaerodes mycoparasitica on the Fusarium strains. On day 5 of inoculation, clamp-like and hook-like contact structures as well as Fusarium hyphal cells penetration (with haustoria) were observed (FIGS. 20E to 20I). Furthermore, on day 3, S. mycoparasitica removed red pigment from the mycelia of F. graminearum 3-ADON on the slide culture (FIGS. 20A to 20D). As a result, S. mycoparasitica mycelia adopted a reddish color (FIG. 20C). Between day 4 and 5, formation of red crystal-like pellets was detected on the surface of mycoparasite hyphae (FIG. 20D). The mechanism behind the color changes observed remains unknown. For F. graminearum chemotype 15-ADON, no uptake of red complex or release of red crystal-like structures by S. mycoparasitica hyphae were noted. Nevertheless, flower-like hyphal structures appeared which could indicate possible growth inhibition of 15-ADON F. graminearum (FIG. 20J). Significant differences in diameters of infected and non-infected hyphae were seen for both F. graminearum chemotypes (FIG. 21).

Primers and standard curves: F. graminearum-specific (Fg16NF/R) and trichothecene Tri5 gene-specific (Tox5-1/2) primer sets were used in this study. Standard curves for F. graminearum- and Tri5 gene-primer sets were generated, based on threshold cycles (Ct), by using a series of 10-fold diluted genomic DNAs from F. graminearum (spanning from 2.7×10² to 2.7×10⁻² ng/μl for F. graminearum-specific primer set, from 2.7×10² to 2.7×10⁴ ng/μl of 3-ADON strain and 3.0×10¹ to 3.0×10⁻² ng/μl of 15-ADON strain DNAs for Tox5-1/2 primer set). Ct values were recorded and obtained by the Opticon Monitor software version 3.1 (Bio-Rad Laboratories Inc., Mississauga, ON, CA). Standard curves for different primer sets were constructed by plotting the threshold cycles (Ct) value versus the logarithm (log₁₀) of the concentration of 10-fold serial diluted F. graminearum DNAs as described above. Amplifications with different primer sets on the genomic DNAs of two F. graminearum chemotypes were run in triplicates to obtain the mean and standard deviation of each 10-fold serial dilution.

Real-time PCR quantification: Real-time PCR amplifications on total genomic DNA extracted from the sampling zones (as described above) were performed using MiniOpticon (Bio-Rad Laboratories Inc., Mississauga, ON, CA). All the real-time PCR reactions were performed by utilizing the real-time PCR MJ white tubes (Bio-Rad Laboratories Inc., Mississauga, ON, CA) in a total volume of 25 μl. The reaction mixture for all real-time PCR assays were: 12.5 μl of IQ Supermix (Bio-Rad Laboratories Inc., Mississauga, ON, CA), 1 μl of each 10 μM forward/reverse primers (Invitrogen), 9.5 μl of sterilized UltraPure Millipore water, and 1 μl of DNA template. Real-time PCR conditions for Fg16NF/R primer set used were outlined in Nicholson et al. (1998, Physiol. Mol. Plant. Pathol. 53: 17-37) with melting curve analysis at 60 to 95° C. Parameters for Tox5-1/2 primer set as described in Schnerr et al. (2001, Int. J. Food Microbiol. 71: 53-61).

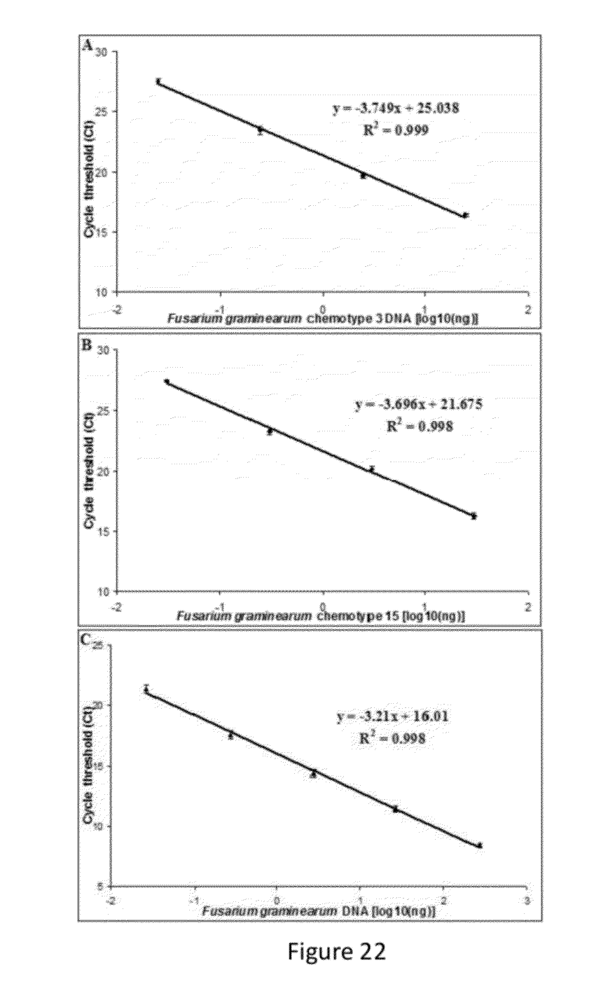

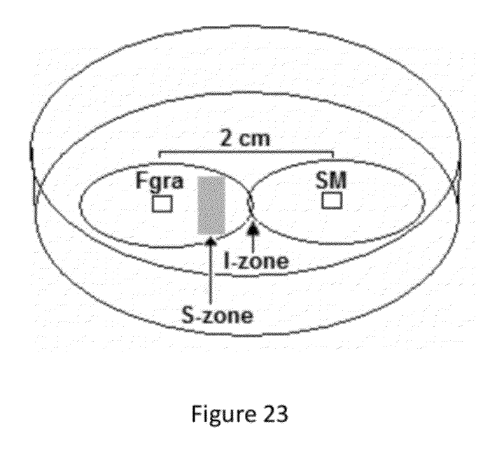

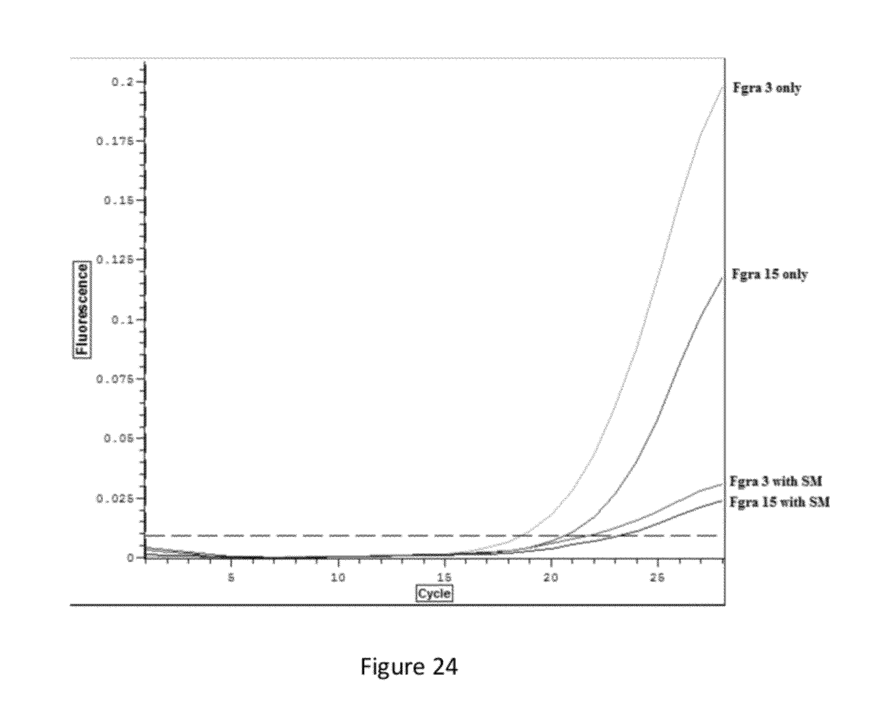

Standard curves for different primer sets with different F. graminearum DNA sources were constructed (FIG. 22). Growth suppression or inhibition at the sampling zones (FIG. 23) for F. graminearum chemotype 3-ADON and 15-ADON was further confirmed with real-time PCR amplifications with F. graminearum- and tri5 gene-specific primer sets. Sigmoidal curves for the four different treatments (F. graminearum chemotype 3 or 15 only and F. graminearum chemotype 3-ADON or 15-ADON pre-inoculated with S. mycoparasitica) with Fg16NF/R primer set were generated using Opticon Monitor software version 3.1 and illustrated in FIG. 24.

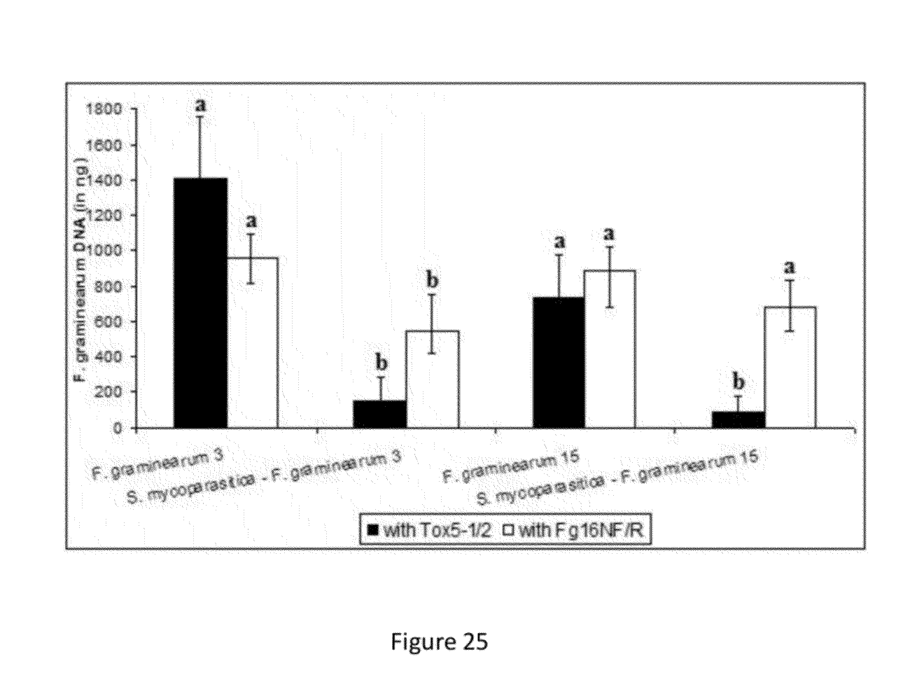

Using Fg16NF/R primer set, the quantity of F. graminearum chemotype 3-ADON DNA in the sampling zones significantly decreased when pre-inoculated with S. mycoparasitica compared to the non-inoculated treatment (P=0.01) (FIG. 25). DNA of F. graminearum chemotype 15-ADON was considerably but not significantly reduced (P=0.085 using T-test). Using Tox5-1/2 primer set, amount of tri5 gene fragments diminished appreciably in both F. graminearum chemotype 3-ADON and 15-ADON challenged with S. mycoparasitica (P≦0.05).

### Example 9

The effects of S. mycoparasitica (biotrophic mycoparasite) and T. harzianum (necrotrophic mycoparasite) on expression of Tri and PKS genes by F. graminaerum 3-ADON and 15-ADON strains were assessed in this study.

Fungal strains and growth: Two Fusarium graminearum 3-ADON (SMCD 2243) and 15-ADON (SMCD 2244) chemotypes, Trichoderma harzianum necrotrophic (SMCD 2166) and Sphaerodes mycoparasitica biotrophic (SMCD 2220) mycoparasites were obtained from the Saskatchewan Microbial Collection and Database (SMCD). All strains were maintained on Potato dextrose agar (PDA, BD Biosciences, Mississauga, ON, CA) amended with antibiotics (100 mg/L streptomycin sulphate and 12 mg/L neomycin sulphate; Sigma-Aldrich Canada Ltd., Oakville, ON, CA) prior to study initiation.

Chemical fungicide control: A concentration of 100 μmol/L tebuconazole was prepared from Folicur® 432F (43.2% tebuconazole, Bayer CropScience Inc., Saskatoon, SK, CA; Folicur is a registered trademark of Bayer Aktiengesellschaft, Leverkusen, Fed. Rep. Germany). This fungicide preparation was used throughout the study.
In vitro assay, sampling and RNA extraction: Dual-culture assay was carried out between F. graminearum and Folicur® (100 mol/L tebuconazole) or biological (T. harzianum or S. mycoparasitica) agents on Minimal medium as disclosed by Xue et al. (2009, Can. J. Plant Pathol. 31: 169-179). Inoculated dual-culture plates were incubated at room temperature (23° C.) in dark conditions for a week. Mycelia of F. graminearum were harvested after a week of inoculation with either chemical or biological agents. A 0.5-cm² mycelial plug was cut out approximately 0.5 cm away from the border of interaction and fungal cells were disrupted with liquid nitrogen. Total RNA from the samples were extracted using Aurum Total RNA mini kit and extracted RNA was treated with DNAse (Bio-Rad Laboratories Inc., Mississauga, ON, CA) according the manufacturer's recommendations. Samples were then stored at −70° C. until gene expression analysis.

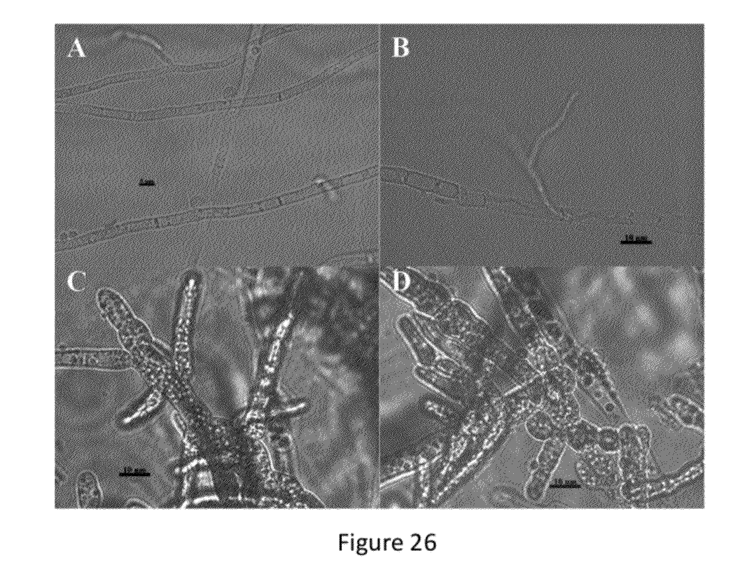

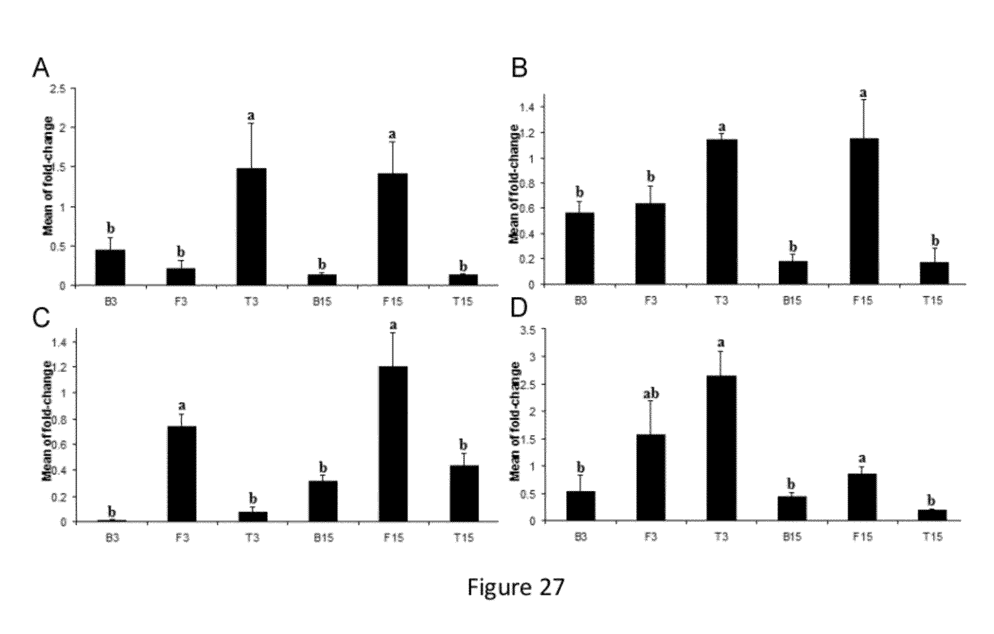

During the in vitro studies, both F. graminearum 3-ADON and 15-ADON chemotypes changed mycelia morphology corresponding to each treatment (FIGS. 26A-26B). However, the main diagnostic distinction was the tendency of F. graminearum chemotypes to abundantly produce chlamidospores in clusters or chains when exposed to tebuconazole fungicide compared to mycoparasites (FIGS. 26C-26D). In the case of F. graminearum chemotype 15 (15-ADON producer), all four trichothecene genes—Tri4, Tri5, Tri6, and Tri10 in this Fusarium strain were found to be induced in high amounts when treated with chemical tebuconazole compared to treatments that were co-inoculated with a biological agent (either S. mycoparasitica or T. harzianum) (FIGS. 27A-27D). F. graminearum chemotype 3 (3-ADON producer) was observed to demonstrate induction of Tri4, Tri5 and Tri10 genes when co-inoculated with necrotrophic mycoparasitic Trichoderma harzianum (FIGS. 27A, 27B, 27D). When F. graminearum chemotype 3-ADON was treated with Folicur® fungicide, only Tri10 gene was being induced (FIG. 27D). Generally, all four Tri genes were repressed significantly when challenged with biotrophic mycoparasitic Sphaerodes mycoparasitica (FIG. 27A-27D).

Real-time Reverse-transcription PCR: Real-time RT-PCR was performed by using an IScript One-Step RT-PCR kit with SYBR Green on a MiniOpticon Cycler System (Bio-Rad Laboratories Inc., Mississauga, ON, CA), according to the manufacturer's instruction. Primer sets used for amplification and gene expression are summarized in Table 6.

TABLE 6
Primer sets used to amplify Tri4, Tri5, Tri6, Tri10, PKS4, PKS13,
and β-tubulin by Real-time PCR.

| SEQ ID | Gene | Primer sequences | Reference |
|---|---|---|---|
| SEQ ID NO: 6/7 | Tri4 | Tri4-F: TAAACGCCCGCGAAGTTCACA | Jiao et al. 2008, FEMS Lett. |
| | | Tri4-R: TGGTGATGGTTCGCTTCGAG | 285: 212-219 |
| SEQ ID NO: 8/9 | Tri5 | Tr5F: AGCGACTACAGGCTTCCCTC | Doohan et al. 1999, Appl. |
| | | Tr5R: AAACCATCCAGTTCTCCATCTG | Environ. Microbiol. 65: |
| | | | 3850-3854 |
| SEQ ID NO: 10/11 | Tri6 | Tri6-1 TCTCTACCAACGGTGGATTCAACC | Pinson-Gadais et al. 2008, |
| | | Tri6-2 AGCCTTTGGTGCCGACTTCTTG | Mycopathol. 165: 51-59. |
| SEQ ID NO: 12/13 | Tri10 | Tri10-F: TCTGAACAGGCGATGGTATGGA | Jiao et al. 2008 |
| | | Tri10-R: CTGCGGCGAGTGAGTTTGACA | |
| SEQ ID NO: 14/15 | PKS4 | PKS4-PS.1 | Lysøe et al. 2006, Appl. |
| | | GTGGGCTTCGCTAGACCGTGAGTT | Environ. Microbiol. |
| | | PKS4-PS.2 | 72: 3924-3932 |
| | | ATGCCCTGATGAAGAGTTTGAT | |
| SEQ ID NO: 16/17 | PKS13 | PKS13-PS.1 | Lysøe et al. 2006 |
| | | CCCCCAACTCGACGTCAAATCTAT | |
| | | PKS13-PS.2 | |
| | | TTCTTCCCGCCGACTTCAAAACA | |
| SEQ ID NO: 18/19 | β- | FGtubf GGTCTCGACAGCAATGGTGTT | Lysøe et al. 2006 |
| | tubulin | FGtubr GCTTGTGTTTTTCGTGGCAGT | |

RT-PCR sample (˜25 μL) contained 3 μL of RNA template, 8.85 μL of nuclease-free water, 12.5 μL of RT-PCR reaction mixture (2×), 0.5 μL of IScript RT enzyme mix (50×) (Bio-Rad Laboratories Inc., Mississauga, ON, CA), and 0.1 μL of 50 μM solutions of both forward and reverse targeted gene-specific primers (Invitrogen Corp., Carlsbad, Calif., USA). Real-time PCR conditions were performed as outlined by manufacturer's recommendations: 50° C. for 10 min, 95° C. for 5 min, followed by 40 cycles of denaturing at 95° C. for 10 s and annealing at 55° C. for 30s, 95° C. for 1 min and 55° C. for 1 min. PCR reactions were checked for absence of any primer-dimer formation or non-specific PCR amplification by performing melting curve analysis. Contamination of RNA template with residual genomic DNA was eliminated because there was no amplification detected using reverse transcriptase free real-time RT-PCR reaction as template. Fold change in gene expression for each treatment was normalized to β-tubulin internal reference gene and relative to the expression for the control treatment (Fusarium alone on minimal medium), using the 2^{-ΔΔCT} method proposed by Livak and Schmittgen (2001), Methods, 25(4): 402-408). ΔΔC_{T}=(C_{T, target-gene}−C_{T, Fgtub})ₜᵣₑₐₜₘₑₙₜ−(C_{T, target-gene}−C_{T, Fgtub})_{control}, where treatment and control indicate F. graminearum challenged with chemical or biological agent and F. graminearum alone, respectively.

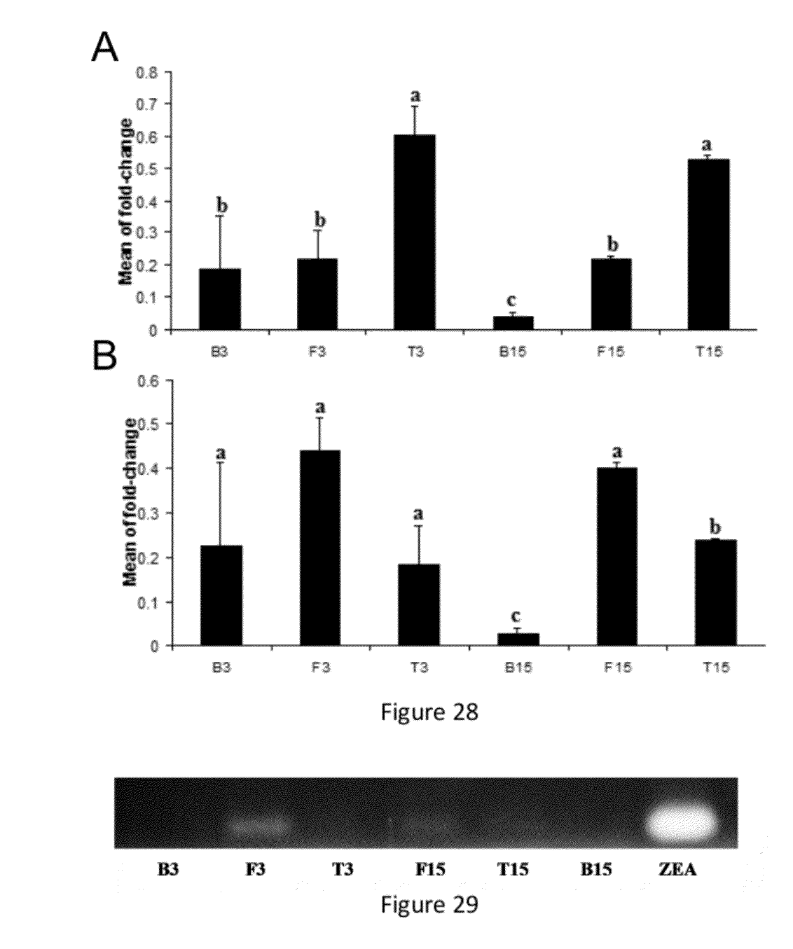

For gene expression analyses based on targeted genes (PKS4 and PKS13) that are responsible for zearalenone biosynthesis, these two polyketide synthase genes from both F. graminearum chemotypes were detected to be repressed in all chemical or biological treatments (FIGS. 28A-28B). Repression of F. graminearum chemotype 15 PKS4 and PKS13 genes in the treatment with S. mycoparasitica biotrophic mycoparasite was illustrated to be significantly higher compared to treatments with T. harzianum and tebuconazole fungicide (FIGS. 28A-28B).

Tri genes were observed to be more sensitive in F. graminearum chemotype 3-ADON when co-inoculated with Trichoderma necrotrophic mycoparasite, however, in F. graminearum chemotype 15-ADON, Tri genes appeared to be more responsive towards treatment with chemical fungicide (FIGS. 27A-27D).

PKS4 gene for both F. graminearum chemotypes was monitored to be more sensitive towards treatments with Trichoderma necrotrophic mycoparasitic fungus compared to chemical and biotrophic mycoparasitic agents (FIG. 28A). However, PKS13 gene in F. graminearum chemotype 15-ADON was found to demonstrate higher sensitivity towards chemical stimulus (FIG. 28B).

Mycotoxins extraction and analyses: DON, ZEA, 3-ADON and 15-ADON mycotoxins were extracted from agar mycelial plugs (0.5 cm²) cut from the sampling zone located approximately 0.5 cm behind the contact zone between F. graminearum and S. mycoparasitica. The extraction was performed by three 10-ml volumes of ethyl acetate. The samples were sonicated on ice and shaken vigorously in ethyl acetate, and then they were allowed to stand for 5 min for separation of phases. The organic phase was siphoned off and passed through sodium sulfate to remove water. The solvent was allowed to evaporate at room temperature (23° C.) for 3 days. The residue was then re-dissolved in 2 ml of acetonitrile for thin liquid chromatography (TLC) (only ZEA) and high performance liquid chromatography (HPLC) (all mycotoxins) analyses. TLC was performed on Silica gel 60 plates (Merck Frosst Canada Ltd, Kirkland, PQ, CA) in the solvent system with ethyl acetate-toluene (3:1) for 40 min. 3-ADON on the gel plates was sprayed with 20% aluminum chloride in 95% ethanol, detected and analyzed following the protocols disclosed by Vasavada and Hsieh (1987, Appl. Microbiol. Biotech. 26: 517-521). Known standard controls of pure DON, ZEA, 3-ADON and 15-ADON (Sigma-Aldrich Canada Ltd., Oakville, ON, CA) were employed to compare with other treatments. All four mycotoxins were quantified using a Water's 2695 HPLC system with: 250×4.60 mm, Luna 5 μL micron C18 (2) 100A column (Phenomenex Inc., Torrance, Calif. USA) and a photodiode-array (PDA) detector was used with an isocratic solvent system (methanol: water-methanol containing 5% (v/v) (90:10) ratio). The PDA detector measured the UV spectrum (190-500 nm). Samples were dissolved in acetonitrile and 10 μl loaded onto the column using automatic injector and mycotoxins were eluted with solvent as a mobile phase at a rate of 0.75 ml min⁻¹ for 25 mins. Peak height method was incorporated to determine the exact amount of DON, ZEA, 3-ADON and 15-ADON against a standard curve. Ratio between mycotoxins extracted from treatments of F. graminearum treated with Folicur and F. graminearum only was calculated for HPLC.

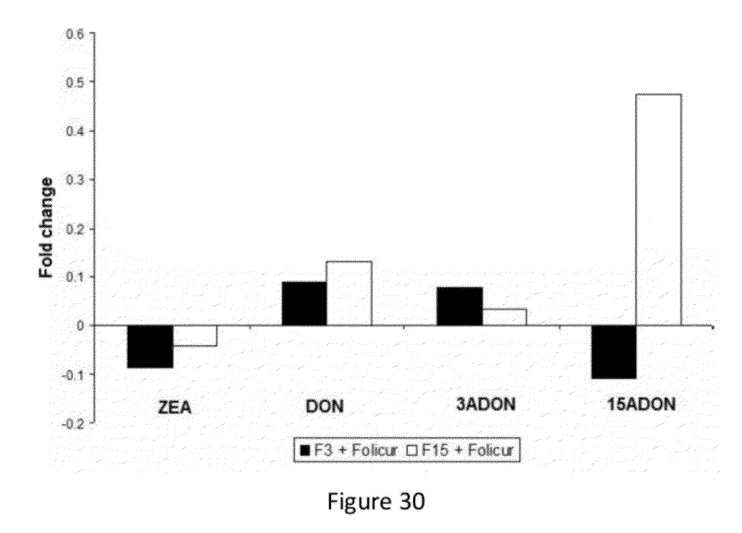

The amount of DON produced was between 70 to 90 μg/L. Consequently, it was too low to be detected in TLC analysis. ZEA was high enough to be analyzed with TLC and both F. graminearum chemotypes were observed to produce higher amounts of ZEA toxin under the treatment with Folicur® compared to other treatments (FIG. 29). With HPLC, ZEA was found to be reduced when challenged with Folicur® compared to F. graminearum colony only (FIG. 30). DON and 3-ADON were detected to increase for both F. graminearum when inoculated together with Folicur® (FIG. 30). Folicur® was monitored to trigger the highest amount of 15-ADON under in vitro assay (FIG. 30).

### Example 10

The effects of S. mycoparasitica (biotrophic mycoparasite) and T. harzianum (necrotrophic mycoparasite) on expression of aurofusarin gene by F. graminearum 3-ADON and 15-ADON strains were assessed in this study.

Fungi, media, and culture conditions: Fungal strains were obtained from culture collections at University of Saskatchewan (Food and Bioproduct Sciences fungal collection). Strains of the following fungal species were used, plant pathogenic Fusarium graminearum, F. graminearum (3-ADON), F. graminearum (15-ADON), F. avenaceum, F. culmorum, F. proliferatum, F. oxysporum, F. arthrosporoides and mycoparasitic Spaeherodes mycoparasitica and Trichoderma harzianum. Fungi were maintained on potato dextrose agar for 2 wks at 21° C. in darkness. Folicur® (43.2% tebuconazole, Bayer CropScience Inc., Saskatoon, SK, CA; Folicur is a registered trademark of Bayer Aktiengesellschaft, Leverkusen, Fed. Rep. Germany) was also used in this study. Experiments for the impact of red color nuances on aurofusarin gene expression was carried out using previously classified F. avenaceum isolates, based on VCGs (vegetative compatibility groups) and colony colours. F. avenaceum isolates color identification numbers (CIN) were generated with Hex Color Code Chart (Abdellatif et al. 2010. Canadian Journal of Plant Pathology 32(: 468-480). CINs are: Ds #71232B: Red and highly virulent, tolerant at 80° C. for 4 hours; Es #4E040B: Moderately red and moderately virulent, tolerant 40° C. for 4 hours; Bs #A86608: White and non virulent, susceptible 40° C. for 4 hours. Three 0.1 g mycelium samples from three replicates of each isolate was mixed and used for DNA extraction and RT-PCR analyses.
Fungal DNA Extraction and PCR: Fungal cultures were grown on 1.5 ml PD broth and centrifuged at 10,000 rpm for 5 min. Supernatant was discarded and, subsequently a total DNA of 2-week old cultures representative of each phenotype/VCG was extracted from the pellet with an Ultra Clean microbial DNA Isolation Kit (Qiagen Inc., Mississauga, ON, CA) following manufacture's instructions. The purified DNA was resuspended in 50 μl of elution buffer and stored at −20° C. until further analyses. Full length genomic sequences of PKS region (7.2 Kb) from Fusarium graminearum were obtained from NCBI Genbank. Six pairs of primers were designed (Table 7).

TABLE 7
PCR Primers designed from full-length genomic
sequences of the PKS region of F. graminearum

| SEQ ID | Primer | Sequence |
|---|---|---|
| SEQ ID NO: 20 | PKSF1 | TCGAGTTTCGTGTTGCGTGT |
| SEQ ID NO. 21 | PKSR1 | AGGTAGTTCGCCATACCCGT |
| SEQ ID NO: 22 | PKSF2 | AATTGTGCCCGAGGCAGTAC |
| SEQ ID NO: 23 | PKSR2 | CATTGGTTCCGCCCGCAATAG |
| SEQ ID NO: 24 | PKSF3 | TGACAACTTCGCTGGTTTGGA |
| SEQ ID NO: 25 | PKSR3 | CATAGCTTGGCCAGTGCCATC |
| SEQ ID NO: 26 | PKSF4 | GAAGTCATTCGGTGTTGAGC |
| SEQ ID NO: 27 | PKSR4 | GCTCTGGATTGGGTATCGCAC |
| SEQ ID NO: 28 | PKSF5 | ACTCGAGCATCCGTCGCAATG |
| SEQ ID NO: 29 | PKSR5 | AGCAACATCTCCGTCTGGAG |
| SEQ ID NO: 30 | PKSF6 | GTTGAACTGTCCATGGCTGA |
| SEQ ID NO: 31 | PKSR6 | GAATGAAGGCAATCTGCTGC |

All the reactions were carried out in 25-μl volumes containing 2.5 μl of 10×PCR buffer, 5 μl of Q solution, 1 μl of each primer (10 μM), 0.5 μl of 10 mM dNTPs mix, and 1.25 U of Taq polymerase (Qiagen Inc., Mississauga, ON, CA). Reaction mixtures were mixed gently and were given flash spin prior to PCR in an Eppendorf Master Cycler (ep gradient S). PCR amplicons were purified using the QIAquick® PCR purification kit (Qiagen Inc., Mississauga, ON, CA; QIAquick is a registered trademark of Qiagen GmbH Corp., Hilden, Fed. Rep. Germany) and commercially sequenced (Plant Biotechnology Institute, Saskatoon, SK).

Development of a real time RT-PCR assay for detection and quantification of the aur gene in Fusarium species: All products were sequenced, sequences were aligned, several primer sets were designed and tested. One set, named Auro RT (SEQ ID NO: 32) and Auro RTR (SEQ ID NO: 33) was selected based on efficiency to amplify aurofusarin gene.

SEQ ID NO: 32
ACCTCACTGGAATCAGAGCGCAGC
SEQ ID NO: 33
ATGACRACTTCCCGTGGRCC

The specificity of this set of primers was tested by conventional PCR assay using genomic DNA from Fusarium and mycoparasites using the amplification reactions volumes indicated above. The amplification protocol was 1 cycle of 120 s at 94° C., 35 cycles of 30 s at 94° C. (denaturation), 30 s at 56° C. (annealing), 45 s at 72° C. (extension), and 1 cycle of 10 min at 72° C.

The pair of primers used to amplify the β-tubulin (tub) gene (the endogenous control gene used to normalize the results) were FGtubf (SEQ ID NO: 34) and Fgtubr (SEQ ID NO: 35).

SEQ ID NO: 34
GGTCTCGACAGCAATGGTGTT
SEQ ID NO: 35
GCTTGTGTTTTTCGTGGCAGT

The PCR efficiencies of the real time RT-PCR for both genes were checked by performing a 10-fold serial dilution of positive control template to generate a standard curve, and by plotting the Ct as a function of log₁₀ of template.

Presence of aurofusarin was tested and quantified by RT-PCR in pathogenic Fusarium graminearum, F. graminearum (3-ADON), F. graminearum (15-ADON), F. avenaceum, F. culmorum, F. proliferatum, F. oxysporum, F. arthrosporoides and mycoparasitic Sphaerodes mycoparasitica and Trichoderma harzianum. Relative gene expression was studied when Fusarium isolates were co-cultured with Folicur®, Sphaerodes and Trichoderma.

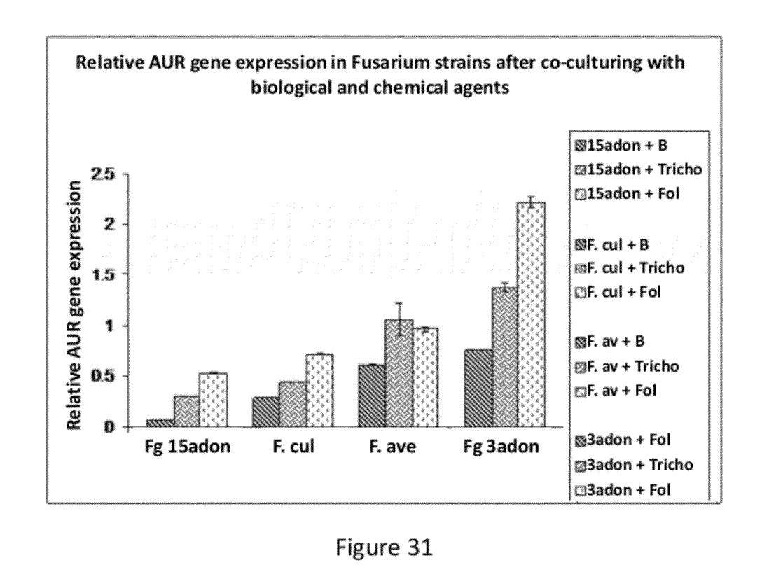

The designed RT-PCR primer set was checked for its specificity by conventional PCR on tested fungal species. A single band 157 bp long was amplified in F. graminearum (3-ADON), F. graminearum (15-ADON), F. avenaceum, and F. culmorum but not in other strains. One week old grown cultures tested for their aurofusarin relative gene expression and β-tubulin gene was used as an internal control. F. graminearum which produces 15-ADON showed highest level gene expression followed by F. graminearum (3-ADON) and similar expression results were observed with F. avenacum and F. culmorum. Aurofusarin relative gene expression was quantified when each Fusarium species was co-cultured for a week with Sphaerodes, Trichoderma and Folicur® (FIG. 31). Among all tested, S. mycoparasitica was most effective in reducing the aurofusarin relative gene expression followed by Trichoderma and Folicur® (FIG. 31). S. mycoparasitica was most affective on F. graminearum producing 15-ADON, but also efficiently reduced the expression level in F. culmorum and F. avenaceum and F. graminearum producing 3-ADON (FIG. 31). Similarly, Trichoderma was able to somewhat affect F. graminearum producing 15-ADON and F. culmorum, but showed no impact on F. avenaceum, whereas enhanced the expression in F. graminearum producing 3-ADON (FIG. 31). Finally, Folicur® could be able to provoke minor reduction in F. graminearum producing 15-ADON aur gene expression, but with less impact on F. culmorum and F. avenaceum. Inversely, Folicur® increased expression almost 2-fold when compared to F. graminearum producing 3-ADON (FIG. 31).

RNA isolation, reverse transcription and real time RT-PCR: All the cultures were grown for a week under dark conditions and used for expression studies. Fungal total RNA was isolated using the “Total Quick RNA Cells and Tissues” Kit (Bio-Rad Laboratories Inc., Mississauga, ON, CA), according to the manufacturer's instructions, and stored at −80° C. DNAse I treatment to remove the chromosomal DNA contamination from the samples was performed using the “Deoxyribonuclease I, Amplification Grade” (Invitrogen Corp., Carlsbad, Calif., USA), First strand cDNA was synthesized using the “Iscript RNA PCR Reagent Kit” (Bio-Rad Laboratories Inc., Mississauga, ON, CA). Relative quantification of aur gene expression was performed in a MiniOpticon Sequence Detection System using the SYBR Green PCR Master Mix (Bio-Rad Laboratories Inc., Mississauga, ON, CA) and the primer pairs indicated above. The PCR thermal cycling conditions for both genes were as follows: an initial step at 95° C. for 10 min and 40 cycles at 95° C. for 15 s and at 60° C. for 1 min. SYBR green PCR master mix 12.5 μl (Bio-Rad Laboratories Inc., Mississauga, ON, CA) was used as the reaction mixture, with the addition of 6.5 μl of sterile milli-Q water, 1.0 μl of each primer (5 μM), and 5 μl of template cDNA, in a final volume of 25 μl. In all the experiments, appropriate negative controls containing no template were subjected to the same procedure to exclude or detect any possible contamination or carryover. Each sample (triplicate) was amplified twice in every experiment. The results were normalized using the all fungi cDNA amplifications run on the same plate. The tub2 gene is an endogenous control that was used to normalize quantitation of mRNA target for differences in the amount of total cDNA added to each reaction. Real Time RT-PCR analysis is based on the threshold cycle (CT), which is defined as the first amplification cycle at which the fluorescence signal is greater than the minimal detection level, indicating that PCR products become detectable. Relative quantitation was the analytical method used in this study. A comparison within a sample is made with the gene of interest (aur) to that of the endogenous control gene (tub2). Quantitation is relative to the control gene by subtracting the CT of the control gene (tub2) from the CT of the gene of interest (aur) (ΔCT). Each ΔCT value (corresponding to each sample) was subtracted by the calibrator value (FpMM6-1C) to obtain the corresponding ΔΔCT values. ΔΔCT values were transformed to log₂ (due to the doubling function of PCR) to generate the relative expression levels.

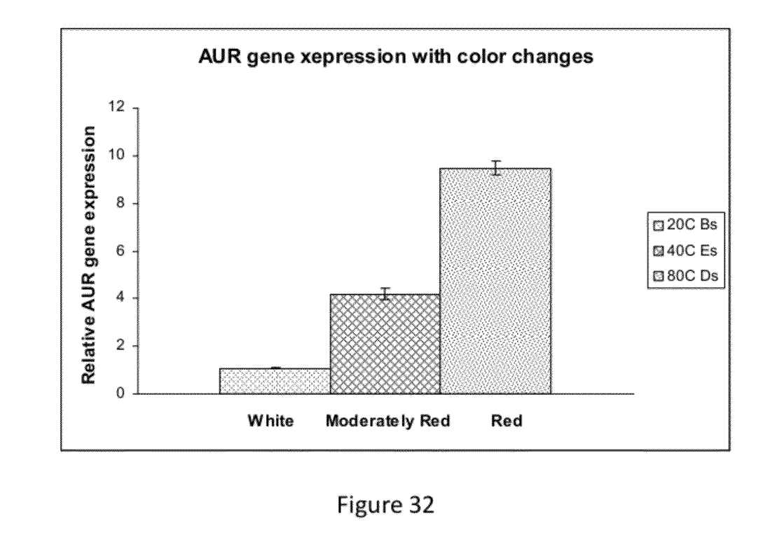

In total, 9 isolates were subjected to RT-PCR analyses for quantitative gene expression analyses. Three kinds of colored samples were used in this study composed of 3 white colored, 3 moderately red colored and 3 dark red colored isolates. White colored isolates were used as control and their expression was normalized and taken as one. As reported moderately red colored isolates were able to tolerate 40° C. whereas; dark red isolates were able to tolerate 80° C. The RT-PCR results show that dark colored isolates had almost 9-fold enhanced gene expression, whereas moderately red colored isolates had 4 fold-enhanced expression, relative to the white colored isolate control (FIG. 32).

### Example 11

Greenhouse studies were performed to assess the effects of S. mycoparasitica inoculants on development of Fusarium head blight symptoms and the accumulation of tricothecene mycotoxin gene in wheat and barley spikes.

Fungal isolates and growth: S. mycoparasitica SMCD 2220-01, and F. graminearum 3-ADON chemotype SMCD 2243 were obtained from the Saskatchewan Microbial Collection and Database (SMCD). These fungal cultures were grown on potato dextrose agar (PDA, BD Biosciences, Mississauga, ON, CA) supplemented with antibiotics prior to the study. Mycelial suspensions of these fungal strains (10⁴ CFU/mL for F. graminearum and 10⁶ CFU/mL for S. mycoparasitica) were produced for greenhouse experiment, as follows.

F. graminearum 3-ADON chemotype strain was inoculated in potato dextrose broth (PDA, BD Biosciences, Mississauga, ON, CA), incubated at room temperature (˜21° C.) in darkness using shaker (100 rpm), for a week prior to harvesting the mycelia for greenhouse application. Mycelia of F. graminearum were transferred into sterilized commercial blender for cutting the mycelial inoculants into small debris and these mycelia were adjusted to concentration of 10⁴ CFU/mL.

S. mycoparasitica was inoculated in yeast peptone dextrose (YPD) (yeast, 5 g; peptone, 10 g; dextrose, 10 g per 1 L of sterile-double distilled water) broth, incubated at room temp in darkness, using shaker at 100 rpm for a week prior to harvesting the mycelia. Mycelia of S. mycoparasitica were transferred into a sterilized commercial blender for cutting the mycelial inoculants into small debris and the mycelia were adjusted to a concentration of 10⁴ CFU/mL for greenhouse application.

Greenhouse trials: The Fusarium-susceptible wheat CDC-TEAL and barley BOLD cultivars were used to test the efficacy of the biotrophic mycoparasite S. mycoparasitica and one potential Fusarium antagonistic fungal isolate as potential biocontrol agents for managing Fusarium head blight diseases. Surface-sterilized wheat and barley seeds were planted in 15-cm diameter pots containing Pro-Mix® soil (Sun Gro Horticulture, Delta, BC, CA) and maintained at 23-25° C. during the day time and 18-20° C. during the night time in a greenhouse with light intensity of 360 μmol m⁻² s⁻¹, watered every 2 d, and fertilized every 14 d using 1300 ppm of NPK (20-20-20) fertilizer. All treatments in the experiment were with three replicates and repeated twice.

At the anthesis stage, wheat and barley spikes were sprayed with a mycelial suspension (2 mL/spike) at a concentration of 10⁶ CFU/mL and 10⁴ CFU/mL of S. mycoparasitica biocontrol agent and Fusarium graminearum pathogenic isolate, respectively; 65 μmol/L tebuconazole, or sterile distilled water. The treatments were applied as outlined in Xue et al. (2009, Can. J. Plant Pathol. 31: 169-179) with slight modifications. Following the inoculation of S. mycoparasitica, plants were kept at room temperature and covered with sterile Whirl-Pak® bags (Whirl-Pak is a registered trademark of Aristotle Corp., Stamford, Conn., USA) overnight to allow fungal growth prior to F. graminearum inoculation. Spikes were then inoculated with mycelial suspension of F. graminearum at a concentration of 10⁴ CFU/mL. F. graminearum inoculum was sprayed on the spikes and covered with sterile Whirl-Pak® bags for overnight. Spikes inoculated with S. mycoparasitica alone and F. graminearum alone, served as positive and negative control, respectively. The inoculated plants were maintained for an additional 21 days prior to sampling. Percentage of infected spikelets (1S) per spike, FHB index, FDK, and weight of 100 seeds were rated as outlined in Xue et al. (2009, Canadian Journal of Plant Pathology 31: 169-179). The FHB visual severity scale was determined according to the methods disclosed by Stack and McMullen (2011, PP-1095 online: http://www.ag.ndsu.edu/pubs/plantsci/smgrains/pp 1095w.htm).

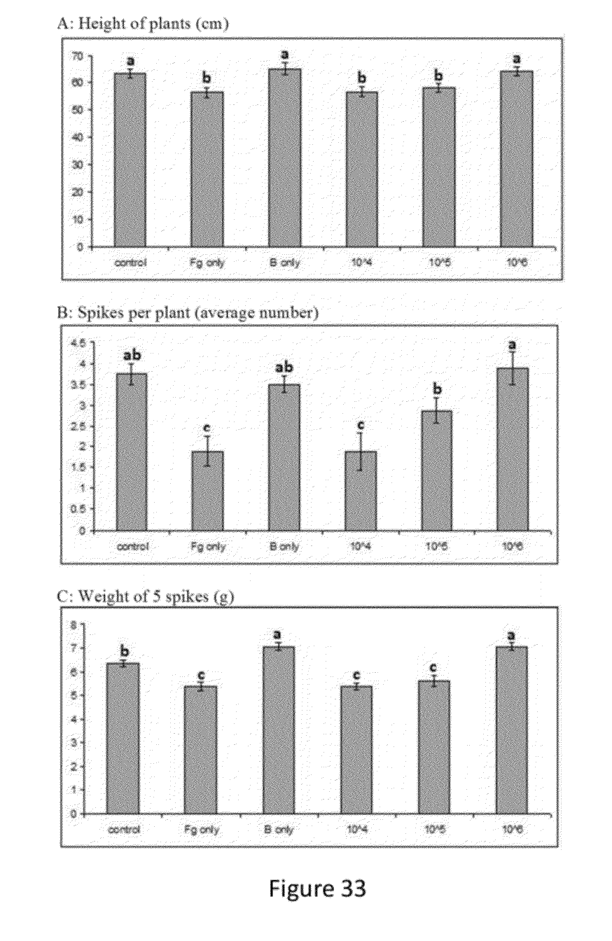

The biocontrol effects of different concentrations of S. mycoparasitica of Fusarium head blight symptoms in barley are shown in FIG. 33. The data in FIGS. 33A, 33B, and 33C show that inoculation of a susceptible barley cultivar with F. graminearum significantly reduced the height of the plants, average numbers of spikes formed per plant, and the average weight of 5 spikes. However, inoculation of the barley cultivar with S. mycoparasitica did not affect growth and development. Treatment of F. graminearum-infected barley with S. mycoparasitica at an inoculation level of 10⁶ CFU/mL prevented the onset of any Fusarium head blight symptoms. Treatment of F. graminearum-infected barley with lower concentrations of S. mycoparasitica, i.e., 10⁴ CFU/mL and 10⁵ CFU/mL, did not protect against the occurrence of the disease symptoms (FIGS. 33A-33C).

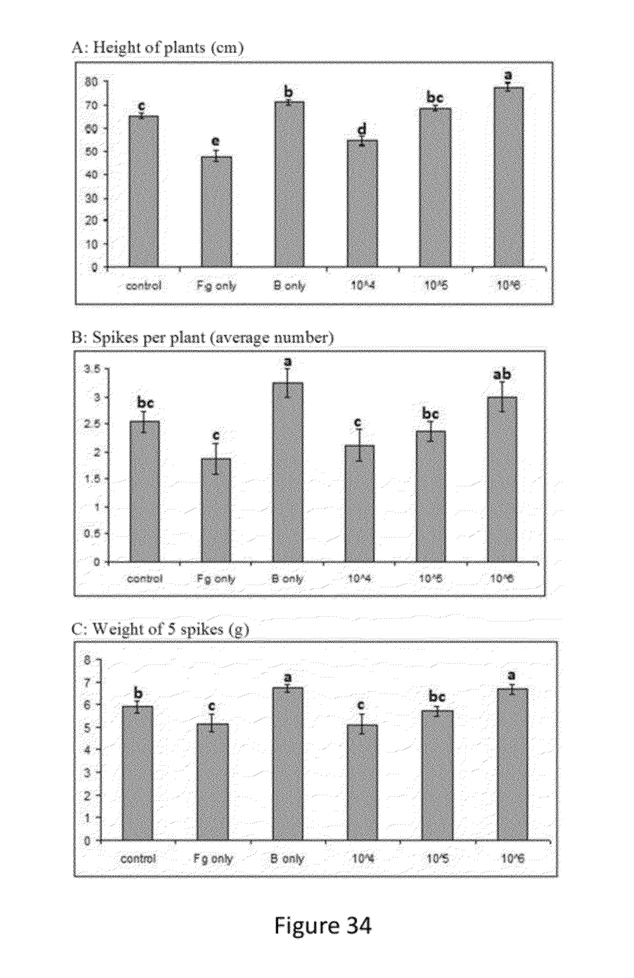

The biocontrol effects of different concentrations of S. mycoparasitica of Fusarium head blight symptoms in wheat are shown in FIG. 34. The data in FIGS. 34A, 34B, and 34C show that inoculation of a susceptible wheat cultivar with F. graminearum significantly reduced the height of the plants, average numbers of spikes formed per plant, and the average weight of 5 spikes. However, inoculation of the wheat cultivar with S. mycoparasitica did not affect growth and development. Treatment of F. graminearum-infected wheat with S. mycoparasitica at an inoculation level of 10⁶ CFU/mL prevented the onset of any Fusarium head blight symptoms. Treatment of F. graminearum-infected wheat with lower concentrations of S. mycoparasitica, i.e., 10⁴ CFU/mL and 10⁵ CFU/mL, did not protect against the occurrence of the disease symptoms (FIGS. 34A-34C).

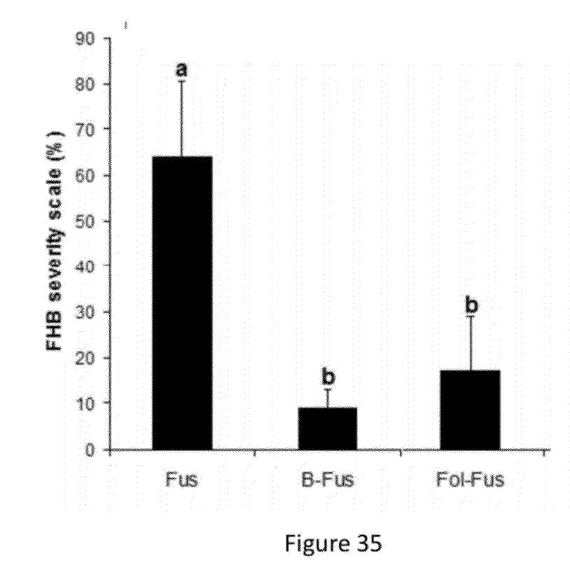

The data in FIG. 35 and Table 8 show that treatment of F. graminearum-infected barley with S. mycoparasitica at an inoculation level of 10⁶ CFU/mL provided a comparable level of protection against the occurrence of Fusarium head blight symptoms, as was provided by the commercial fungicide Folicur®.

TABLE 8
Effect of S. mycoparasitica, Folicur ® fungicide and F. graminearum on percentage of infected
spikelets, Fusarium head blight index, percentage of Fusarium damaged kernels, and weight of
100 seeds of the barley cultivar BOLD in greenhouse trials.

| | Infected spikelets | Fusarium head blight | Fusarium damaged | |
|---|---|---|---|---|
| Treatment | (%) | index (%) | kernels (%) | Weight of 100 seeds (g) |
| F. graminearum (Fg) | 62.6 ± 8.0 ^{a} | 56.7 ± 11.9 ^{a} | 75 ± 9.4 ^{a} | 4.1 ± 0.4 ^{c} |
| S. mycoparasitica-Fg | 15.4 ± 3.1 ^{bc} | 4.1 ± 1.0 ^{b} | 19 ± 6.5 ^{b} | 5.4 ± 0.6 ^{ab} |
| Folicur ®- F.g | 19.6 ± 6.9 ^{b} | 7.2 ± 4.8 ^{b} | 16 ± 6.5 ^{b} | 5.7 ± 0.5 ^{a} |

Means within each column followed by the same letter in superscript are not significantly different at P ≦ 0.05 after Kruskall-Wallace test.

Real-time PCR quantification: Total DNA was extracted from the wheat and barley spikes with DNeasy® Plant Mini Kit (Qiagen Inc., Mississauga, ON, CA). Extracted total DNAs from spikes of different treatments were employed in real-time PCR quantification. Two different primer sets and PCR conditions used in this study were described in Nicholson et al. (1998) for Fg16NF/R primer set and Schnerr et al. (2001) for Tox5-1/2 primer set. Total DNA extracted from spring wheat and barley spikes harvested from greenhouse trials were carried out in a MiniOpticon (Bio-Rad Laboratories Inc., Mississauga, ON, CA). All real-time PCR reactions were performed using real-time PCR MJ white tubes (Bio-Rad Laboratories Inc., Mississauga, ON, CA) with a total volume of 25 μl of IQ supermix (Bio-Rad Laboratories Inc., Mississauga, ON, CA), 1 μl of each 10 μM forward/reverse primers (Invitrogen), 3.4 μl of BSA (Bovine Serum Albumin) (1.47 μg/μl) (Ishii and Loynachan 2004), 5.1 μl of sterilized UltraPure Millipore water, and 2 μl of DNA template.

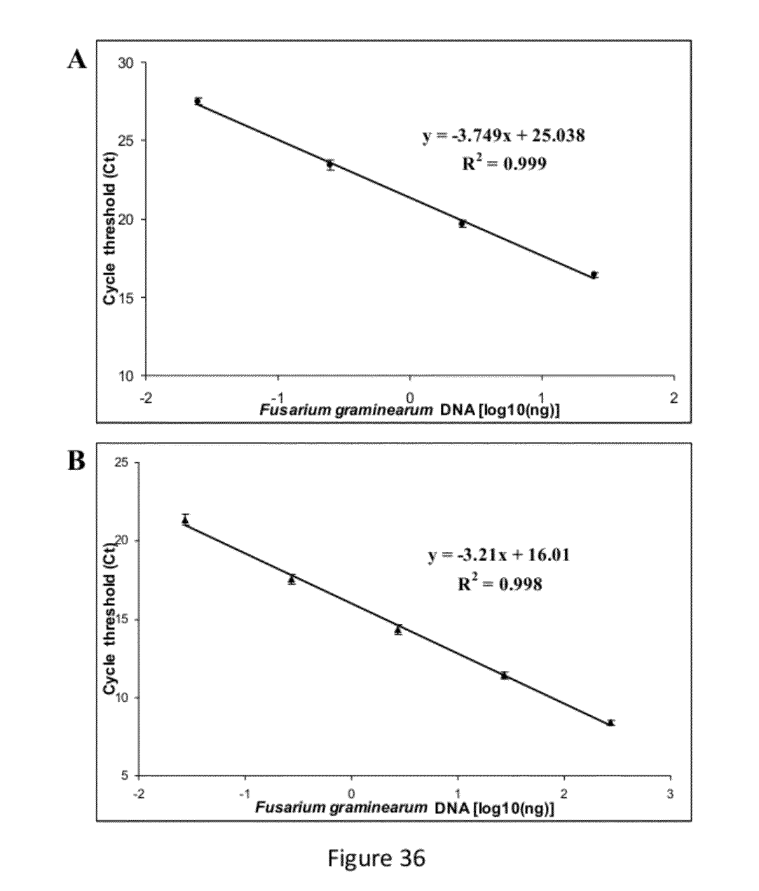

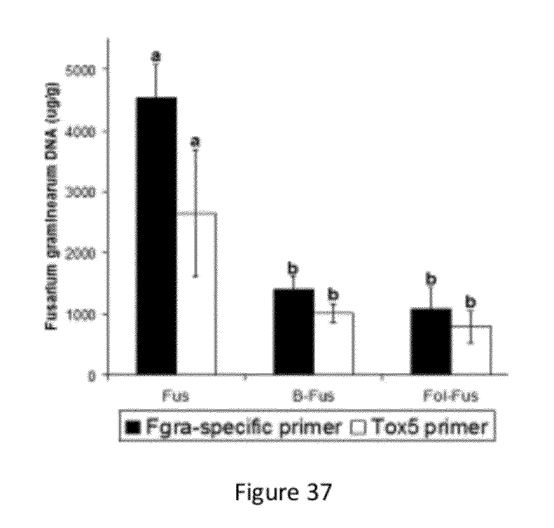

FIG. 36 shows standard curves of F. graminearum chemotype 3-ADON genomic DNA concentration standards versus cycle threshold (Ct) with PCR reactions performed in triplicate using primer sets (A) Tox5-1/2, with genomic DNA ranging from 270 ng (log₁₀=2.90) to 0.27 ng (log₁₀=−0.60), readings at 0.005 fluorescence line and (B) Fg16NF/R, with DNA template ranging from 270 ng (log₁₀=2.43) to 0.027 ng (log₁₀=−1.57); in 10-fold dilution series, readings at 0.025 fluorescence line. Error bars indicate standard deviation for the mean of F. graminearum chemotype 3-ADON standard curves derived from tri5 gene and F. graminearum specific primer sets. FIG. 37 shows effects of S. mycoparasitica (B) and Folicur fungicide (Fol) treatments on F. graminearum chemotype 3-ADON genomic DNA detected in barley spikes employing RT-PCR. Treatments were: Fus—F. graminearum; B-Fus—S. mycoparasitica with F. graminearum; Fol-Fus—Folicur fungicide with F. graminearum. All values obtained were the means of six replicates. Error bars indicate standard deviation of the mean. Means of F. graminearum DNA with Tox 5 primer were log₁₀ transformed prior to LSD test. Both primer sets were analyzed separately. Values followed by the same letters within each primer set are not significantly different using LSD test at P<0.05.

### Example 12

In addition to the known Fusarium spp. hosts for S. mycoparasitica (i.e. F. avenaceum, F. graminearum and F. oxysporum), F. culmorum and F. equiseti are also mycoparasitised by S. mycoparasitica as disclosed in the following study.

Fungal isolates and growth: Biotrophic mycoparasites S. mycoparasitica SMCD2220-01, Sphaerodes quadrangularis strain CBS112764, Sphaerodes retispora var. retispora strain CBS 994.72, and pathogenic Fusarium strains (F. arthrosporioides SMCD2247, F. culmorum SMCD2248, F. equiseti SMCD2134, F. flocciferum SMCD2135, F. poae SMCD2136, and F. torulosum SMCD2139) were obtained from the Saskatchewan Microbial Collection and Database (SMCD), Saskatchewan, Canada. All fungal isolates were grown and maintained on potato dextrose agar (PDA, BD Biosciences, Mississauga, ON, CA) prior to the study.
Fungal-fungal interactions: For examination of the interaction between isolates of Sphaerodes and Fusarium species, both biotrophic mycoparasite and Fusarium isolates were inoculated and assessed using slide culture assays. Slides were maintained in a sterile humidity chamber and daily observations on the hyphal interactions at the meeting place (contact zone) were performed under a Carl Zeiss Axioskop 2 equipped with Carl Zeiss AxioCam ICc1 camera with 20×, 40× and 100× objectives. Formation of biotrophic mycoparasitic contact structures attaching Sphaerodes species to Fusarium hyphae were examined, recorded and compared to drawings from the literature (Jordan & Barnett, 1978, Mycologia 70: 300-312; Rakvidhyasastra & Butler, 1973, Mycologia 65: 580-593; Whaley & Barnett, 1963, Mycologia 55: 199-210). Diameters of both parasitized and non-parasitized Fusarium hyphal cells were measured under light microscopy with a 100× objective lens. Each treatment used six replicates consisting of Sphaerodes or Fusarium alone, and Sphaerodes-Fusarium co-inoculated. The experiment was repeated twice. In the slide-culture assay, Fusarium mycelia infected with Sphaerodes haustoria were stained with lactofuchsin. Stained hyphae of both Fusarium and Sphaerodes in slide-culture were then examined with a Carl Zeiss Axioskop 2 fluorescent microscope attached to a Carl Zeiss AxioCam ICc1 with 40× and 100× objectives. Slide-culture assays were also subjected to Zeiss META 510 confocal laser scanning microscopy (CLSM) analysis to observe intracellular mycoparasitism under a C-Apochromat 63×N.A.1.2 phase-contrast water immersion objective through Z-stacking mode to scan through the Fusarium hyphae with intracellular infection (CLSM with 514 nm excitation—argon and LP585 emission filters).

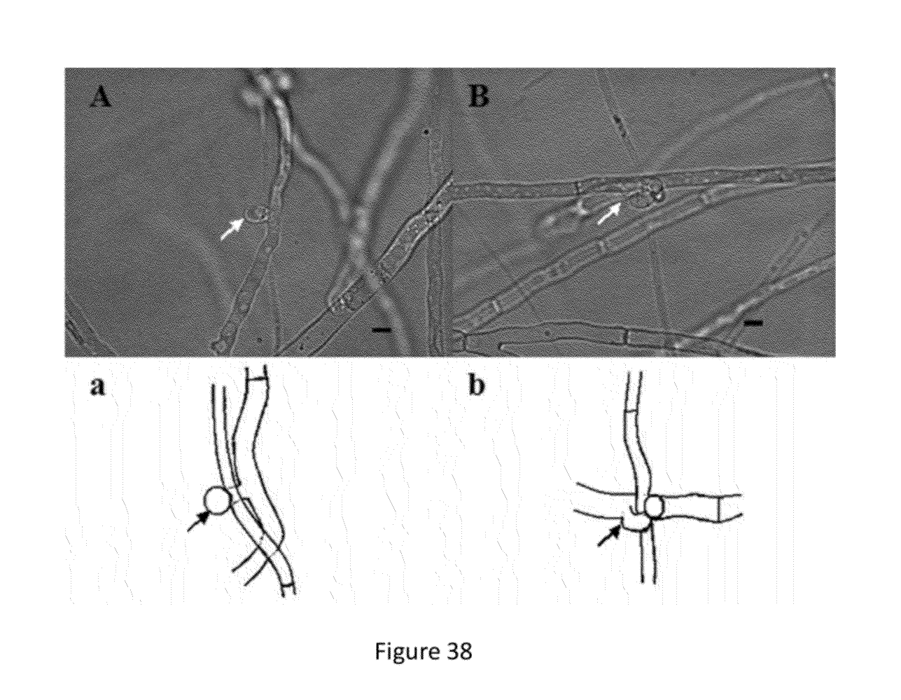

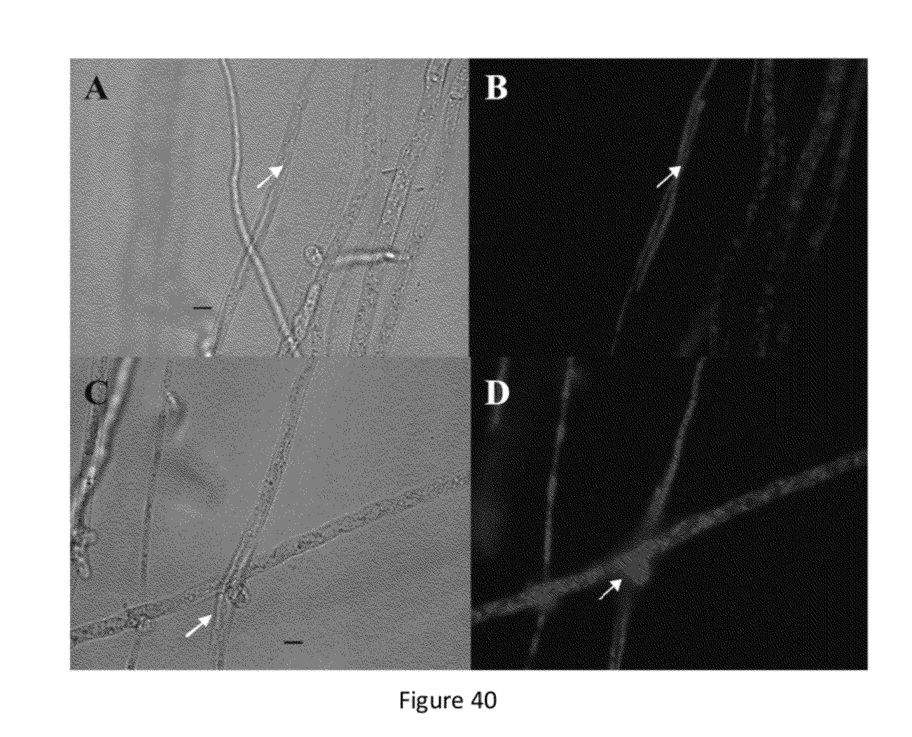

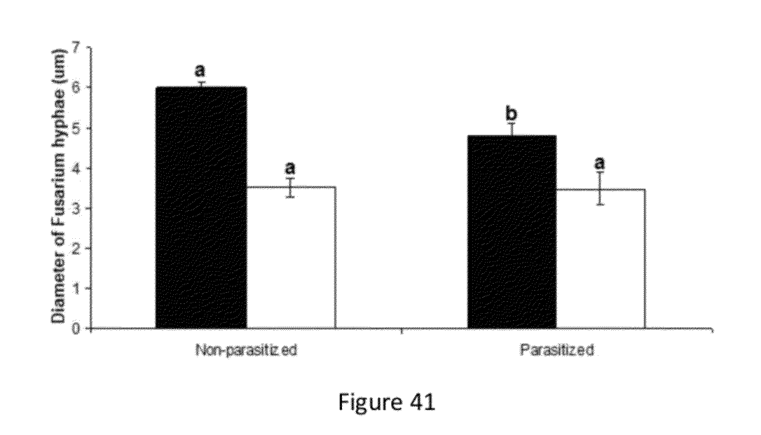

Hyphae-hyphae interactions and contact structures in the contact zone were examined for seven days. On day three, Sphaerodes mycoparasitica was found to produce hook-shaped contact structures on F. equiseti and F. culmorum (FIG. 38). On day five, more hook-shaped contact structures and intracellular penetration of F. equiseti were observed (FIGS. 38B; 39A). The combination of lactofuchsin dye and fluorescent or confocal laser scanning microscopy revealed that the parasitized or penetrated Fusarium cells became empty (loss of cytoplasm=no fluorescence) or fluoresced with low intensity (very pale) (FIGS. 40A-40B) as compared to healthy Fusarium cells. During the seven days of observation, no S. mycoparasitica hyphae were observed within F. culmorum cells. S. mycoparasitica produced hook-shaped contact structures (FIG. 38A, a) more frequently than clamp-like contact structures (FIG. 38B, b) on both F. equiseti and F. culmorum. Diameters of F. equiseti, but not F. culmorum, hyphae parasitized by S. mycoparasitica were observed to be significantly reduced compared to non-parasitized Fusarium hyphae (with T-test, P=0.001 and P>0.05, respectively) (FIG. 41).

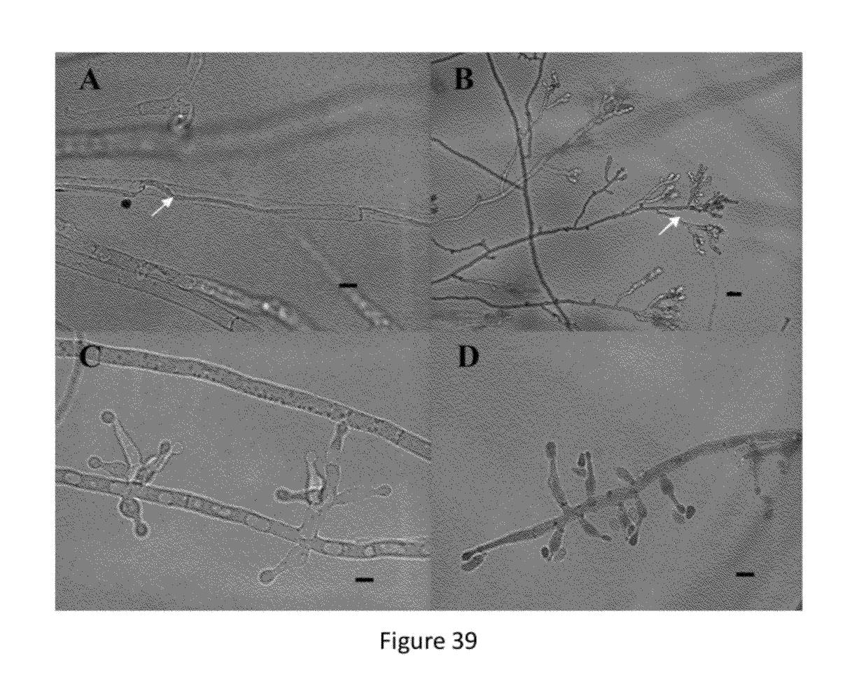

None of the Fusarium taxa tested appeared to be suitable hosts for mycoparasitic S. quadrangularis and S. retispora even after 10 days of co-inoculation on slide cultures. No contact biotrophic parasitic structures or intracellular parasitism by S. quadrangularis and S. retispora on the tested Fusarium strains were observed at the interaction or contact zone. Also, F. arthrosporioides, F. flocciferum, F. poae, and F. torulosum did not appear to be suitable hosts for S. mycoparasitica. Around five days after inoculation on slide culture assays, mycelia of F. arthrosporioides were inhibited by S. mycoparasitica. F arthrosporioides started to form rosette-like mycelia at the contact zone with S. mycoparasitica (FIG. 39B).

On the fifth and seventh days after inoculation, anamorphic structures were produced by S. mycoparasitica more abundantly in the zone of contact with F. culmorum (FIGS. 40C and 40D). Anamorphic structures or asexual organs in close proximity to F. culmorum mycelia were red-colored (FIG. 40D), whereas the organs at a distance were not (FIG. 40C).

### Example 13

### Production and Extraction of Intracellular and Extracellular Proteins from Sphaerodes mycoparasitica

Sphaerodes mycoparasitica strains IDAC 301008-02 and/or IDAC 301008-03 were used in all of the following examples, and are referred throughout as “SM-Bst” and “SMGst” respectively. Stock cultures of SM-Bst and SM-Gst were prepared and maintained in potato dextrose broth (PDB) culture media at 21° C. To prepare intracellular and extracellular proteins from each type of stock culture, about 3 ml were transferred from the PDB culture to 250-ml Erlenmeyer flasks containing 50 ml PDB growth medium. The flasks were incubated for 7 days on a rotary shaker (150 rpm) at room temperature. Young mycelia were filtered through Whatman® No. 1 filter paper (Whatman is a registered trademark of Whatman International Ltd., Kent, UK). Mycelia collected on the Whatman® No. 1 filter paper were used for extraction of intracellular proteins, whereas the filtered culture media was used for recovery of extracellular proteins therefrom.

### Example 14

### Fractionation, Separation and Purification of Intracellular Proteins and Extracellular Proteins from Sphaerodes mycoparasitica

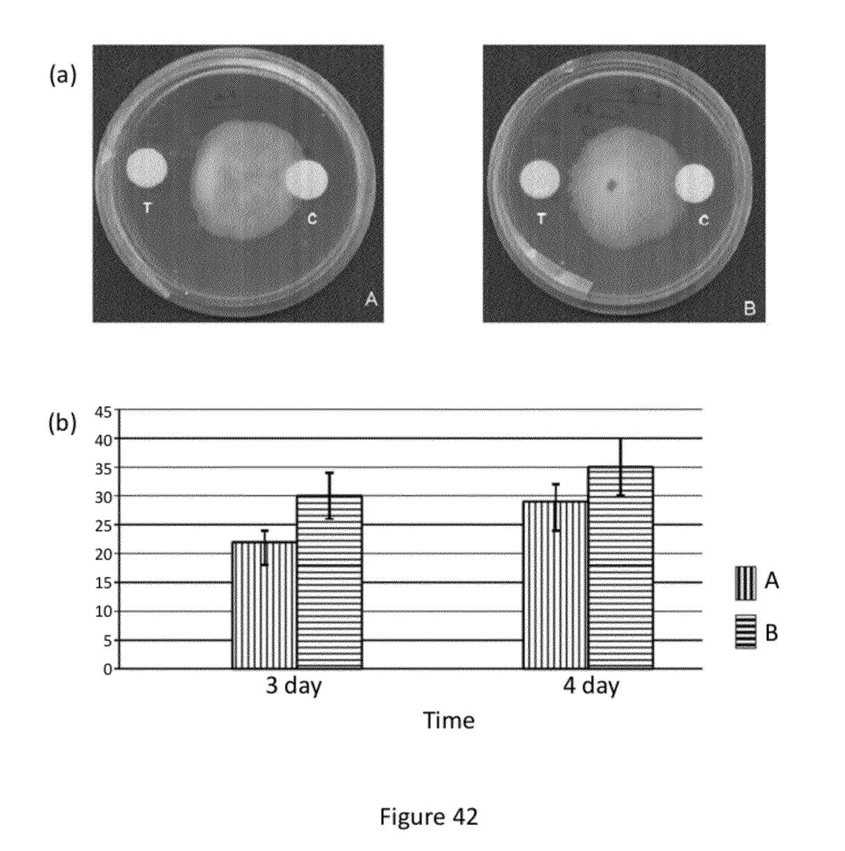

Intracellular protein extraction: Intracellular proteins were extracted according to the method disclosed by Chen et al. (2004, Heat shock proteins of thermophilic and thermotolerant fungi from Taiwan. Bot. Bull. Acad. Sin. 45: 247-257). A total of 200 mg of the fungi mycelium from each sample was powdered in liquid nitrogen in a sterile porcelain mortar using a pestle and mixed with 1.5 ml chilled extraction buffers. Mycelia (˜200 mg) were ground by sonication in a grinding tube containing 1 mL of buffer consisting of 50 mM Tris-HCl (pH 8.5); 2% sodium dodecyl sulfate (SDS); 2% β-mercaptoethanol; 1 mM phenylmethylsulfonyl fluoride (PMSF in 95% alcohol). The homogenate was centrifuged at 12,000 rpm at 28° C. for 10 min, and the supernatant was collected. Four volumes of cold acetone (−20° C.) were added, and the samples were frozen overnight at −20° C. The precipitate (proteins) was stored in acetone, centrifuged at 12000 rpm at 28° C. for 5 min, and finally dissolved in 50-100 μl of sample buffer (62.5 mM Tris-HCl (pH 6.8); 3% SDS; 10% glycerol; 5% β-mercaptoethanol). The protein samples were transferred to new 1.5 ml eppendorf tubes and stored at −80° C. until used for further studies.
Extracellular protein extraction: Filtered culture medium was used as the source of extracellular proteins and was concentrated by Amicon® ultrafiltration centrifuge tube with a 3000-Dalton cut-off membrane (Amicon is a registered trademark of the Millipore Corp., Billerica, Mass., USA) by centrifugation at 4000 rpm at 4° C. Total extracellular proteins were tested for antifungal activity against the fungal pathogens, Fusarium oxysporum and Fusarium graminearum using a disc diffusion assay. Antifungal activities of total extracellular proteins were tested under sterile conditions by radial disc plate diffusion assay as described by Roberts and Selitrennikoff (1986, Isolation and partial characterization of two antifungal proteins from barley. Biochim. Biophys. Acta, 880: 161-170) with some modifications. Testing of the total extracellular proteins for antifungal activity toward F. oxysporum and F. graminearum was carried out in petri plates containing potato dextrose agar. Mycelial plugs from actively growing fungal plates were placed in the center of the petri plates and sterile filter paper discs (5 mm diameter of Whatman® filter paper no. 1) were placed on the agar surface at a distance of 0.5 cm away from the rim of the mycelial colony. An aliquot (60 μL) containing 2.5 μg of extracellular protein was added to a disk (test spot is identified in FIGS. 42(a)A and 42(a)B as “T”). Sterile distilled water and buffer served as controls (control spot is identified in FIGS. 42(a)A and 42(a)B as “C”). The plates were then incubated at room temperature for 4 days and examined for inhibition of mycelial outgrowth. The areas of the mycelial colonies were measured and the inhibition of fungal growth was determined by calculating the percent reduction in area of mycelial colony outgrowth relative to the control. The total extracellular proteins recovered from the SMCD culture inhibited mycelial outgrowth in both F. oxysporum and F. graminearum (FIGS. 42(a)A and 42(a)B). Three days after germination commenced, about 22% inhibition in the hyphal extension of F. oxysporum and 30% inhibition in hyphal extension of F. graminearum were observed (FIG. 42(b)). One day later, inhibition of hyphal extension of F. oxysporum increased to about 30%, while inhibition of hyphal extension in F. graminearum increased to about 35% (FIG. 42b).
Native polyacrylamide gel electrophoresis (native-PAGE): Proteins (intracellular and extracellular) were separated by 10% native polyacrylamide gel electrophoresis (native-PAGE) in a discontinuous buffer system using the method taught by Laemmli (1970, Cleavage of structural protein during the assembly of the head of bacteriophage T4. Nature 227: 680-685). The stacking gel (3.75% acrylamide/bis-acrylamide 30:0.8 v/v) had 125 mM Tris-HCl, pH 6.8 as buffer and the separating gel (7.5% acrylamide/bis-acrylamide 30:0.8 v/v) had 375 mM Tris-HCl, pH 8.8 as buffer. The gels were run at 120 V overnight at 4° C. with a Tris-glycine electrode buffer containing 25 mM Tris and 192 mM glycine, pH 8.3. After electrophoresis, the gel was stained according to a silver staining method (Bio-Rad® kit methods).
Sodium dodecyl sulfate polyacrylamide gel electrophoresis (SDS-PAGE): SDS-PAGE of proteins eluted from native-PAGE and total proteins was performed on 12% polyacrylamide gel following the method taught by Laemmli (1970). Proteins were analyzed by using 5% stacking gel (pH 6.8) and 12% separating acrylamide gel (pH 8.8) in Tris-glycine buffer (pH 8.3) and appropriate markers. Prior to SDS-electrophoresis, the protein was mixed with an equal volume of sample buffer (60 mM Tris-HCl buffer, 4% SDS, pH 6.8) containing 5% β-mercaptoethanol. A mixture of standard marker proteins obtained from Bio-Rad® Laboratories Canada Ltd. (Montreal, PQ, Canada; Bio-Rad is a registered trademark of Bio-Rad Laboratories Inc., Hercules, Calif., USA) was used. All samples were heated for 5 min at 95° C. and cooled to room temperature. Proteins were visualized by silver staining method (Bio-Rad® kit methods). Protein molecular masses were estimated by comparison with the mobilities of standard molecular mass markers.
Fast protein liquid chromatography (FPLC) of extracellular proteins: Proteins were fractionated through Superdex® 75 GL 10/30 column (Superdex is a registered trademark of GE Healthcare Bio-Sciences AB Ltd., Uppsala, Sweden) using FPLC AKTA® purifier system (AKTA is a registered trademark of GE Healthcare Bio-Sciences AB Ltd., Uppsala, Sweden) according to the manufacturer's instructions. The column was previously equilibrated with sterile water and with 50 mM sodium phosphate buffer, pH 7.0 containing 0.15 M NaCl, followed by protein injection (about 500 μL) and elution of proteins with the same buffer with a flow rate of 1.0 ml/min. Proteins were purified through gel filtration using 50 mM sodium phosphate buffer, pH 7.0 containing 0.15 M NaCl. Upon gel filtration on Superdex 75, proteins were resolved into distinct peaks. 0.8-ml fractions were collected and their purities were checked by SDS-PAGE.
Sodium dodecyl sulfate-polyacrylamide gel electrophoresis (SDS-PAGE): SDS-PAGE (12%) of proteins recovered from FPLC fractions was performed following the method taught by Laemmli (1970). All peaks giving FPLC fractions were pooled and recovered by precipitation in 1:4 volume of chilled acetone and kept at −20° C. overnight. After centrifugation at 12,000 g for 10 min, precipitated proteins (pellets) were dissolved in minimum amount (30 μL) of assay buffer. Proteins were analyzed by SDS-PAGE having 5% stacking gel (pH 6.8) and 12% separating acrylamide gel (pH 8.8) in Tris-glycine buffer (pH 8.3) and appropriate markers. Prior to SDS-electrophoresis, the protein was mixed with an equal volume of sample buffer (60 mM Tris-HCl buffer, 4% SDS, pH 6.8) containing 5% β-mercaptoethanol. A mixture of standard marker proteins (Bio-Rad® protein markers, Bio-Rad Laboratories Canada Inc.) was used. All samples were heated for 5 min at 95° C. and cooled to room temperature before loading on gel. Proteins were visualized by silver staining method (Bio-Rad® Silver staining kit, Bio-Rad Laboratories Canada Inc.). Proteins molecular masses were estimated by comparison with the mobilities of standard molecular mass markers. The molecular masses of the fractionated proteins obtained from FPLC were determined by SDS-PAGE (12% and 16%). 5% SDS-PAGE was performed to separate the extracellular proteins comprising the large weight protein bands separated by the native-PAGE process.

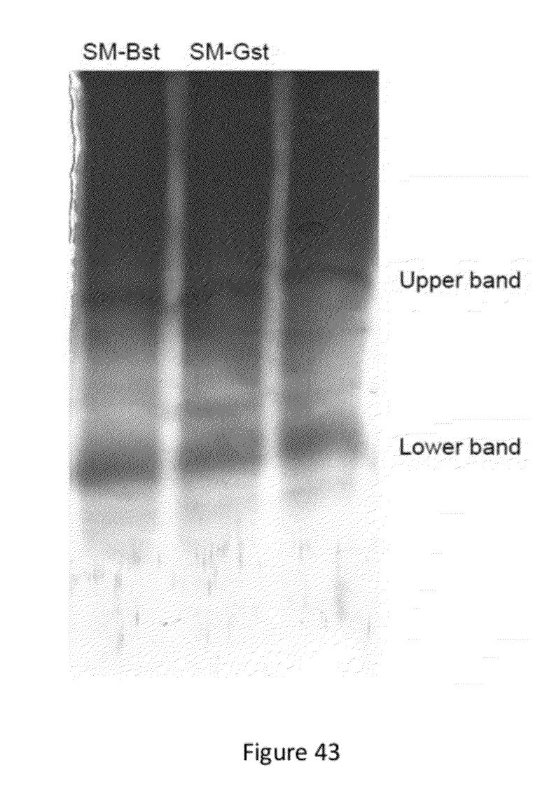

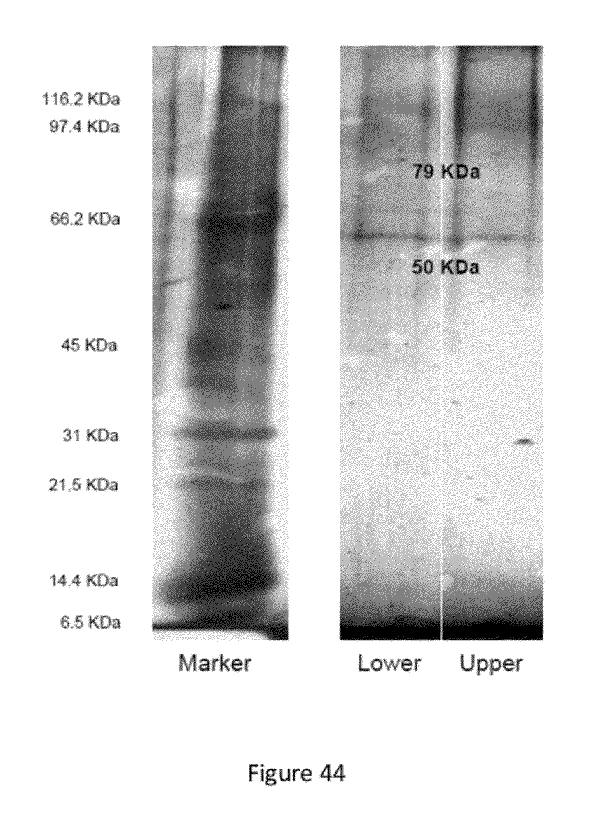

Native-PAGE electrophoresis of intracellular proteins extracted from SM-Bst and SMGst using 10% native polyacrylamide gels separated two bands of proteins designated as upper bands and lower bands (FIG. 43). Each protein band of native-PAGE was immersed in 500-750 μl of sample buffer (62.5 mM Tris-HCl, pH 6.8; 1% PMSF, 1% EDTA) and left overnight at 4° C. The proteins were recovered by centrifugation at 12,000 g for 10 min. SDS-PAGE separation of the intracellular proteins was performed using 12% gels for determination of the molecular weights of the separated proteins eluted from native-PAGE. The eluted proteins from lower and upper bands of intracellular samples showed presence of two distinct bands of approximately 50 and 79 kDa (FIG. 44).

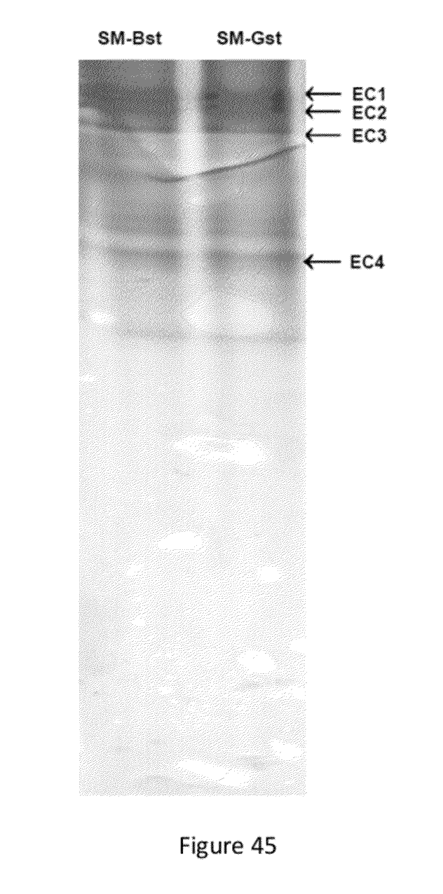

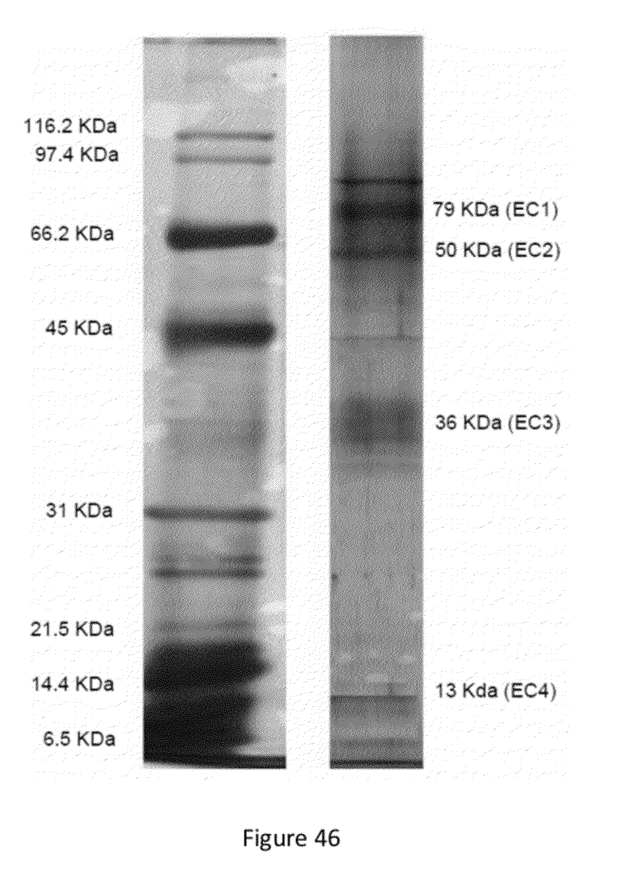

Native-PAGE electrophoresis of extracellular proteins extracted from SM-Bst and SMGst using 10% native polyacrylamide gels separated four bands of proteins designated as EC1, EC2, EC3, and EC4 (FIG. 45). Each protein band of native-PAGE was immersed in 500-750 μl of sample buffer (62.5 mM Tris-HCl, pH 6.8; 1% PMSF, 1% EDTA) and left overnight at 4° C. The proteins were recovered by centrifugation at 12,000 g for 10 min. SDS-PAGE separation of the extracellular proteins was done using 12% gels for determination of the molecular weights of the separated proteins eluted from native-PAGE. The eluted proteins from the four bands showed presence of separated and purified proteins having molecular weights of about: (i) 79 kDa (EC1), (ii) 50 kDa (CE2), (iii) 36 kDa (EC3), and 13 kDa (EC4) (FIG. 46).

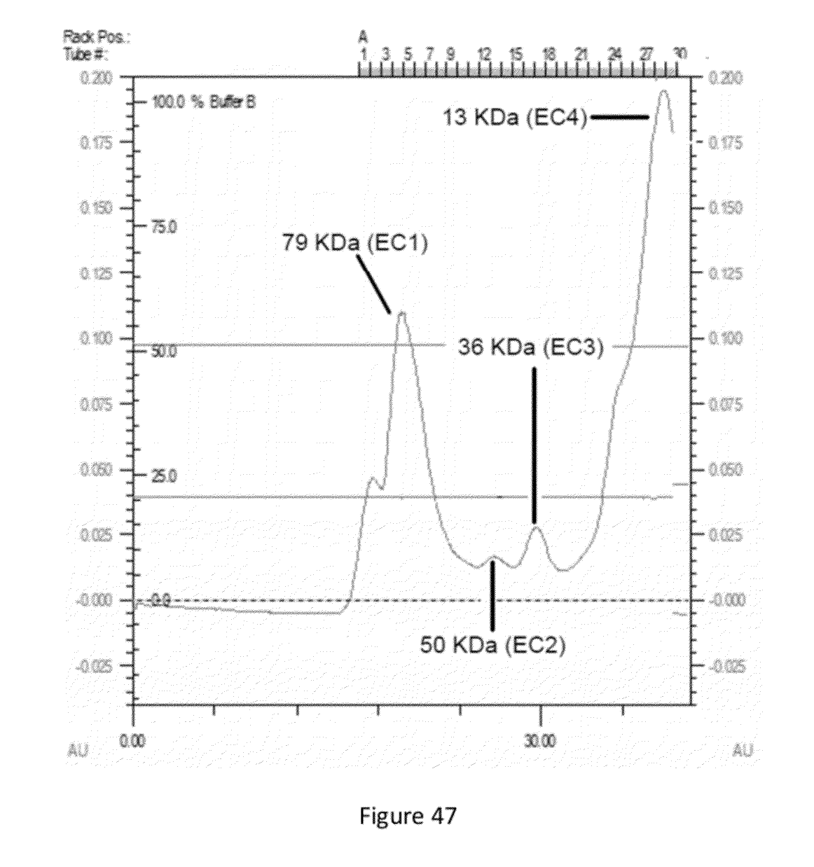

The elution profile of the four bands of extracellular proteins on a Superdex 75 GL 10/30 column by determined by FPLC fast protein liquid chromatography using an AKTA® Purifier System is shown in FIG. 47.

### Example 15

### Inhibition of Mycelial Outgrowth by Fusarium sp. by Purified Intracellular Proteins and Purified Extracellular Proteins Recovered from S. mycoparasitica Cultures

Antifungal activities of proteins were tested under sterile conditions by radial disc plate diffusion assay following the method taught by Roberts et al. (1986, Isolation and partial characterization of two antifungal proteins from barley. Biochim. Biophys. Acta, 880: 161-170) with some modifications. The assay of the isolated protein for antifungal activity toward F. oxysporum and F. graminearum was carried out in petri plates containing potato dextrose agar. Mycelial plugs from actively growing fungal plates were placed in the center of the petri plates and sterile filter paper discs (5-mm diameter of Whatman® filter paper no. 1) were placed on the agar surface at a distance of 0.5 cm away from the rim of the mycelial colony. Isolated proteins (60 μL) were added to the disc. Sterile distilled water and buffer only without antifungal protein served as a control. The plates were then incubated at room temperature for 3-4 days and then examined for zones of inhibition, if any, around the discs. In this manner, if the protein being tested was an antifungal agent, a crescent-shaped zone of inhibition of fungal growth would have occurred around the disc. The area of the mycelial colony was measured and the inhibition of fungal growth was determined by calculating the % reduction in area of mycelial colony with the controls (water and buffer treated plates).

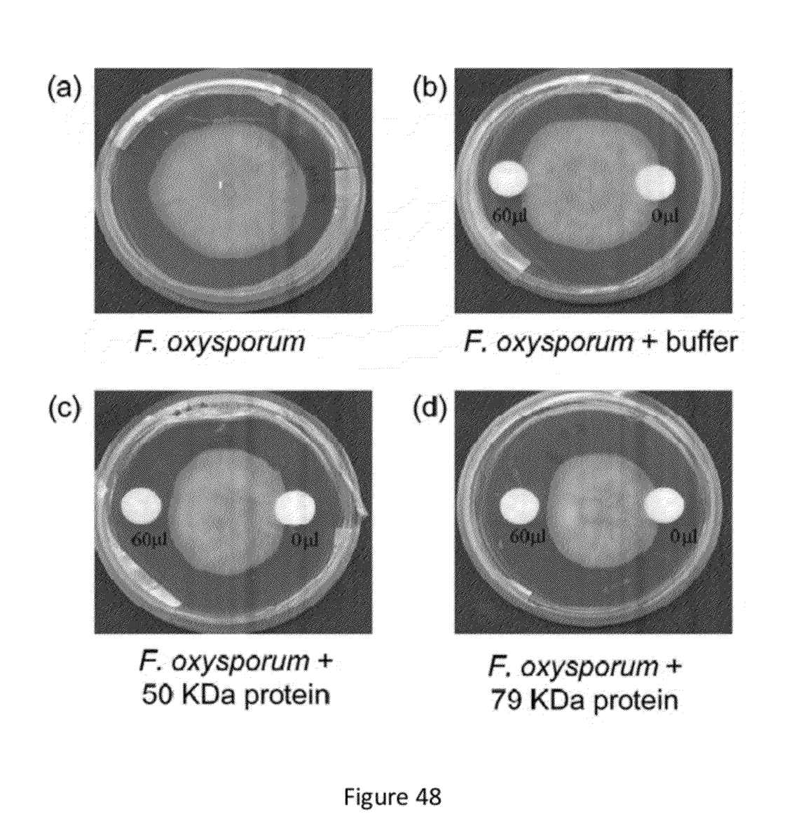

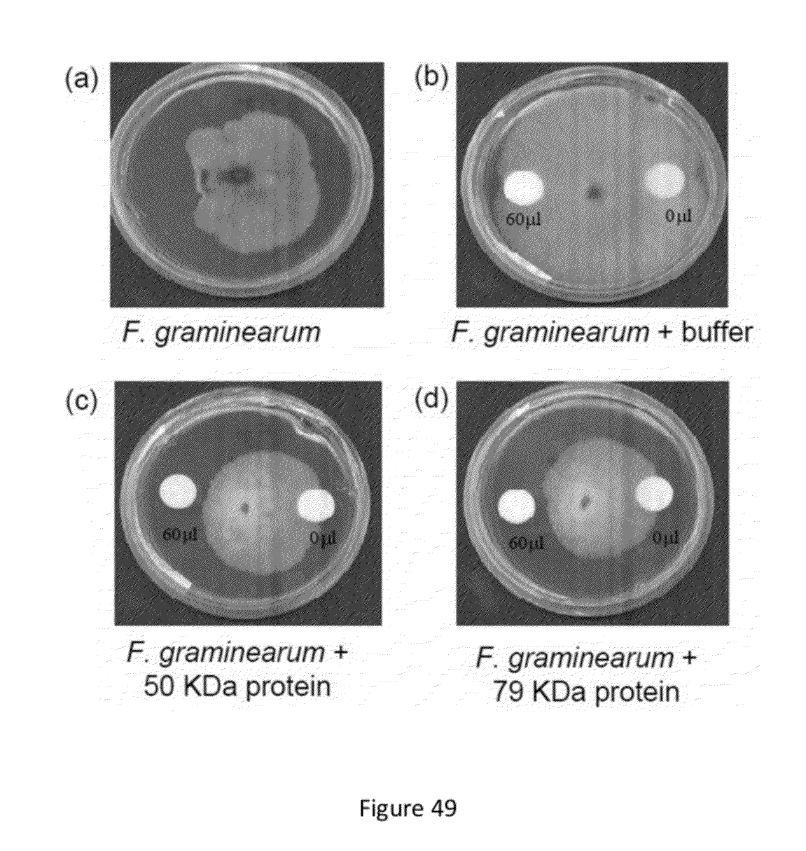

Both the 50 kDa intracellular protein and the 79 kDa intracellular protein inhibited the hyphal extension, i.e., mycelial growth of F. oxysporum (FIGS. 48(a)-48(d)) and F. graminearum (FIGS. 49(a)-49(d)).

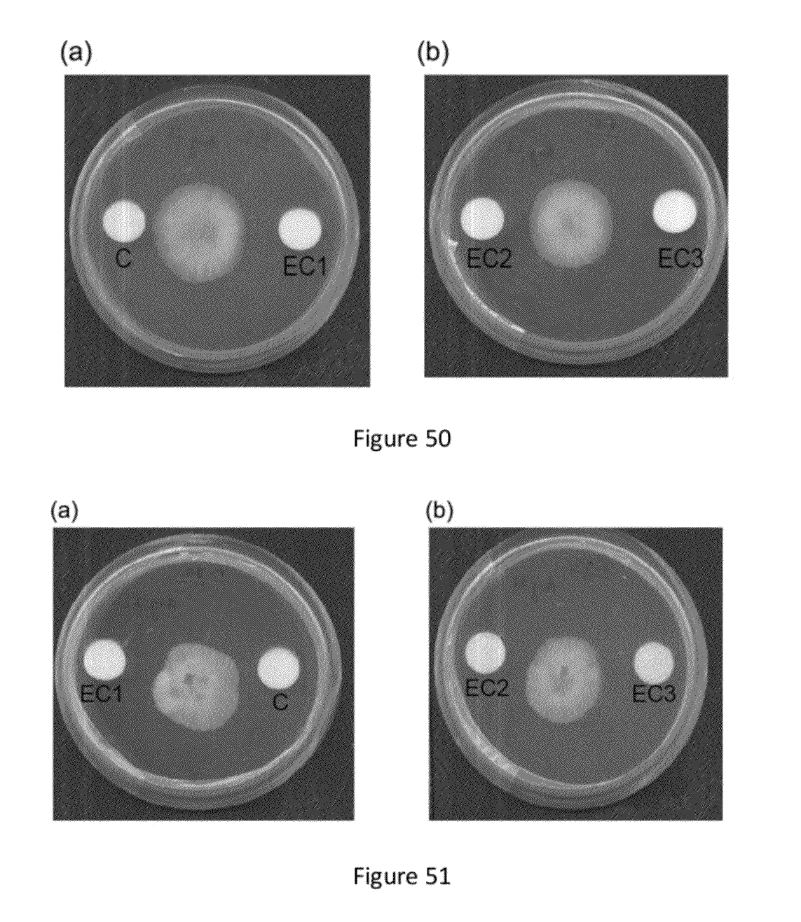

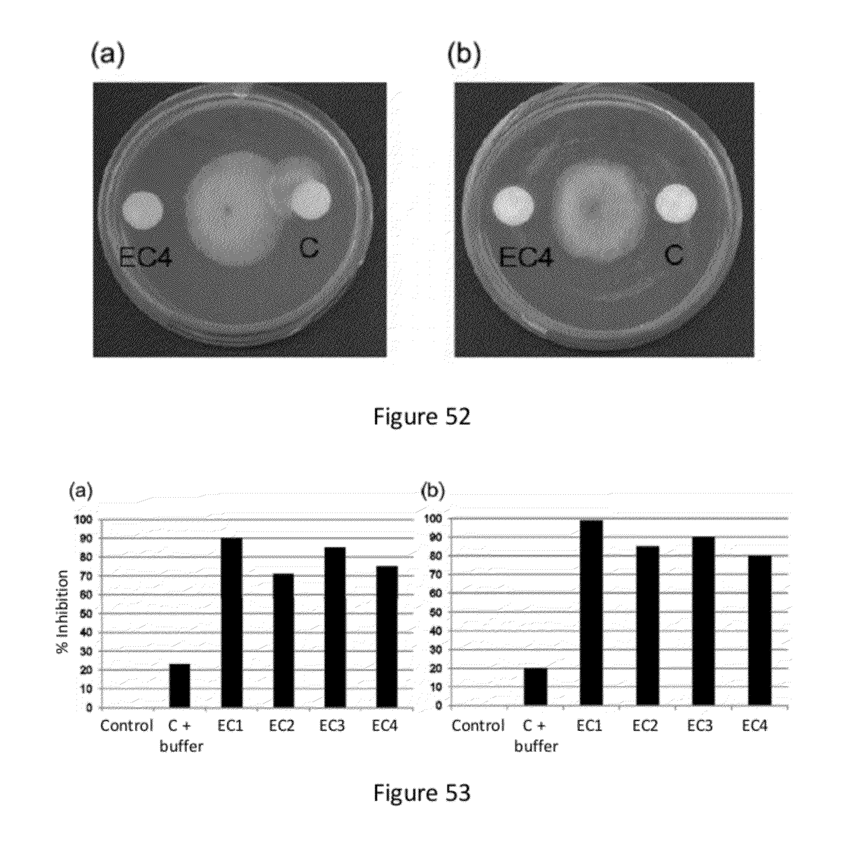

The 79 kDa protein (EC1), the 50 kDa protein (EC2), and the 36 kDa protein (EC3) inhibited mycelial outgrowth of F. oxysporum (FIGS. 50(a)-50(b)) and F. graminearum (FIGS. 51(a)-51(b)). The 13 kDa protein (EC4) inhibited mycelial outgrowth of F. graminearum (FIG. 52(a)) and F. oxysporum (FIG. 52(b)).

### Example 16

### Inhibition of Spore Germination by Fusarium sp. by Purified Extracellular Proteins Recovered from S. mycoparasitica Cultures

The inhibitory effects on Fusarium sp. spore germination by the four extracellular S. mycoparasitica proteins EC1, EC2, EC3, and EC4 were assessed using the microtitre plate assay method disclosed by Ghosh (2006, Antifungal Properties of Haem Peroxidase from Acorus calamus. Ann. Bot. 98: 1145-1153) and Yadav et al. (2007, An antifungal protein from Escherichia coli. J. Med. Microbiol. 56: 637-644). The possible toxicity of the fractionated proteins was tested using the fungi F. oxysporum and F. graminearum with a percentage growth inhibition assay. The in vitro antifungal activities of fractionated proteins were determined in 96-well microtiter plates. In microplate wells, 10 μl of potato dextrose broth (PDB; Difco Laboratories, Detroit) were mixed with 3 μl of spore suspensions of F. oxysporum and F. graminearum. An aliquot of 7 μL of different peak containing proteins fractions were added to suspensions in microtitre plates (12-8 wells). Water and buffer were used as negative controls. The microtiter plate was then incubated at room temperature in dark. Observations were made for inhibition of spore germination in both untreated and treated wells after 24 h using inverted and fluorescent microscopes. The number of germinated and non-germinated spores and percentage of area covered by mycelia in microscope were used to determine the percentage of growth inhibition.

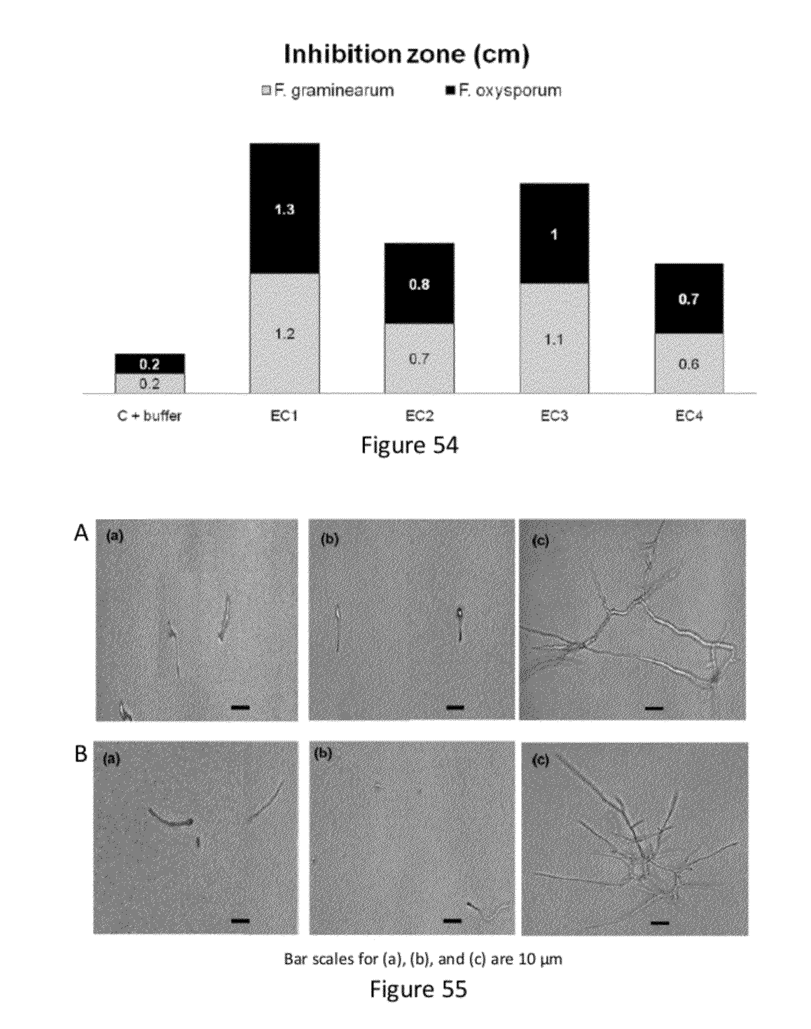

All four extracellular proteins significantly inhibited germination of the spores of F. oxysporum and F. graminearum (FIGS. 53(a) and 53(b)). Furthermore, all four extracellular proteins established large zones of inhibition of mycelial growth of F. oxysporum and F. graminearum when these organisms were separately plated onto solid media (FIG. 54). FIG. 55A(c) is a micrograph showing germination of control F. oxysporum spores, FIG. 55A(a) shows the inhibitory effects of the 50 kDa protein on germination of F. oxysporum spores, and FIG. 55A(b) shows the inhibitory effects of the 13 kDa protein on germination of F. oxysporum spores. FIG. 55B(c) is a micrograph showing germination of control F. graminearum spores, FIG. 55B(a) shows the inhibitory effects of the 50 kDa protein on germination of F. graminearum spores, and FIG. 55(b) shows the inhibitory effects of the 13 kDa protein on germination of F. graminearum spores.

### Example 17

### Inhibitory Effects of Crystal-Forming EC1 and EC3 Proteins Recovered from S. mycoparasitica Cultures

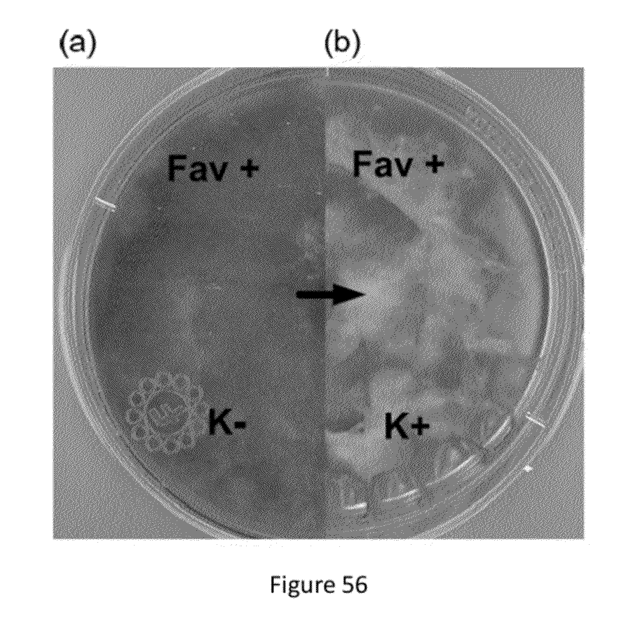

The mycotoxic effects of crystal-forming extracellular proteins EC1 (79 KDa) and EC3 (36 KDa) were assessed with actively growing F. avenaceum and F. graminearum cultures on PDA. F. avenaceum was grown on PDA agar until the mycelia covered the surface of the plate. One half of the plate was treated with a mixture of EC1 and EC3 crystal proteins, after which the plate was incubated in the dark at 23° C. for 5 days. Significant mycelial damage was observed on the half of the plate that was treated with the EC1/EC3 protein mixture (FIG. 56(b)) compared to the untreated control side of the plate (FIG. 56(a)).

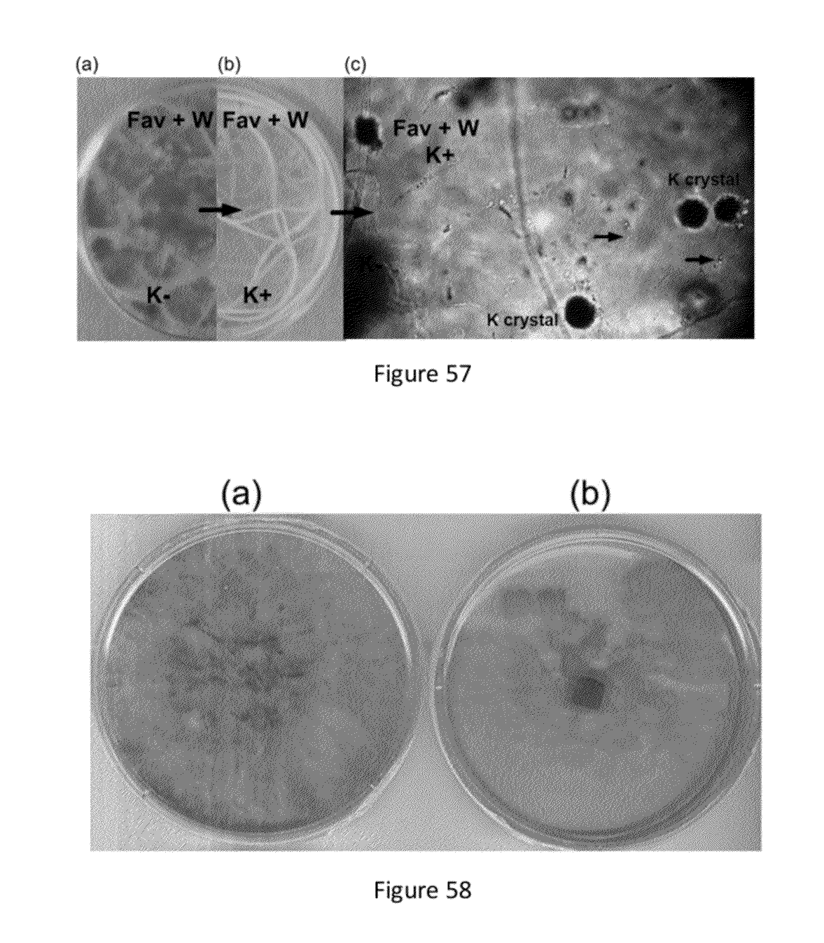

Wheat seeds were sown across the surface of a PDA plate. After the seeds had germinated, the plates were inoculated with F. graminearum. One half of the plate was treated with the EC1/EC3 protein mixture, after which the plate was incubated in the dark at 23° C. for 5 days. There was no fungal growth on the half of the plate that received the EC1/EC3 protein mixture (FIG. 57(b)) while fungal mycelia proliferated on the untreated side of the plate (FIG. 57 (a)). Examination of the surfaces of the agar that had received the EC1/EC3 protein mixture, with confocal laser scanning microscopy, showed numerous lysed hyphal elements (see arrows in FIG. 57(c)).

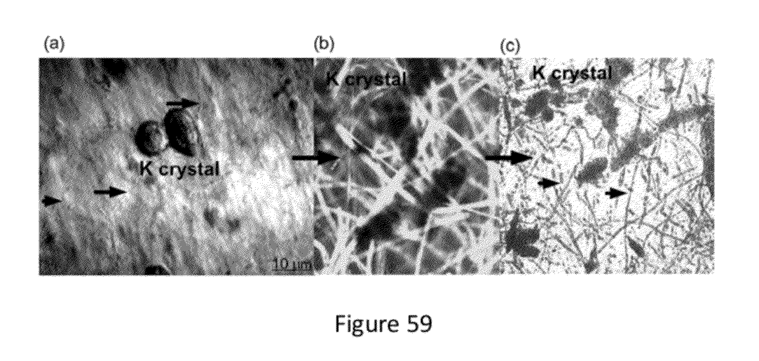

F. graminearum was grown on PDA agar until the mycelia covered the surface of the plate. One half of the plate was treated with a mixture of EC1 and EC3 crystal proteins after which, the plate was incubated in the dark at 21° C. for 5 days. Significant mycelial damage was observed on the half of the plate that was treated with the EC1/EC3 protein mixture (FIG. 58(b)) compared to the untreated control side of the plate (FIG. 58(a)). Examination of the surfaces of the agar that had received the EC1/EC3 protein mixture, with scanning electron microscopy showed numerous lysed hyphal elements (see arrows in FIG. 59(a)). Examination of the same surfaces with chemical force microscopy showed numerous broken hyphal elements and apoptotic-lysed cells (see arrows in FIG. 59(c)).

### Example 18

### Sequencing and Putative Identification of Purified Extracellular Proteins Recovered from S. mycoparasitica Cultures

The extracellular protein bands EC1 (79 Kda), EC2 (50 Kda), EC3 (36 Kda), and EC4 (13 KDa) were cut from 5%, 12% and 16% SDS-PAGE gels. The excised protein bands were sent to the McGill University and Genome Quebec Innovation Centre (Rm 704, 740 Dr. Penfield Avenue, Montréal, PQ, Canada) for sequencing and peptide characterization using MALDI-TOF-MS equipment, and putative identification by comparing the peptide sequences to the UniProtKB/Swiss-Prot and NCBI databases.

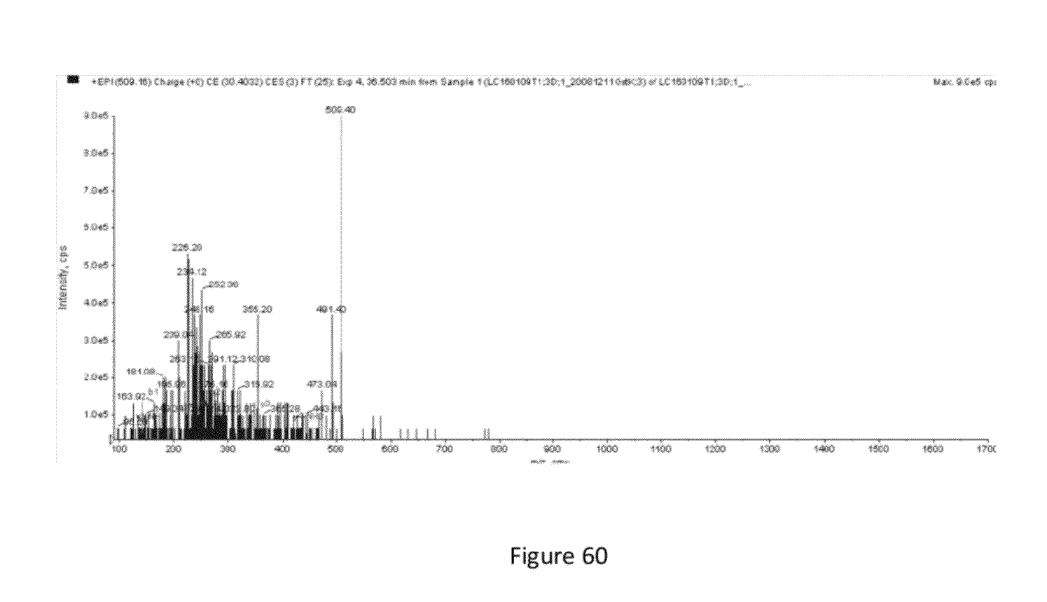

MALDI-TOF-MS analysis enabled separation of the amino acids of the EC1 protein (FIG. 60). The sequence of amino acids of the EC1 protein was determined to be:

(SEQ ID NO: 36)
YLPGGGGGRDEPPPR

The mass of EC1 was determined to be 78,688 Da (79 kDa), and its identity was predicted to be similar to the RTX protein toxins and related Ca²⁺⁻binding proteins.

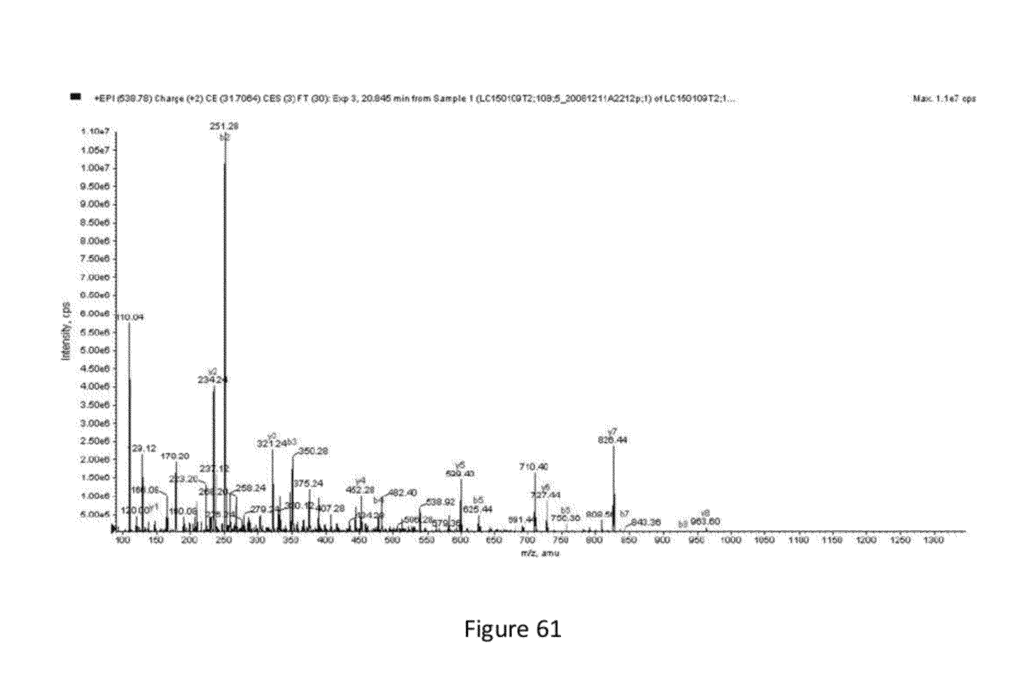

MALDI-TOF-MS separation of the EC2 extracellular protein band (FIG. 61) enabled determination of the amino acid sequence of the EC2 protein:

(SEQ ID NO: 37)
LHVQFMSSK

The mass of EC2 was determined to be 49,443 Da (50 kDa), and its identity was predicted to be similar to β-1,4-glucase proteins.

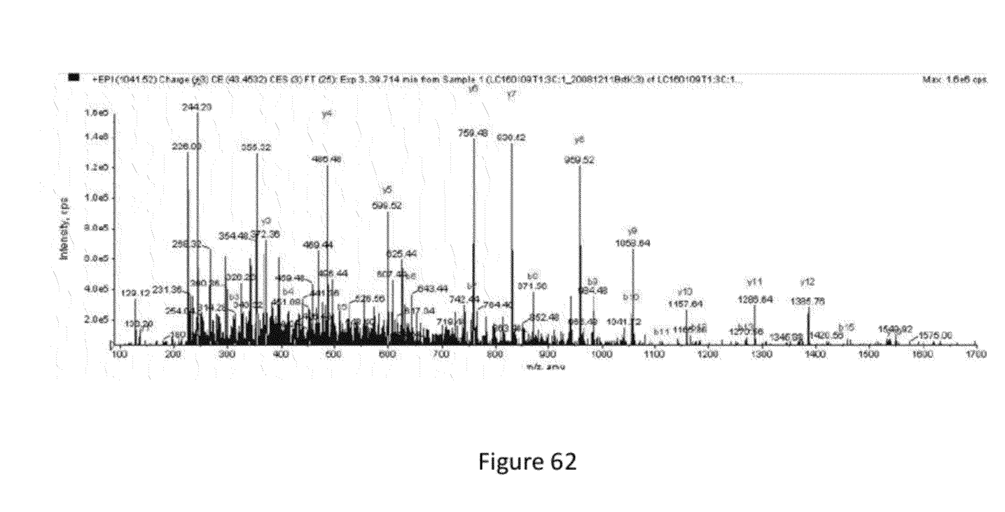

MALDI-TOF-MS separation of the EC3 extracellular protein band (FIG. 62) enabled determination of the amino acid sequence of the EC3 protein:

(SEQ ID NO: 38)
EIAVTELDIAGASSTDYVEVVEACLNQPK

The mass of EC3 was determined to be 35,578 Da (36 kDa), and its identity was predicted to be similar to Xylanase proteins.

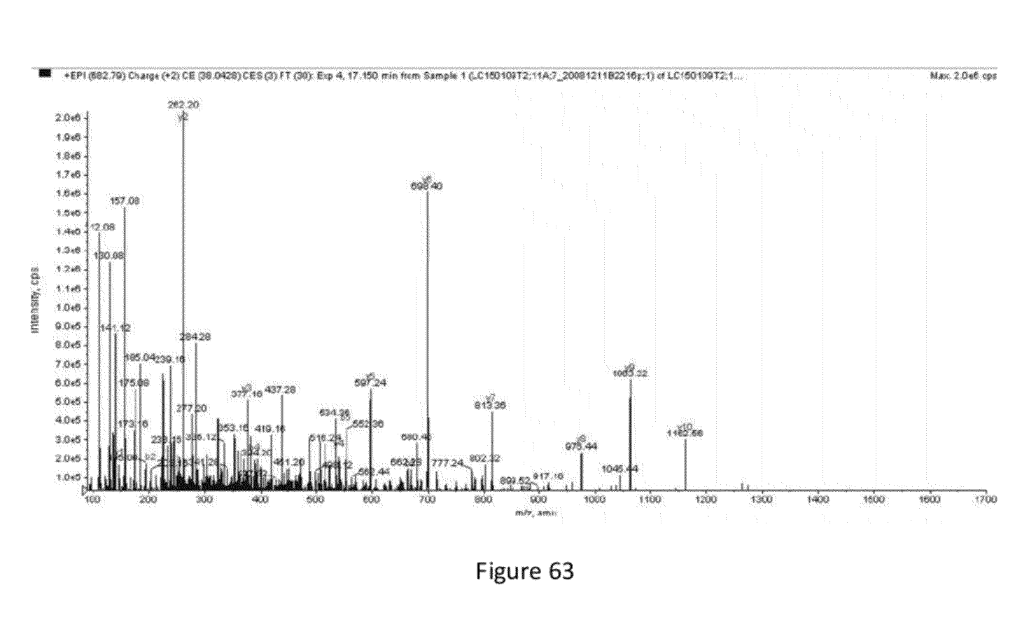

MALDI-TOF-MS separation of the EC4 extracellular protein band (FIG. 63) enabled determination of the amino acid sequence of the EC4 protein:

(SEQ ID NO: 39)
TTVSYDTGYDDK

The mass of EC4 was determined to be 2,563 Da (this is a truncated fragment because the SDS-Page analysis showed its molecular weight to be 13 kDa), and its identity was predicted to be a hypothetical Cerato-platanin toxin.

### Example 19

### Effects of Purified Extracellular Proteins Recovered from S. mycoparasitica Cultures on mycelial growth and development of Sclerotinia sclerotiorum, Rhizoctonia solani, and Pythium ultimum

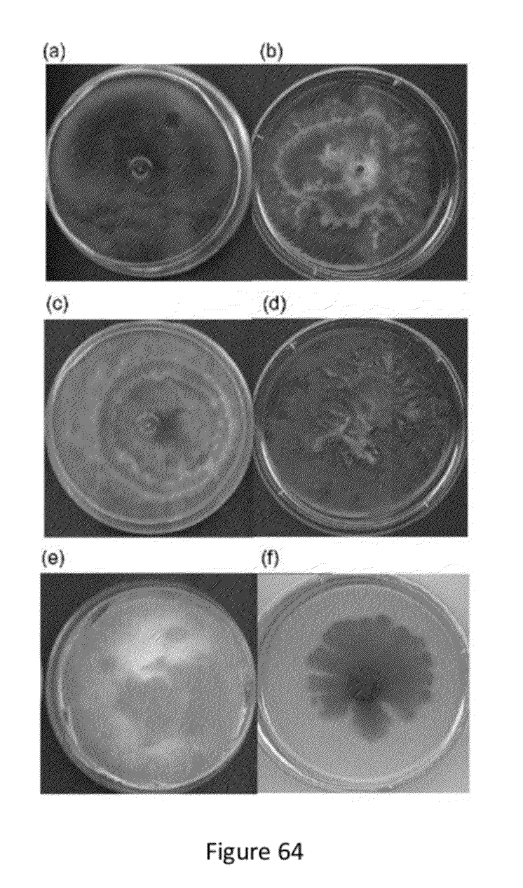

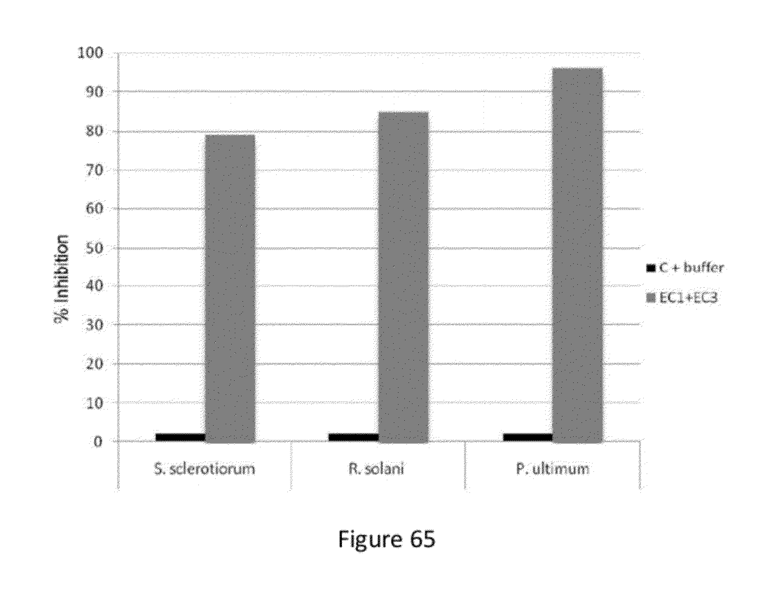

The mycostatic and mycotoxic effects of purified extracellular proteins EC1 (79 KDa) and EC3 (36 KDa) recovered from S. mycoparasitica cultures were assessed with three additional wide-spread plant pathogens that cause significant damage to agricultural crops. The inhibitory effects of a mixture of the EC1+EC3 extracellular S. mycoparasitica proteins (1:1) on S. sclerotiorum, R. solani, and P. ultimum, were assessed using the microtitre plate assay method disclosed by Ghosh (2006) and Yadav et al. (2007). Mycelia were collected and biomass weighed after 36 h of growth following the method taught by Strom et al. (2005, Co-cultivation of antifungal Lactobacillus plantarum MiLAB 393 and Aspergillus nidulans, evaluation of effects on fungal growth and protein expression. FEMS Microb Let, 246: 119-124). FIGS. 64(a), 64(c), and 64(e) are micrographs of control cultures of S. sclerotiorum, R. solani, and P. ultimum, respectively, grown on PDA for 4 days in the dark at 26° C. FIGS. 64(b), 64(d), and 64(f) are micrographs of S. sclerotiorum, R. solani, and P. ultimum, respectively, grown on PDA amended with a (1:1) mixture of the EC1 (79 kDa) and EC3 (36 kDa) exocellular proteins produced by S. mycoparasitica. The exocellular protein mixture caused significant mycelial damage to all three plant pathogen fungal cultures (FIGS. 64(b), 64(d), 64(f)). The data in FIG. 65 demonstrate that the exocellular protein mixture caused: (i) about 80% inhibition in growth of S. sclerotiorum compared to the untreated control, (ii) about 85% inhibition in the growth of R. solani compared to the untreated control, and (iii) about 95% inhibition in the growth of P. ultimum compared to the untreated control. These data demonstrate that the S. mycoparasitica extracellular proteins disclosed herein have mycostatic and mycotoxic effects on a broad range of soil and plant-pathogenic fungi.

The studies disclosed herein clearly demonstrate that exocellular proteins derived from S. mycoparasitica, identified as a 79 kDa protein comprising SEQ ID NO:36, a 50 kDa protein comprising SEQ ID NO:37, a 36 kDa protein comprising SEQ ID NO:38, and a 13 kDa protein comprising SEQ ID NO:39, are useful for inhibiting spore germination of various plant pathogenic fungi such as Fusarium spp., Sclerotinia spp., Rhizoctonia spp., and Pythium spp. The studies disclosed herein further demonstrate that contacting mycelia of plant pathogenic fungi with one or more of these exocellular proteins causes cessation of mycelial growth and results in lysis. Accordingly, transforming a plant cell with one or more nucleic acid molecules that encode one or more polypeptides comprising an amino acid sequences that shares at least 80% sequence identity with SEQ ID NO:36, SEQ ID NO:37, SEQ ID NO:38, or SEQ ID NO:39 will enable the plant cell to produce one or more polypeptide molecules comprising the amino acid sequences having at least 80% sequence identity with the amino acid sequence disclosed in SEQ ID NO:36, SEQ ID NO:37, SEQ ID NO:38, or SEQ ID NO:39. Accordingly, when such transformed cells are cultured into plants, the plants comprising the cells will express the polypeptides resulting in formation of the exocellular proteins within the cells. During the processes of colonizing the surfaces of such plants and/or infecting the plants, plant pathogenic fungi coming into proximity with the exocellular proteins and/or into contact with the exocellular proteins will be inhibited and or lysed.

While the present disclosure has been described with reference to what are presently considered to be the preferred examples, it is to be understood that the application is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

1. An isolated culture of Sphaerodes mycoparasitica, wherein the species is characterized by a combination of:
(a) ascospore size, shape (fusiform and triangular) and wall ornamentation (reticulate and smooth);
(b) conidia produced from simple phialides on the surface of ascoma peridial wall on ascoma surrounding hyphae, and on irregularly branched conidiophores arising from hyphae; and
(c) forming hook-like structures parasitizing living hyphae of Fusarium.

2. The isolated culture of Sphaerodes mycoparasitica according to claim 1, comprising a gene encoding a large subunit of ribosomal RNA gene as shown in SEQ ID NO:1 or a variant thereof.

3. The isolated culture of Sphaerodes mycoparasitica according to claim 1, comprising a gene encoding a small subunit of ribosomal RNA as shown in SEQ ID NO:2 or a variant thereof.

4. The isolated culture of Sphaerodes mycoparasitica according to claim 1, comprising a gene encoding an internal transcribed spacer ribosomal DNA as shown in SEQ ID NO:3 or a variant thereof.

5. The isolated culture of claim 1, wherein the isolated culture is Sphaerodes mycoparasitica strain IDAC 301008-01, IDAC 301008-02 or IDAC 301008-03.

6. A method of controlling pathogenic fungi comprising administering the culture of Sphaerodes mycoparasitica according to claim 1 to a subject or composition in need thereof.

7. The method of claim 6, wherein the pathogenic fungus is Fusarium spp., Sclerotinia spp., Rhizoctonia spp. or Pythium spp.

8. The method of claim 6, for controlling pathogenic fungi in plants.

9. The method of claim 6, for controlling pathogenic fungi in animals.

10. A method of modulating synthesis of one of a Fusarium trichothecene mycotoxin deoxynivalenol (DON), mycotoxin 3-ADON, mycotoxin 15-ADON, mycotoxin zerelanone, and mycotoxin aurofusarin, comprising administering the culture of Sphaerodes mycoparasitica according to claim 1 to a subject or composition in need thereof.

11. A composition comprising the culture of Sphaerodes mycoparasitica according to claim 1 and a carrier.

12. The composition of claim 11, further comprising an additional antifungal agent.

13. A method of modulating synthesis of one of a Fusarium trichothecene mycotoxin deoxynivalenol (DON), mycotoxin 3-ADON, mycotoxin 15-ADON, mycotoxin zerelanone, and mycotoxin aurofusarin comprising administering the composition according to claim 11 to a subject or composition in need thereof.

14. A method for controlling pathogenic fungi in plants, the method comprising treating a batch of seeds with the culture of claim 1 and then culturing the treated seeds into plants.

15. A method for controlling pathogenic fungi in plants, the method comprising treating a batch of seeds with the composition of claim 11 and then culturing the treated seeds into plants.

16. An isolated protein comprising the amino acid sequence as shown in SEQ ID NO:36, SEQ ID NO:37, SEQ ID NO:38 or SEQ ID NO:39 or a variant thereof.

17. The isolated protein of claim 16, wherein the protein is an exocellular protein recoverable from a culture of Sphaerodes mycoparasitica strain IDAC 301008-01, -02, or -03, said exocellular protein comprising the amino acid sequence as shown in SEQ ID NO:39 having a molecular weight of 13 kDa, said exocellular protein comprising the amino acid sequence as shown in SEQ ID NO:38 having a molecular weight of 36 kDa, said exocellular protein comprising the amino acid sequence as shown in SEQ ID NO:37 having a molecular weight of 50 kDa or said exocellular protein comprising the amino acid sequence as shown in SEQ ID NO:36 having the molecular weight of 79 kDa.

18. A method of controlling pathogenic fungi comprising administering the protein of claim 16 to a subject or composition in need thereof.

19. The method of claim 18, for controlling pathogenic fungi in plants.

20. The method of claim 18, for controlling pathogenic fungi in animals.

21. The method of claim 18, wherein the pathogenic fungus is one of a Fusarium spp., a Sclerotinia spp., a Rhizoctonia spp., or a Pythium spp.

22. A composition comprising the isolated protein of claim 16, and a carrier.

23. The composition according to claim 22, further comprising an additional antifungal agent.

24. A method for testing a sample of plant seeds for the presence therein of aurofusarin, the method comprising:
processing a portion of the sample of plant seeds to produce a DNA sample therefrom; and
processing the DNA sample with a PCR primer set comprising SEQ ID NO: 32 and SEQ ID NO: 33 to detect the presence and/or expression therein of a gene or nucleic acid sequence coding for aurofusarin.

25. An isolated nucleic acid molecule comprising a nucleotide sequence set forth in SEQ ID NO: 32 or SEQ ID NO: 33.

26. A method for detoxifying food, feed, or an environmental sample comprising one or more of a Fusarium trichothecene mycotoxin deoxynivalenol (DON), mycotoxin 3-ADON, mycotoxin 15-ADON, mycotoxin zerelanone, and mycotoxin aurofusarin comprising administering the culture of Sphaerodes mycoparasitica according to claim 1 to said food, feed, or environmental sample.
